(19) United States
(12) Patent Application Publication
(10) Pub. No.: US 2024/0016781 A1
(43) Pub. Date: Jan. 18, 2024

(54) TREATMENT OF KRAS MUTANT CANCERS

(71) Applicant: MEI Pharma, Inc., San Diego, CA (US)

(72) Inventors: Sandra E. Wiley, San Diego, CA (US); Daniel P. Gold, San Diego, CA (US)

(73) Assignee: MEI Pharma, Inc., San Diego, CA (US)

(21) Appl. No.: 18/253,481

(22) PCT Filed: Nov. 19, 2021
(86) PCT No.: PCT/US2021/060151

§ 371 Date: May 18, 2023

Related U.S. Application Data
(60) Provisional application No. 63/173,361, filed on Apr. 10, 2021.
(60) Provisional application No. 63/158,849, filed on Mar. 9, 2021.
(60) Provisional application No. 63/154,652, filed on Feb. 26, 2021.
(60) Provisional application No. 63/115,969, filed on Nov. 19, 2020.

(51) Int. Cl. A61K 31/4025; A61K 31/519; A61K 31/517; A61K 31/352; A61K 45/06; A61P 35/00

(52) U.S. Cl. CPC A61K 31/4025; A61K 31/519; A61K 31/517; A61K 31/352; A61K 45/06; A61P 35/00

(57) ABSTRACT

The disclosure herein provides combination therapies for the treatment of KRAS mutant cancers. The disclosure provides combination therapies of CDK inhibitors, e.g., a CDK inhibitor represented by Formula I or a pharmaceutically acceptable salt thereof together with an additional therapeutic agent, e.g., an anticancer agent.

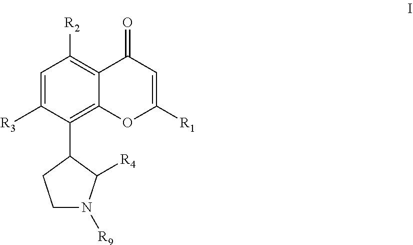

### CROSS-REFERENCE TO RELATED APPLICATIONS

This application is an International Application which claims priority to U.S. Provisional Application Nos. 63/115,969, filed Nov. 19, 2020, 63/154,652, filed Feb. 26, 2021, 63/158,849, filed Mar. 9, 2021, and 63/173,361, filed Apr. 10, 2021, each of which is incorporated herein by reference in its entirety.

### FIELD

The disclosure provides novel combination therapies including the CDK inhibitor voruciclib.

### BACKGROUND

Numerous cancer-related therapeutics are under phase I or phase II clinical trial and evaluations at any particular time; however, most of them will fail to advance. In fact, it is estimated that more than 90% of cancer-related therapeutics will fail phase I or II clinical trial evaluation. The failure rate in phase III trials is almost 50%, and the cost of new drug development from discovery through phase III trials is between $0.8 billion and $1.7 billion and can take between eight and ten years.

In addition, many patients fail to respond even to standard drugs that have been shown to be efficacious. For reasons that are not currently well understood or easily evaluated, individual patients may not respond to standard drug therapy. In some cases, administration of drug combinations may be more efficacious for treating cancer than drugs administered individually. These drug combinations may act synergistically to enhance the anti-cancer activity of the drugs. In some cases, drugs that are not particularly efficacious may find new and unexpected uses when combined with additional drug therapies.

### SUMMARY

In one aspect, the disclosure provides a method of treating a KRAS mutant cancer comprising administering to a subject in need thereof a therapeutically effective amount of a CDK inhibitor represented by Formula I.

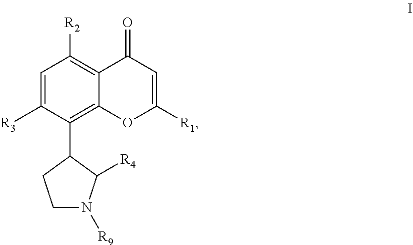

or a pharmaceutically acceptable salt thereof, wherein:
R₁ is optionally substituted phenyl;
R₂ and R₃ are each independently selected from hydroxy and OR₈, wherein R₈ is optionally substituted C₁-C₁₀-alkyl;
R₄ is optionally substituted C₁-C₄-alkyl; and
R₉ is hydrogen or optionally substituted C₁-C₄-alkyl.

In certain aspects, the disclosure provides a method of treating a KRAS mutant cancer comprising administering to a subject in need thereof a therapeutically effective amount of a CDK inhibitor represented by Formula I:

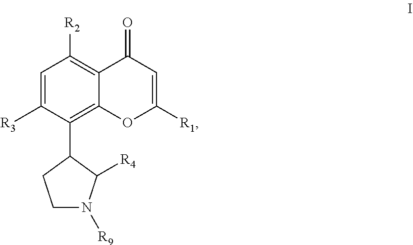

or a pharmaceutically acceptable salt thereof, wherein:
R₁ is optionally substituted phenyl;
R₂ and R₃ are each independently selected from hydroxy and OR₈, wherein R₈ is optionally substituted C₁-C₁₀-alkyl;
R₄ is optionally substituted C₁-C₄-alkyl; and
R₉ is hydrogen or optionally substituted C₁-C₄-alkyl;
   and a therapeutically effective amount of an additional therapeutic agent. In certain embodiments, the cancer is a blood cancer.

In certain embodiments, the compound of Formula I is represented by Formula Ia:

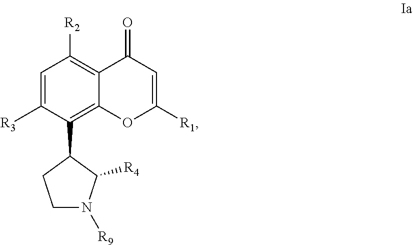

or a pharmaceutically acceptable salt thereof.

In certain embodiments for a compound or salt of Formula I or Ia, R₁ is optionally substituted with one or more substituents independently selected from hydroxy, cyano, halo, amino, C₁-C₄-alkyl, C₁-C₄-alkoxy, C₁-C₄-hydroxyalkyl, C₁-C₄-haloalkyl, and nitro. In certain embodiments, R₁ is substituted with one or more substituents independently selected from halo and C₁-C₄-haloalkyl. In certain embodiments, R₁ is 2-chloro-4-trifluoromethylphenyl.

In certain embodiments for a compound or salt of Formula I or Ia, R₂ and R₃ are each independently selected from hydroxy and OR₈, wherein R₈ is C₁-C₁₀-alkyl optionally substituted with one or more substituents independently selected from hydroxy, cyano, halo, amino, ═O, ═S, C₁-C₄-alkoxy, and nitro. In certain embodiments, R₂ and R₃ are each hydroxy.

In certain embodiments for a compound or salt of Formula I or Ia, R₄ is C₁-C₄-alkyl substituted with one or more substituents selected from hydroxy, cyano, halo, amino, ═O, ═S, C₁-C₄-alkoxy, and nitro. In certain embodiments, R₄ is C₁-C₄-alkyl substituted with one or more substituents selected from hydroxy, cyano, halo, amino, ═O, ═S, C₁-C₄-alkoxy, and nitro.

In certain embodiments, R₄ is 2-hydroxymethyl.

In certain embodiments for a compound or salt of Formula I or Ia, R₉ is C₁-C₄-alkyl optionally substituted with hydroxy, cyano, halo, amino, ═O, ═S, C₁-C₄-alkoxy, and nitro. In certain embodiments, R₉ is methyl. In certain embodiments the compound of Formula I is represented by formula Ib:

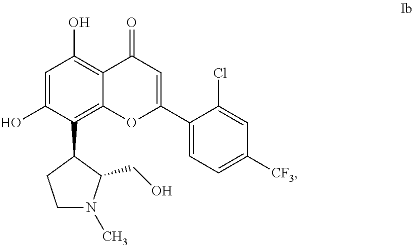 [SMILES: CN1CC[C@@H](c2c(O)cc(O)c3c(=O)cc(-c4ccc(C(F)(F)F)cc4Cl)oc23)[C@@H]1CO]

or a pharmaceutically acceptable salt thereof.

In certain embodiments, the blood cancer of the methods described herein is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphomas, extranodal marginal zone B-cell lymphomas, nodal marginal zone B-cell lymphoma, splenic marginal zone B-cell lymphoma, Burkitt lymphoma, lymphoplasmacytic lymphoma, and primary central nervous system lymphoma. The blood cancer may be diffuse large B-cell lymphoma, acute myeloid leukemia or chronic lymphocytic leukemia.

For certain methods described herein, the CDK inhibitor and additional therapeutic agent may be administered concurrently. For the methods described herein, the CDK inhibitor and additional therapeutic agent may be administered sequentially within about 12 hours of each other, such as within about 5 hours of each other.

For certain methods described herein, the CDK inhibitor and additional therapeutic agent may be co-formulated in a pharmaceutical composition.

For certain methods described herein, the CDK inhibitor and additional therapeutic agent may be administered daily, every other day or every third day.

In certain aspects the disclosure provides a pharmaceutical composition comprising a therapeutically effective amount of a CDK inhibitor represented by Formula I:

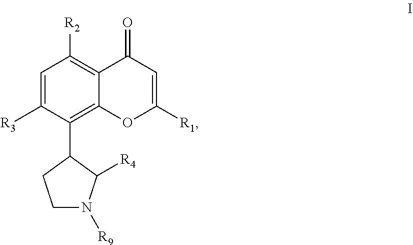

or a pharmaceutically acceptable salt thereof, wherein:
R₁ is optionally substituted phenyl;
R₂ and R₃ are each independently selected from hydroxy and OR₈, wherein R₈ is optionally substituted C₁-C₁₀-alkyl;
R₄ is optionally substituted C₁-C₄-alkyl; and
R₉ is hydrogen or optionally substituted C₁-C₄-alkyl;
   a therapeutically effective amount of an additional therapeutic agent, and a pharmaceutically acceptable excipient.

In certain embodiments, the compound or salt of Formula I is represented by Formula Ia:

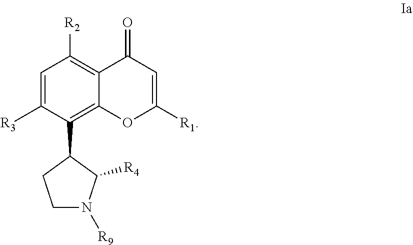

For the compositions described herein, for a compound or salt of Formula I or Ia, R₁ may be optionally substituted with one or more substituents independently selected from hydroxy, cyano, halo, amino, C₁-C₄-alkyl, C₁-C₄-alkoxy, C₁-C₄-hydroxyalkyl, C₁-C₄-haloalkyl, and nitro. In certain embodiments, R₁ is substituted with one or more substituents independently selected from halo and C₁-C₄-haloalkyl. In certain embodiments, R₁ is 2-chloro-4-trifluoromethylphenyl.

For the compositions described herein, for a compound or salt of Formula I or Ia, R₂ and R₃ may each independently selected from hydroxy and OR₈, wherein R₈ is C₁-C₁₀-alkyl optionally substituted with one or more substituents independently selected from hydroxy, cyano, halo, amino, ═O, ═S, C₁-C₄-alkoxy, and nitro. In certain embodiments, R₂ and R₃ are each hydroxy.

For the compositions described herein, for a compound or salt of Formula I or Ia, R₄ is C₁-C₄-alkyl substituted with one or more substituents selected from hydroxy, cyano, halo, amino, ═O, ═S, C₁-C₄-alkoxy, and nitro. In certain embodiments, R₄ is C₁-C₄-alkyl substituted with one or more substituents selected from hydroxy, cyano, halo, amino, ═O, ═S, C₁-C₄-alkoxy, and nitro. In certain embodiments, R₄ is 2-hydroxymethyl.

For the compositions described herein, for a compound or salt of Formula I or Ia, R₉ may be C₁-C₄-alkyl optionally substituted with hydroxy, cyano, halo, amino, ═O, ═S, C₁-C₄-alkoxy, and nitro. In certain embodiments, R₉ is methyl.

For the compositions described herein, the compound of Formula I may be represented by Formula Ib:

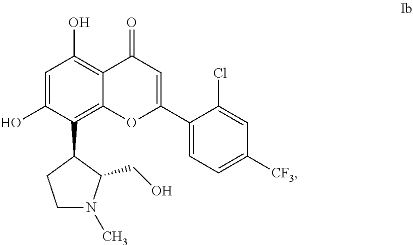 [SMILES: CN1CC[C@@H](c2c(O)cc(O)c3c(=O)cc(-c4ccc(C(F)(F)F)cc4Cl)oc23)[C@@H]1CO]

or a pharmaceutically acceptable salt thereof.

For the compositions described herein, the compound of Formula I may be (+)-trans-2-(2-chloro-4-trifluoromethylphenyl)-5,7-dihydroxy-8-(2-hydroxymethyl-1-methyl-pyrrolidin-3-yl)-chromen-4-one hydrochloride.

### BRIEF DESCRIPTION OF THE DRAWINGS

The foregoing summary, as well as the following detailed description of the disclosure, will be better understood when read in conjunction with the appended drawings.

FIG. 1 illustrates cell line responses to MEI-522 (Voruciclib) at 72 hours ranked by Response Area. Single agent activity of MEI-522 (Voruciclib) was extracted from the combination dose matrices. Cell line responses are ranked by median response.

FIGS. 2A-2B illustrate single agent responses stratified by KRAS status at 72 hours. KRAS status ranked by median Response Area for MEI-522 (Voruciclib). Single agent activity of MEI-522 (Voruciclib) was extracted from the growth inhibition combination dose matrices.

FIG. 3 illustrates a heatmap of combination activity of MEI-522 (Voruciclib) with various enhancers in the cell line panel after 72 hours. Combination activity is represented by Synergy Score as determined by the Chalice Analyzer. Chalice Analyzer provides a heatmap in matrix view comparing strength of the Synergy scores between the cell lines. Cell lines are ranked by synergy score of MEI-522 (Voruciclib) in combination with AMG510. Synergy scores (>7.71) are represented as red. Moderate synergy scores (between 3.92 and 7.71) are represented in shades of red. Low to moderate synergy scores (<3.92) are represented in white. Where a combination has not been assessed, this is represented by a grey box.

FIG. 4 illustrates dose response curves representations of cytostatic vs cytotoxic compound activity.

FIG. 5A illustrates a dose matrix; FIG. 5B illustrates the Loewe Model; FIG. 5C illustrates Loewe Excess.

FIG. 6 illustrates an injection array; drug mass delivered: Voruciclib: 5 μg; AMG-510: 15 μg; Adagrasib: 15 μg. Replicate Tumors: n≥5. Time Point: 24 hr. Cell Line; MIA PaCa-2. Biomarker: CC3, H&E.

FIG. 7 illustrates that Combining Voruciclib and Adagrasib results in enhanced tumor cell death. FIG. 7A illustrates Cell death around each microinjection site measured by nuclear condensation and fragmentation. FIG. 7B illustrates % Cells with pyknotic/fragmented nuclei (dead cells). Data represents 5 tumors 2 Adagrasib injection sites and 1 Veh, Voru and Combo injection sites per tumor; 4 sections imaged per tumor.

FIG. 8 illustrates that Combining Voruciclib and Adagrasib increases apoptotic cells. FIG. 8A apoptosis measured by CC3+ cells; apoptosis in combination site is increasing in radial zones where single agent apoptosis is falling. FIG. 8B illustrates % of Cell Area CC3+. Data represents 5 tumors 2 Adagrasib injection sites and 1 Veh, Voru and Combo injection sites per tumor; 4 sections imaged per tumor. Error Bars are Std Err Mean.

FIG. 9 illustrates that Combining Voruciclib and AMG-510 results in enhanced tumor cell death. FIG. 9A illustrates Cell death around each microinjection site measured by nuclear condensation and fragmentation. FIG. 9B illustrates % Cells with pyknotic/fragmented nuclei (dead cells). Data represents 5 tumors with duplicate combination and AMG-510 injection sites per tumor, single injection sites other conditions; 4 sections imaged per tumor.

FIG. 10 illustrates that Combining Voruciclib and AMG-510 increases apoptotic cells. FIG. 10A illustrates apoptosis measured by CC3+ cells; combination induced apoptosis meets threshold for synergy, not just additivity. FIG. 10B illustrates % of Cell area CC3+. Data represents 5 tumors with duplicate combination and AMG-510 injection sites per tumor, single injection sites other conditions; 4 sections imaged per tumor. Error Bars=Stnd Err Mean.

FIGS. 11A-11B depict data in HCC-44 NSCLC cell tumors demonstrating that the combination of voruciclib and adagrasib increases apoptosis.

FIGS. 12A-12B depict data in HCC-44 xenografts demonstrating that the combination of voruciclib and AMG-510 increases cell death in some tumors.

FIGS. 13A-13B depict morphological data in HCC-44 xenografts.

FIGS. 14A-14B depict data in MIA PaCa-2 xenografts demonstrating that the combination of voruciclib and onvansertib (a PLK1 inhibitor) increases apoptotic cells in a pancreatic cancer model.

FIGS. 15A-15B depict data in Huh-7 xenografts demonstrating that the combination of voruciclib and onvansertib as well as the single agents do not result in an increase in apoptotic cells in a liver cancer model.

FIGS. 16A-16B depict data in an H441 lung cancer model ((NSCLC) KRAS G12V) demonstrating that combining voruciclib and onvansertib increases apoptotic cells.

FIGS. 17A-17B depict data in an H441 lung cancer model demonstrating that combining voruciclib and onvansertib results in enhanced tumor cell death.

FIGS. 18A-18B demonstrate that the combination of voruciclib and ME-344 in MIA PaCa-2 xenografts results in faster cell death than voruciclib alone, leading to larger area of post-apoptotic a-cellularity.

FIGS. 19A-19B demonstrate that combining voruciclib and ME-344 in MIA PaCa-2 xenografts results in enhanced tumor cell death (note that the morphological phenotype was easier to observe above background).

FIG. 20 depicts results of the combination of voruciclib with adagrasib PaCa-2 KRAS G12C mutant PDAC tumors, wherein the combination was found to decrease MYC positive cells more than either single agent.

FIG. 21 depicts cMYC and MCL-1 staining in MIA PaCa-2 PDAC tumors.

FIGS. 22A-22B provide data demonstrating that the combination injection of voruciclib and adagrasib in MIA Paca-2 PDAC tumors decreases MYC positive cells more than either single agent.

FIGS. 23A-23B show that the combination of voruciclib and onvansertib result in enhanced tumor cell death in HCC-44 (NSCLC) cells.

FIGS. 24A-24B show that the combination of voruciclib and onvansertib slightly increases apoptotic cells. The study was performed in HCC-44 (NSCLC) cells and apoptosis was measured by CC3+ cells.

FIG. 25 depicts the 3D spheroids for sotorasib and voruciclib in NCI-H358 cells after 1 day of exposure.

FIGS. 26A-26B depict the 3D spheroids for the voruciclib combination screening in NCI-H358 cells.

FIGS. 27A-27B depict the 3D spheroids for the sotorasib combination screening in NCI-H358 cells (NSCLC). FIG. 27A provides pictures of the NCI-H358 cells after 3 days of exposure with sotorasib, before ATP-Lite measurement. FIG. 27B is a dose response curve of sotorasib in NCI-H358 cells.

FIG. 28 depicts the 3D spheroids for the voruciclib+sotorasib combination screening in NCI-H358 cells (NSCLC). Pictures of the NCI-H358 cells were obtained after 3 days of exposure with the sotorasib and voruciclib combination.

FIGS. 29A-29B depict the synergy with voruciclib+sotorasib (at higher voruciclib concentrations) in NCI-H358 3D spheroids. FIG. 29A is a chart of the excess over Bliss score. FIG. 29B is a chart of the viability for well (% untreated).

FIGS. 30A-30B—CDK9 regulates transcription of MYC by RNA Pol II and MYC protein stability: Schematic illustrating (FIG. 30A) P-TEFb regulation of RNA Pol II driven transcription of MYC and (FIG. 30B) KRAS-ERK1 signaling pathway and regulation of MYC protein stability by phosphorylation of Ser 62. Proteins with decreased phosphorylation after voruciclib treatment are circled in purple. Points of CDK9 inhibition by voruciclib are noted.

FIGS. 31A-31D—Voruciclib induces rapid down regulation of RNA Pol II associated proteins that control transcription of MYC: Landscape of the voruciclib-sensitive phosphoproteome in MIA Paca-2 cells reveals rapid downregulation of phosphoproteins controlling transcription of MYC. Cells were treated with voruciclib (4 μM) over time. (FIG. 31A) Summary of peptide quantification after TMT labelling and analysis by LC-MS/MS. (FIG. 31B) Volcano plots of phosphosites (log 2 fold change vs −log 10 p-value). Significantly downregulated are in red. Significantly upregulated are in green (p<0.05. Fold change >2.0). (FIG. 31C) Summary of significantly down-regulated phosphoproteins and phosphopeptides over time. (FIG. 31D) Downregulated phosphoproteins with a role in regulation of RNA Pol II activity.

FIGS. 32A-32B—Voruciclib causes rapid inhibition MYC pSer62 phosphorylation and reduces MYC protein levels. Immunoblot analyses of c-MYC, phospho-c-MYC (Ser62), and actin in MIA Paca-2 KRAS G12C mutant PDAC cells. (FIG. 32A) Cells were treated with vehicle control, voruciclib (VOR, 4 μM), or the AZD4573 (AZD, a CDK9 inhibitor, 400 nM) for the indicated times. (FIG. 32B) Cells were treated with various concentrations of voruciclib or AZD4573 for the indicated time. Relative densitometry values are indicated.

FIGS. 33A-33D—Voruciclib inhibits proliferation of KRAS mutant cancer cell lines in vitro and in vivo: (FIG. 33A) Voruciclib IC50 values across multiple cell lines with KRAS mutations. Murine xenograft experiment showing tumor growth over time in mice bearing HCT-116 (G13D) (FIG. 33B), SW-480 (G12V) (FIG. 33C) or H-460 (Q61H) (FIG. 33D) tumors after treatment with voruciclib (VOR) at various doses (p.o.) for 11-14 days.

FIG. 34—Voruciclib synergizes with KRAS G12C inhibitors in vitro: Heatmap of combination activity of voruciclib with KRAS G12C inhibitors in cancer cell lines after 72 hours. Cell lines are ranked by synergy score of voruciclib in combination with either AMG510 or MTRX849. HSA, Bliss, and Loewe analyses were performed to generate the synergy scores using Chalice Analyzer. High synergy scores are represented as dark green. Moderate synergy scores are represented in shades of green. Low to moderate synergy scores are represented in white. Cell sensitivity to KRAS G12C inhibitors are ranked by EC50 scores. High (<0.1 μM), Moderate (>0.1 μM), low (>1 μM). Where sensitivities to the two inhibitors differ, a range of responses is given.

FIGS. 35A and 35B illustrate decreased c-MYC Expression in Solid Tumors: 10 gene biomarkers evaluated in Phase 1 daily dosing study; c-MYC expression decreased in 17/25 patients (68%).

FIGS. 36A-36C illustrate CR in a Patient with Pulmonary Metastases; FIG. 36A: baseline CT scan; FIG. 36B: 2 months after starting the trial, radiological CR based on official radiological report; FIG. 36C: 14 months after starting the trail, patient remained on trial for 12 months only, and CR remained durable for 14 months.

FIGS. 37A-37D illustrate that Voruciclib Shows Preferential Tumor Accumulation in a Preclinical Model.

FIGS. 38A and 38B illustrate that Voruciclib Synergizes with Venetoclax in Venetoclax Sensitive and Resistant Cell Lines.

### DETAILED DESCRIPTION

While preferred embodiments are shown and described herein, such embodiments are provided by way of example only and are not intended to otherwise limit the scope of the disclosure, and various alternatives to the described embodiments of the disclosure may be employed.

### Definitions

Unless defined otherwise, all technical and scientific terms used herein have the same meaning as is commonly understood by one of skill in the art to which this disclosure belongs. All patents and publications referred to herein are incorporated by reference in their entireties.

The disclosure provides combination therapies for the treatment of cancer. In particular, the disclosure provides combination therapies of CDK inhibitors with other anticancer agents for treating cancer. In one aspect the disclosure provides compositions and methods for treating KRAS mutant cancers with a CDK inhibitor in combination with an additional therapeutic agent. Such combination provides synergistic effects in the treatment of cancers and particularly treatment of blood cancers, e.g., leukemia and lymphoma.

The general terms used hereinbefore and hereinafter preferably have the following meanings within the context of this disclosure, unless otherwise indicated. Thus, the definitions of the general terms as used in the context of the present disclosure are provided herein below.

The singular forms “a,” “an,” and “the” include plural reference unless the context clearly dictates otherwise.

The term “about,” as used herein, generally refers to an acceptable error range for the particular value as determined by one of ordinary skill in the art, which may depend in part on how the value is measured or determined. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, and within 2-fold, of a value.

As used herein, the term “at least one” is refers to one or more. For instance, the term “at least one anticancer agent” means that the combination comprises a single anticancer agent or more anticancer agents.

The term “effective amount” or “therapeutically effective amount,” as used herein, generally refers to an amount of a compound described herein that is sufficient to affect an intended, predetermined or prescribed application, including but not limited to, disease or condition treatment. The therapeutically effective amount can vary depending upon the application (e.g., in vitro or in vivo), or the subject and disease condition being treated, e.g., the weight and age of the subject, the severity of the disease condition and the manner of administration. The term also may apply to a dose that induces a particular response in target cells, e.g., reduction of proliferation or down regulation of activity of a target protein. The specific dose may vary depending on the particular compounds chosen, the dosing regimen to be followed, whether it is administered in combination with other compounds, timing of administration, the tissue to which it is administered, and the physical delivery system in which it is carried.

As used herein, the term “free base dosage” refers to an amount of drug in its free base form, which can be replaced by an amount of a salt of the drug by using a salt conversion factor.

As used herein, the term “pharmaceutically acceptable” means that the carrier, diluent, excipients, and/or salt must be compatible with the other ingredients of the formulation, and not deleterious to the recipient thereof “Pharmaceutically acceptable” also means that the compositions or dosage forms are within the scope of sound medical judgment, suitable for use for an animal or human without excessive toxicity, irritation, allergic response, or other problem or complication, commensurate with a reasonable benefit/risk ratio.

As used herein, the term “combination” or “pharmaceutical combination” refers to the combined administration of the anticancer agents. Combinations of the disclosure include a CDK inhibitor, e.g., a compound of Formula I, Ia, or Ib, and at least an additional therapeutic agent, e.g., an anti-cancer agent, which anti-cancer agents may be administered to a subject in need thereof, e.g., concurrently or sequentially. The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

A “therapeutic effect” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “solid form” may refer to a crystalline solid form or phase, including a crystalline free base and a crystalline salt.

The terms “co-administration,” “co-administering,” “administered in combination with,” and “administering in combination with” as used herein, encompass administration of two or more agents to a subject so that both agents and/or their metabolites are present in the subject at the same time. Co-administration includes simultaneous administration in separate compositions, administration at different times in separate compositions, or administration in a composition in which two or more agents are present.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells (e.g., CDK inhibition). The specific dose will vary depending on the particular compounds chosen, the dosing regimen to be followed, whether the compound is administered in combination with other compounds, timing of administration, the tissue to which it is administered, and the physical delivery system in which the compound is carried.

The term “pharmaceutically acceptable salt” refers to salts derived from a variety of organic and inorganic counter ions, including fumarate, maleate, phosphate, L-tartrate, esylate, besylate, hydrobromide, hydrochloride, citrate, gentisate, oxalate, sulfate counter ions, and the like. Pharmaceutically acceptable acid addition salts can be formed with inorganic acids and organic acids.

“Pharmaceutically acceptable carrier” or “pharmaceutically acceptable excipient” is intended to include any and all solvents, dispersion media, coatings, antibacterial and antifungal agents, isotonic and absorption delaying agents. Except insofar as any conventional media or agent is incompatible with the active ingredient, its use in the therapeutic compositions of the disclosure is contemplated. Supplementary active ingredients can also be incorporated into the described compositions.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “extragranular” refers to substances that are outside of a granule, e.g., a substance added to granules (multiparticle compacts formed by a granulation process) and physically mixed with granules, but not contained within the granules.

The term “intragranular” refers to substances that are within a granule (a multiparticle compact formed by a granulation process). Granules may be formed by processes such as wet granulation (i.e., prepared using moisture or steam, thermal, melt, freeze, foam, and other processes) or dry granulation.

The term “acidulant” refers to a substance that increases acidity.

The terms “transmission” or “transmission mode,” when used in conjunction with powder X-ray diffraction, refers to the transmission (also known as Debye-Scherrer) sampling mode. The terms “reflection” or “reflection mode,” when used in conjunction with powder X-ray diffraction, refers to the reflection (also known as Bragg-Brentano) sampling mode.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by 13C- or 14C-enriched carbons, are within the scope of this disclosure.

When ranges are used herein to describe, for example, physical or chemical properties such as molecular weight or chemical formulae, all combinations and subcombinations of ranges and specific embodiments therein are intended to be included. Use of the term “about” or “approximately” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

The term “synergistic,” or “synergistic effect” or “synergism” as used herein, generally refers to an effect such that the one or more effects of the combination of compositions is greater than the one or more effects of each component alone, or they can be greater than the sum of the one or more effects of each component alone. The synergistic effect can be greater than about 10%, 20%, 30%, 50%, 75%, 100%, 110%, 120%, 150%, 200%, 250%, 350%, or 500% or more than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. The combination index (CI) method of Chou and Talalay may be used to determine the synergy, additive or antagonism effect of the agents used in combination. When the CI value is less than 1, there is synergy between the compounds used in the combination; when the CI value is equal to 1, there is an additive effect between the compounds used in the combination and when CI value is more than 1, there is an antagonistic effect. The synergistic effect may be attained by co-formulating the agents of the pharmaceutical combination. The synergistic effect may be attained by administering two or more agents as separate formulations administered simultaneously or sequentially.

Cyclin-dependent kinases (CDKs) are a family of enzymes which become activated in specific phases of the cell cycle. CDKs consist of a catalytic subunit (the actual cyclin-dependent kinase or CDK) and a regulatory subunit (cyclin). There are at least nine CDKs (CDK1, CDK2, CDK3, CDK4, CDK5, CDK6, CDK7, CDK8, CDK9, etc.) and at least 15 different types of cyclins (cyclin A, B1, B2, D1, D2, D3, E, H etc.). Each step of the cell cycle is regulated by such CDK complexes: G1/S transition (CDK2/cyclin A, CDK4/cyclin D1-D3, CDK6/cyclin D3), S phase (CDK2/cyclin A), G2 phase 30 (CDK1/cyclin A), G2/M transition phase (CDK1/cyclin B).

As used herein, the term “CDK inhibitor” refers to an agent that is capable of inhibiting one or more cyclin dependent kinases (CDK). Aberrant expression and overexpression of these kinases are evidenced in many disease conditions such as cancer. In the context of the present disclosure, the CDK inhibitor of the pharmaceutical combination described herein may be a compound of Formula I, Ia, or Ib or a pharmaceutically acceptable salt thereof. The compounds of the present disclosure may inhibit one or more of CDK1/cyclin B, CDK2/cyclin E, CDK4/cyclin D, CDK4/cyclin D1 and CDK9/cyclin T1 with specificity. In certain embodiments, a compound of the disclosure inhibits CDK9/cyclin T1 or CDK9 with specificity.

Disclosed herein are combination therapies for the treatment of cancer, e.g., leukemia, lymphoma and breast cancer. The methods and compositions described herein may include a cyclin-dependent kinase (CDK) inhibitor, such as a compound of Formula I, Ia, or Ib or a pharmaceutically acceptable salt thereof.

In certain embodiments, a CDK inhibitor of the disclosure is represented by a compound disclosed in U.S. Pat. Nos. 7,271,193; 7,915,301; 8,304,449; 7,884,127; 8,563,596, the entire contents of each of which are incorporated herein by reference. In certain embodiments, a CDK inhibitor of the disclosure is represented by Formula I:

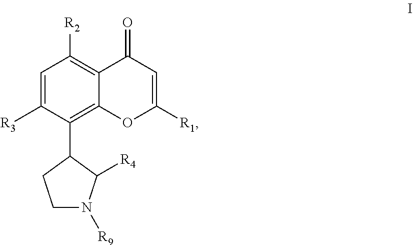

or a pharmaceutically acceptable salt thereof, wherein:
R₁ is optionally substituted phenyl;
R₂ and R₃ are each independently selected from hydroxy and OR₈, wherein R₈ is optionally substituted C₁-C₁₀-alkyl;
R₄ is optionally substituted C₁-C₄-alkyl; and
R₉ is hydrogen or optionally substituted C₁-C₄-alkyl.

In certain embodiments, the compound or salt of Formula I is represented by Formula Ia:

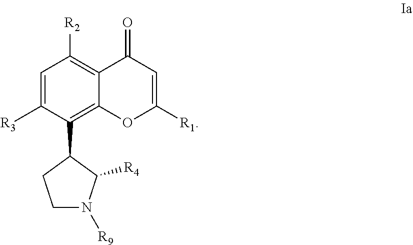

In certain embodiments for a compound or salt of Formula I or Ia, R₁ is optionally substituted with one or more substituents independently selected from hydroxy, cyano, halo, amino, C₁-C₄-alkyl, C₁-C₄-alkoxy, C₁-C₄-hydroxyalkyl, C₁-C₄-haloalkyl, and nitro. In certain embodiments, R₁ is substituted with one or more substituents independently selected from hydroxy, cyano, halo, C₁-C₄-alkyl, and C₁-C₄-haloalkyl. In certain embodiments, R₁ is substituted with one or more substituents independently selected from halo and C₁-C₄-haloalkyl. In certain embodiments, R₁ is 2-chloro-4-trifluoromethylphenyl.

The term “alkyl” refers to a straight or branched hydrocarbon chain radical consisting solely of carbon and hydrogen atoms, and containing no unsaturation. In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more substituents such as those substituents described herein.

The term “alkoxy” refers to a radical bonded through an oxygen atom of the formula —O-alkyl, where alkyl is an alkyl chain as defined above.

The term “amino” refers to the group —NR′R″, wherein R′ and R″ are independently selected from hydrogen; and alkyl, hydroxyl, aryl, cycloalkyl, heterocycloalkyl, and heteroaryl, any one of which may be optionally substituted with one or more substituents such as hydroxy, cyano, halo, amino, C₁-C₄-alkyl, C₁-C₄-alkoxy, C₁-C₄-hydroxyalkyl, C₁-C₄-haloalkyl, and nitro.

The term “C_{x-y}” when used in conjunction with a chemical moiety, such as alkyl is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The term “haloalkyl” refers to an alkyl group that is substituted by one or more halo radicals, for example, trifluoromethyl, difluoromethyl, fluoromethyl, 2,2,2-trifluoroethyl, 1-chloromethyl-2-fluoroethyl, and the like. In some embodiments, the alkyl part of the haloalkyl is further optionally substituted as described herein.

The term “hydroxyalkyl” refers to an alkyl group that is substituted by one or more hydroxy radicals, for example, hydroxymethyl, hydroxyethyl, dihydroxymethyl, and the like. In some embodiments, the alkyl part of the hydroxyalkyl is further optionally substituted as described herein.

In certain embodiments for a compound or salt of Formula I or Ia, R₂ and R₃ are each independently selected from hydroxy and OR₈, wherein R₈ is C₁-C₁₀-alkyl optionally substituted with one or more substituents independently selected from hydroxy, cyano, halo, amino, ═O, ═S, C₁-C₄-alkoxy, and nitro. In certain embodiments, R₈ at each occurrence is selected from optionally substituted C₁-C₆-alkyl, such as optionally substituted C₁-C₄-alkyl. In certain embodiments, R₂ and R₃ are each independently hydroxy.

In certain embodiments for a compound or salt of Formula I or Ia, R₄ is optionally substituted C₁-C₄-alkyl, wherein R₄ is optionally substituted with one or more substituents selected from hydroxy, cyano, halo, amino, ═O, ═S, C₁-C₄-alkoxy, and nitro. In certain embodiments, R₄ is optionally substituted C₁-C₂-alkyl. In certain embodiments, R₄ is hydroxyalkyl, e.g., 2-hydroxymethyl.

In certain embodiments for a compound or salt of Formula I or Ia, R₉ is C₁-C₄-alkyl optionally substituted with hydroxy, cyano, halo, amino, ═O, ═S, C₁-C₄-alkoxy, and nitro. In certain embodiments, R₉ is optionally substituted C₁-C₂-alkyl. In certain embodiments, R₉ is methyl. In certain embodiments, R₉ is hydrogen.

In certain embodiments for a compound or salt of Formula I or Ia, a compound of Formula I is a compound or pharmaceutically acceptable salt selected from: (+)-trans-2-(2-Chloro-4-trifluoromethylphenyl)-8-(2-hydroxymethyl-1-methyl-pyrrolidin-3-yl)-5,7-dimethoxy-chromen-4-one; (+)-trans-2-(2-Chloro-4-trifluoromethylphenyl)-5,7-dihydroxy-8-(2-hydroxymethyl-1-methylpyrrolidin-3-yl)-chromen-4-one; and (+)-trans-2-(2-Chloro-4-trifluoromethylphenyl)-5,7-dihydroxy-8-(2-hydroxymethyl-1-methyl-pyrrolidin-3-yl)-chromen-4-one hydrochloride.

In certain embodiments, the compound of Formula I or Ia is represented by Formula Ib:

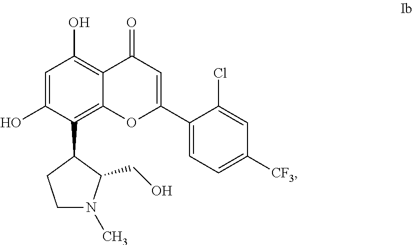 [SMILES: CN1CC[C@@H](c2c(O)cc(O)c3c(=O)cc(-c4ccc(C(F)(F)F)cc4Cl)oc23)[C@@H]1CO]

or a pharmaceutically acceptable salt thereof. In certain embodiments, the compound of Formula I, Ia, or Ib is in the form of an acid addition salt, such as the hydrochloride salt.

In certain embodiments, the CDK inhibitor of the disclosure is a polymorph of Formula Ib (i.e. voruciclib) disclosed in International Application No. WO 2020/210760 (PCT/US2020/027847), which is incorporated by reference herein.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, as well as represents a stable compound, which does not readily undergo transformation such as rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

Substituents can include any substituents described herein, for example, a halogen, a hydroxy, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic and heteroaromatic moiety. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

Procedures for the manufacture of the compounds of Formula I, Ia, and Ib or the pharmaceutically acceptable salts thereof, may be found in PCT Patent Publication No. WO2004004632 (corresponding to U.S. Pat. No. 7,271,193) and PCT Patent Publication No. WO2007148158.

The present disclosure provides pharmaceutically-acceptable salts of any compound described herein, e.g., a compound of Formula I, Ia, Ib. Pharmaceutically-acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some cases, a pharmaceutically-acceptable salt is a metal salt. In some cases, a pharmaceutically-acceptable salt is an ammonium salt.

Acid addition salts can arise from the addition of an acid to a compound described herein. In some cases, the acid is organic. In some cases, the acid is inorganic. Non-limiting examples of suitable acids include hydrochloric acid, hydrobromic acid, hydroiodic acid, nitric acid, nitrous acid, sulfuric acid, sulfurous acid, a phosphoric acid, nicotinic acid, isonicotinic acid, lactic acid, salicylic acid, 4-aminosalicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, citric acid, oxalic acid, maleic acid, hydroxymaleic acid, methylmaleic acid, glycolic acid, malic acid, malonic acid, cinnamic acid, mandelic acid, 2-phenoxybenzoic acid, 2-acetoxybenzoic acid, embonic acid, phenylacetic acid, N-cyclohexylsulfamic acid, methanesulfonic acid, ethanesulfonic acid, benzenesulfonic acid, p-toluenesulfonic acid, 2-hydroxyethanesulfonic acid, ethane-1,2-disulfonic acid, 4-methylbenzenesulfonic acid, naphthalene-2-sulfonic acid, naphthalene-1,5-disulfonic acid, 2-phosphoglyceric acid, 3-phosphoglyceric acid, glucose-6-phosphoric acid, and an amino acid.

Metal salts can arise from the addition of an inorganic base to a compound of the disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound described herein. Non-limiting examples of suitable organic amines include triethyl amine, diisopropyl amine, ethanol amine, diethanol amine, triethanol amine, morpholine, N-methylmorpholine, piperidine, N-methylpiperidine, N-ethylpiperidine, dibenzyl amine, piperazine, pyridine, pyrrazole, pipyrrazole, imidazole, pyrazine, pipyrazine, ethylenediamine, N,N′-dibenzylethylene diamine, procaine, chloroprocaine, choline, dicyclohexyl amine, and N-methylglucamine.

Non-limiting examples of suitable ammonium salts include is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzyl amine salt, a piperazine salt, a pyridine salt, a pyrrazole salt, a pipyrrazole salt, an imidazole salt, a pyrazine salt, a pipyrazine salt, an ethylene diamine salt, an N,N′-dibenzylethylene diamine salt, a procaine salt, a chloroprocaine salt, a choline salt, a dicyclohexyl amine salt, and a N-methylglucamine salt.

Non-limiting examples of suitable acid addition salts include a hydrochloride salt, a hydrobromide salt, a hydroiodide salt, a nitrate salt, a nitrite salt, a sulfate salt, a sulfite salt, a phosphate salt, a malonate salt, a hydrogen phosphate salt, a dihydrogen phosphate salt, a carbonate salt, a bicarbonate salt, a nicotinate salt, an isonicotinate salt, a lactate salt, a salicylate salt, a 4-aminosalicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a citrate salt, an oxalate salt, a maleate salt, a hydroxymaleate salt, a methylmaleate salt, a glycolate salt, a malate salt, a cinnamate salt, a mandelate salt, a 2-phenoxybenzoate salt, a 2-acetoxybenzoate salt, an embonate salt, a phenylacetate salt, an N-cyclohexylsulfamate salt, a methanesulfonate salt, an ethanesulfonate salt, a benzenesulfonate salt, a p-toluenesulfonate salt, a 2-hydroxyethanesulfonate salt, an ethane-1,2-disulfonate salt, a 4-methylbenzenesulfonate salt, a naphthalene-2-sulfonate salt, a naphthalene-1,5-disulfonate salt, a 2-phosphoglycerate salt, a 3-phosphoglycerate salt, a glucose-6-phosphate salt, and an amino acid salt.

The compounds described herein, e.g., the compounds and salts of Formulas I, Ia, Ib, may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The compounds described herein, e.g., the compounds and salts of Formulas I, Ia, Ib, include the use of amorphous forms as well as crystalline forms (also known as polymorphs). The compounds described herein may be in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

The compounds described herein, e.g., the compounds and salts of Formulas I, Ia, Ib, include compounds that exhibit their natural isotopic abundance, and compounds where one or more of the atoms are artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared.

Compounds described herein, e.g., the compounds and salts of Formulas I, Ia, Ib, wherein the compound has carbon-carbon double bonds or carbon-nitrogen double bonds may exist, where applicable, in Z- or E-form (or cis- or trans-form). Furthermore, some chemical entities may exist in various tautomeric forms. Unless otherwise specified, chemical entities described herein are intended to include all Z-, E- and tautomeric forms as well.

In certain cases, a compound described herein may be a prodrug, e.g., wherein a carboxylic acid present in the parent compound is presented as an ester. The term “prodrug” is intended to encompass compounds which, under physiologic conditions, are converted into pharmaceutical agents, i.e., parent compound, of the present disclosure. One method for making a prodrug is to include one or more selected moieties which are hydrolyzed under physiologic conditions to reveal the desired molecule. In certain embodiments, the prodrug is converted by an enzymatic activity of the host animal such as enzymatic activity in specific target cells in the host animal. For example, esters or carbonates (e.g., esters or carbonates of alcohols or carboxylic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. For example, the prodrug may have improved cell permeability over the parent compound. The prodrug may also have improved solubility in pharmaceutical formulations over the parent drug. In some embodiments, the design of a prodrug increases the lipophilicity of the pharmaceutical agent. In some embodiments, the design of a prodrug increases the effective water solubility.

In certain embodiments, a cyclin-dependent kinase (CDK) inhibitor, e.g., a compound or salt of Formula I, Ia or Ib, may be used in combination with an additional therapeutic agent, e.g., an anti-cancer agent. In some embodiments, the additional therapeutic agent may be a KRAS inhibitor, e.g., a KRAS G12C inhibitor.

### Crystalline Forms

In an embodiment, the disclosure provides a crystalline solid form of Formula Ib (i.e., voruciclib). In an embodiment, the disclosure provides a crystalline solid form of voruciclib free base. In an embodiment, the disclosure provides a crystalline solid form of a voruciclib salt. The disclosure provides polymorphs, for example crystal forms, of voruciclib. In some embodiments, the polymorphs include free base voruciclib. In some embodiments, the polymorphs include voruciclib salts including a counterion corresponding to an acid selected from 1,5-naphthalenedisulfonic acid, 1-hydroxy-2-naphthoic acid, benzenesulfonic acid, benzoic acid, dibenzoyl-L-tartaric acid, ethanesulfonic acid, gentisic acid, hydrobromic acid, hydrochloric acid, maleic acid, malonic acid, oxalic acid, ortho-phosphoric acid, sulfuric acid, p-toluenesulfonic acid, and the like.

Any crystalline form described herein can be characterized by X-ray diffraction. In some embodiments, X-ray diffraction refers to X-ray powder diffraction. In some embodiments, X-ray diffraction may be measured using transmission mode or reflection mode. In an embodiment, the X-ray diffraction pattern of any embodiments herein is measured in transmission mode. In an embodiment, the X-ray diffraction pattern of any embodiments herein is measured in reflection mode. It is known in the art that an X-ray powder diffraction pattern may be obtained which has one or more measurement errors depending on measurement conditions (such as equipment, sample preparation, or instrument used). In particular, it is generally known that intensities in an X-ray powder diffraction pattern may vary depending on measurement conditions and sample preparation. For example, persons skilled in the art of X-ray powder diffraction will realize that the relative intensities of peaks may vary according to the orientation of the sample under test and based on the type and settings of the instrument used. The skilled person will also realize that the position of reflections can be affected by the precise height at which the sample sits in the diffractometer, the sample's surface planarity, and the zero calibration of the diffractometer. Hence a person skilled in the art will appreciate that the diffraction pattern data presented herein is not to be construed as absolute and any crystalline form that provides a power diffraction pattern substantially the same as those disclosed herein fall within the scope of the present disclosure. For further information, see Jenkins and Snyder, Introduction to X-Ray Powder Diffractometry, John Wiley & Sons, 1996.

Different crystalline form may provide surprising advantages compared to non-crystalline forms, including improved thermodynamic stability, faster dissolution rate, improved performance in the stomach and gastric environment (including the avoidance of, or reduced, precipitation from solution upon a change to higher pH), improved exposure in mammals, and superior processability for formulation of drug into finished products suitable for patients.

In one embodiment, the disclosure provides a crystal form of voruciclib malonate, and/or a polymorph crystal form of voruciclib malonate (Mao1), characterized by an X-ray powder diffraction pattern including one or more peaks selected from:

| No | 2θ (°) | D (Å) | I (%) |
|---|---|---|---|
| 1 | 6.36 | 13.88 | 11 |
| 2 | 7.31 | 12.08 | 28 |
| 3 | 9.34 | 9.46 | 15 |
| 4 | 10.05 | 8.79 | 12 |
| 5 | 13.59 | 6.51 | 31 |
| 6 | 14.08 | 6.28 | 29 |
| 7 | 15.21 | 5.82 | 76 |
| 8 | 15.67 | 5.65 | 65 |
| 9 | 17.53 | 5.06 | 27 |
| 10 | 18.70 | 4.74 | 23 |
| 11 | 18.98 | 4.67 | 100 |
| 12 | 19.38 | 4.58 | 36 |
| 13 | 19.67 | 4.51 | 63 |
| 14 | 20.16 | 4.40 | 14 |
| 15 | 20.39 | 4.35 | 12 |
| 16 | 21.01 | 4.23 | 13 |
| 17 | 22.27 | 3.99 | 26 |
| 18 | 23.35 | 3.81 | 19 |
| 19 | 24.15 | 3.68 | 66 |
| 20 | 24.67 | 3.61 | 11 |
| 21 | 25.00 | 3.56 | 77 |
| 22 | 25.18 | 3.53 | 37 |
| 23 | 25.57 | 3.48 | 57 |
| 24 | 25.93 | 3.43 | 45 |
| 25 | 26.21 | 3.40 | 31 |
| 26 | 27.19 | 3.28 | 20 |
| 27 | 27.38 | 3.25 | 29 |

In some embodiments, each peak independently may include a variation of 0.1°, ±0.2°, or 0.30.

In one embodiment, the disclosure provides a crystal form of voruciclib oxalate, and/or a polymorph crystal form of voruciclib oxalate (Oxa1), characterized by an X-ray powder diffraction pattern including one or more peaks selected from:

| No | 2θ (°) | D (Å) | I (%) |
|---|---|---|---|
| 1 | 6.86 | 12.88 | 100 |
| 2 | 9.70 | 9.11 | 3 |
| 3 | 10.84 | 8.15 | 11 |
| 4 | 12.50 | 7.08 | 4 |
| 5 | 12.66 | 6.99 | 13 |
| 6 | 12.81 | 6.90 | 6 |
| 7 | 13.41 | 6.60 | 35 |
| 8 | 13.71 | 6.46 | 11 |
| 9 | 14.54 | 6.09 | 49 |
| 10 | 15.35 | 5.77 | 9 |
| 11 | 15.83 | 5.59 | 16 |
| 12 | 18.70 | 4.74 | 8 |
| 13 | 19.00 | 4.67 | 12 |
| 14 | 19.43 | 4.57 | 44 |
| 15 | 19.62 | 4.52 | 6 |
| 16 | 21.75 | 4.08 | 9 |
| 17 | 22.75 | 3.91 | 13 |
| 18 | 23.35 | 3.81 | 7 |
| 19 | 23.47 | 3.79 | 8 |
| 20 | 23.81 | 3.73 | 18 |
| 21 | 23.98 | 3.71 | 23 |
| 22 | 24.36 | 3.65 | 11 |
| 23 | 24.60 | 3.62 | 8 |
| 24 | 24.86 | 3.58 | 18 |
| 25 | 25.11 | 3.54 | 12 |
| 26 | 25.60 | 3.48 | 19 |
| 27 | 25.75 | 3.46 | 15 |
| 28 | 26.25 | 3.39 | 31 |

In some embodiments, each pea independently may include a variation of ±0.1°, ±0.2°, or ±0.30.

In one embodiment, the disclosure provides a crystal form of voruciclib phosphate, and/or a polymorph crystal form of voruciclib phosphate (Pho1), characterized by an X-ray powder diffraction pattern including one or more peaks selected from:

| No | 2θ (°) | D (Å) | I (%) |
|---|---|---|---|
| 1 | 4.93 | 17.92 | 31 |
| 2 | 6.79 | 13.01 | 61 |
| 3 | 9.35 | 9.45 | 22 |
| 4 | 10.58 | 8.35 | 12 |
| 5 | 10.91 | 8.10 | 52 |
| 6 | 12.64 | 7.00 | 37 |
| 7 | 13.35 | 6.63 | 23 |
| 8 | 13.58 | 6.51 | 7 |
| 9 | 14.81 | 5.98 | 100 |
| 10 | 15.60 | 5.68 | 28 |
| 11 | 17.18 | 5.16 | 14 |
| 12 | 17.52 | 5.06 | 15 |
| 13 | 18.32 | 4.84 | 14 |
| 14 | 18.78 | 4.72 | 25 |
| 15 | 19.34 | 4.59 | 10 |
| 16 | 19.64 | 4.52 | 13 |
| 17 | 19.78 | 4.49 | 23 |
| 18 | 22.02 | 4.03 | 28 |
| 19 | 23.20 | 3.83 | 16 |
| 20 | 23.67 | 3.76 | 36 |
| 21 | 24.00 | 3.70 | 45 |
| 22 | 24.71 | 3.60 | 35 |
| 23 | 25.21 | 3.53 | 20 |
| 24 | 25.39 | 3.51 | 19 |
| 25 | 26.55 | 3.35 | 23 |
| 26 | 27.22 | 3.27 | 13 |
| 27 | 28.07 | 3.18 | 11 |
| 28 | 29.90 | 2.99 | 15 |

In some embodiments, each peak independently may include a variation of ±0.1°, ±0.2°, or ±0.30.

In one embodiment, the disclosure provides a crystal form of voruciclib characterized by an X-ray powder diffraction pattern including one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38°±0.2°, 24.10°±0.2°, 24.98°±0.2°, 25.94°±0.2°, 27.26°±0.2°, 28.50°±0.20, and 32.82°±0.2° 2θ. In some embodiments, the X-ray diffraction pattern includes at least one peak, at least two peaks, at least three peaks, at least four peaks, at least five peaks, or the like, selected from the above group of peaks. In some embodiments, the crystal form includes voruciclib malonate. In some embodiments, the crystal form includes hydrated voruciclib malonate. In some embodiments, the crystal form includes anhydrous voruciclib malonate.

In one embodiment, the disclosure provides a crystal form of voruciclib characterized by an X-ray powder diffraction pattern including one or more peaks selected from 5.06°±0.2°, 6.42°±0.2°, 9.34°±0.2°, 10.14°±0.2°, 12.30°±0.2°, 13.66°±0.2°, 14.14°±0.2°, 15.82°±0.20, 17.020°±0.20, 19.740°±0.20, 20.380°±0.20, 21.820°±0.20, 22.660°±0.20, 24.620°±0.20, 25.78°±0.20, 26.58°±0.20, 28.66°±0.20, and 29.98°±0.2° 2θ. In some embodiments, the X-ray diffraction pattern includes at least one peak, at least two peaks, at least three peaks, at least four peaks, at least five peaks, or the like, selected from the above group of peaks. In some embodiments, the crystal form includes voruciclib dibenzoyl-tartrate. In some embodiments, the crystal form includes hydrated voruciclib dibenzoyl-tartrate. In some embodiments, the crystal form includes anhydrous voruciclib dibenzoyl-tartrate.

In one embodiment, the disclosure provides a crystal form of voruciclib characterized by an X-ray powder diffraction pattern including one or more peaks selected from 4.94°±0.2°, 6.78°±0.2°, 9.34°±0.2°, 10.94°±0.2°, 12.70°±0.2°, 13.38°±0.2°, 14.90°±0.2°, 15.66°±0.20, 17.540°±0.20, 18.820°±0.20, 22.020°±0.20, 23.980°±0.20, 24.780°±0.20, 25.300°±0.20, 26.66°±0.20, and 29.98°±0.2° 2θ. In some embodiments, the X-ray diffraction pattern includes at least one peak, at least two peaks, at least three peaks, at least four peaks, at least five peaks, or the like, selected from the above group of peaks. In some embodiments, the crystal form includes voruciclib phosphate. In some embodiments, the crystal form includes hydrated voruciclib phosphate. In some embodiments, the crystal form includes anhydrous voruciclib phosphate.

In one embodiment, the disclosure provides a crystal form of voruciclib characterized by an X-ray powder diffraction pattern including one or more peaks selected from 6.86°±0.2°, 12.66°±0.2°, 13.58°±0.2°, 14.74°±0.2°, 15.98°±0.2°, 19.38°±0.2°, 23.94°±0.2°, 24.78°±0.2°, and 25.94°±0.2° 2θ. In some embodiments, the X-ray diffraction pattern includes at least one peak, at least two peaks, at least three peaks, at least four peaks, at least five peaks, or the like, selected from the above group of peaks. In some embodiments, the crystal form includes voruciclib oxalate. In some embodiments, the crystal form includes hydrated voruciclib oxalate. In some embodiments, the crystal form includes anhydrous voruciclib oxalate.

In one embodiment, the disclosure provides a crystal form of voruciclib characterized by an X-ray powder diffraction pattern including one or more peaks selected from 9.02°±0.2°, 10.50°±0.2°, 11.06°±0.2°, 12.30°±0.2°, 12.82°±0.2°, 13.90°±0.2°, 14.82°±0.2°, 15.30°±0.2°, 15.94°±0.2°, 17.26°±0.2°, 19.34°±0.2°, 20.62°±0.2°, 22.18°±0.2°, 22.86°±0.2°, 24.58°±0.20, 25.42°±0.20, 25.86°±0.20, 27.38°±0.20, and 28.66°±0.2° 2θ. In some embodiments, the X-ray diffraction pattern includes at least one peak, at least two peaks, at least three peaks, at least four peaks, at least five peaks, or the like, selected from the above group of peaks. In some embodiments, the crystal form includes voruciclib napadisylate. In some embodiments, the crystal form includes hydrated voruciclib napadisylate. In some embodiments, the crystal form includes anhydrous voruciclib napadisylate.

In certain embodiments, the blood cancer is leukemia, such as acute myeloid leukemia (AML), chronic myeloid leukemia (CML), acute lymphocytic lymphoma (ALL), and chronic lymphocytic leukemia (CLL). In certain embodiments, the blood cancer is a non-Hodgkin lymphoma, such as B-cell or T-cell lymphoma. B-cell lymphomas include diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphomas, extranodal marginal zone B-cell lymphomas, nodal marginal zone B-cell lymphoma, splenic marginal zone B-cell lymphoma, Burkitt lymphoma, lymphoplasmacytic lymphoma, and primary central nervous system lymphoma. T-cell lymphomas include precursor T-lymphoblastic lymphoma, peripheral T-cell lymphomas, cutaneous T-cell lymphomas, adult T-cell lymphoma with subtypes: smoldering chronic, acute, and lymphoma, angioimmunoblastic T-cell lymphoma, extranodal natural killer/T-cell lymphoma, nasal type, enteropathy-associated intestinal T-cell lymphoma (EATL) with subtypes I and II, and anaplastic large cell lymphoma (ALCL). Combinations of the present disclosure may be used to treat a blood cancer described herein.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The disclosure provides methods of preventing, or reducing, a relapse of a cancer in a subject in need thereof. In certain embodiments, the term “prevent” or “preventing” as related to a disease or disorder may refer to a compound or combination that, in a statistical sample, reduces the occurrence of the disorder or condition in the treated sample relative to an untreated control sample, or delays the onset or reduces the severity of one or more symptoms of the disorder or condition relative to the untreated control sample. The method includes administering a combination therapy described herein to treat minimal residual disease, and/or as maintenance therapy, e.g., as a prolonged or extended therapy after cessation of another cancer treatment. For example, the combination therapy may be administered after cessation of another cancer therapy, such as chemotherapy, radiation therapy and/or surgery.

In certain aspects, a proteasome inhibitor may be combined or used in combination with a CDK inhibitor of the disclosure, e.g., a compound or salt of any one of Formulas I, Ia, or Ib. In eukaryotic cells, the ubiquitin (Ub)-proteasome pathway (UPS) involves Ub modification and subsequent degradation of protein substrates. UPS controls the levels of many cellular regulatory proteins, including transcription factors, cell cycle regulatory proteins and factors participating in a variety of cellular processes. The common feature of UPS pathway is that the highly conserved Ub is covalently attached to the target proteins through a series of enzymes, namely E1 Ub-activating enzyme, E2 Ub-conjugating enzyme and E3 Ub ligase. The E1 first activates Ub and transfers it to E2. From the E2 enzyme, the Ub is transferred directly to the target protein or indirectly through an E3 Ub ligase. The polyubiquitylated protein is recognized and degraded by 26S proteasome, a large complex with multiple proteolytic activities.

A combination therapy described herein can reduce the likelihood of metastasis in a subject in need thereof. In some embodiments, the metastasis is a solid tumor. In some embodiments, the metastasis is a liquid tumor. Cancers that are liquid tumors can be those that occur, for example, in blood, bone marrow, and lymph nodes, and can include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. In some embodiments, the condition treated by the methods described herein is metastasis of melanoma cells, prostate cancer cells, testicular cancer cells, breast cancer cells, brain cancer cells, pancreatic cancer cells, colon cancer cells, thyroid cancer cells, stomach cancer cells, lung cancer cells, ovarian cancer cells, Kaposi's sarcoma cells, skin cancer cells, renal cancer cells, head or neck cancer cells, throat cancer cells, squamous carcinoma cells, bladder cancer cells, osteosarcoma cells, cervical cancer cells, endometrial cancer cells, esophageal cancer cells, liver cancer cells, or kidney cancer cells.

The methods described herein can also be used for inhibiting progression of metastatic cancer tumors. Non-limiting examples of cancers include adrenocortical carcinoma, childhood adrenocortical carcinoma, AIDS-related cancers, anal cancer, appendix cancer, basal cell carcinoma, childhood basal cell carcinoma, bladder cancer, childhood bladder cancer, bone cancer, brain tumor, childhood astrocytomas, childhood brain stem glioma, childhood central nervous system atypical teratoid/rhabdoid tumor, childhood central nervous system embryonal tumors, childhood central nervous system germ cell tumors, childhood craniopharyngioma brain tumor, childhood ependymoma brain tumor, breast cancer, childhood bronchial tumors, carcinoid tumor, childhood carcinoid tumor, gastrointestinal carcinoid tumor, carcinoma of unknown primary, childhood carcinoma of unknown primary, childhood cardiac tumors, cervical cancer, childhood cervical cancer, childhood chordoma, chronic myeloproliferative disorders, colon cancer, colorectal cancer, childhood colorectal cancer, extrahepatic bile duct cancer, ductal carcinoma in situ (DCIS), endometrial cancer, esophageal cancer, childhood esophageal cancer, childhood esthesioneuroblastoma, eye cancer, malignant fibrous histiocytoma of bone, gallbladder cancer, gastric (stomach) cancer, childhood gastric cancer, gastrointestinal stromal tumors (GIST), childhood gastrointestinal stromal tumors (GIST), childhood extracranial germ cell tumor, extragonadal germ cell tumor, gestational trophoblastic tumor, glioma, head and neck cancer, childhood head and neck cancer, hepatocellular cancer, hypopharyngeal cancer, kidney cancer, renal cell kidney cancer, Wilms tumor, childhood kidney tumors, Langerhans cell histiocytosis, laryngeal cancer, childhood laryngeal cancer, leukemia, acute lymphoblastic leukemia (ALL), acute myeloid leukemia (AML), chronic lymphocytic leukemia (CLL), chronic myelogenous leukemia (cml), hairy cell leukemia, lip cancer, liver cancer (primary), childhood liver cancer (primary), lobular carcinoma in situ (LCIS), lung cancer, non-small cell lung cancer, small cell lung cancer, lymphoma, AIDS-related lymphoma, burkitt lymphoma, cutaneous t-cell lymphoma, Hodgkin lymphoma, non-Hodgkin lymphoma, primary central nervous system lymphoma (CNS), melanoma, childhood melanoma, intraocular melanoma, Merkel cell carcinoma, malignant mesothelioma, childhood malignant mesothelioma, metastatic squamous neck cancer with occult primary, midline tract carcinoma involving NUT gene, mouth cancer, childhood multiple endocrine neoplasia syndromes, mycosis fungoides, myelodysplastic syndromes, myelodysplastic neoplasms, myeloproliferative neoplasms, multiple myeloma, nasal cavity cancer, nasopharyngeal cancer, childhood nasopharyngeal cancer, neuroblastoma, oral cancer, childhood oral cancer, oropharyngeal cancer, ovarian cancer, childhood ovarian cancer, epithelial ovarian cancer, low malignant potential tumor ovarian cancer, pancreatic cancer, childhood pancreatic cancer, pancreatic neuroendocrine tumors (islet cell tumors), childhood papillomatosis, paraganglioma, paranasal sinus cancer, parathyroid cancer, penile cancer, pharyngeal cancer, pheochromocytoma, pituitary tumor, plasma cell neoplasm, childhood pleuropulmonary blastoma, prostate cancer, rectal cancer, renal pelvis transitional cell cancer, retinoblastoma, salivary gland cancer, childhood salivary gland cancer, Ewing sarcoma family of tumors, Kaposi Sarcoma, osteosarcoma, rhabdomyosarcoma, childhood rhabdomyosarcoma, soft tissue sarcoma, uterine sarcoma, Sezary syndrome, childhood skin cancer, nonmelanoma skin cancer, small intestine cancer, squamous cell carcinoma, childhood squamous cell carcinoma, testicular cancer, childhood testicular cancer, throat cancer, thymoma and thymic carcinoma, childhood thymoma and thymic carcinoma, thyroid cancer, childhood thyroid cancer, ureter transitional cell cancer, urethral cancer, endometrial uterine cancer, vaginal cancer, vulvar cancer, and Waldenström macroglobulinemia.

The combination therapies described herein may be used together with other therapies such as radiation therapy. Chemotherapy and radiotherapy treatment regimens can comprise a finite number of cycles of on-drug therapy followed by off-drug therapy, or comprise a finite timeframe in which the chemotherapy or radiotherapy is administered. The protocols can be determined by clinical trials, drug labels, and clinical staff in conjunction with the subject to be treated. The number of cycles of a chemotherapy or radiotherapy or the total length of time of a chemotherapy or radiotherapy regimen can vary depending on the subject's response to the cancer therapy. A pharmaceutical agent described herein can be administered after the treatment regimen of chemotherapy or radiotherapy has been completed.

In some aspects, the combinations described herein can be utilized to treat a subject in need thereof. In some cases, the subject to be treated by methods and compositions disclosed herein can be a human subject. A subject to be treated by methods and compositions disclosed herein can be a non-human animal. Non-limiting examples of non-human animals can include a non-human primate, a livestock animal, a domestic pet, and a laboratory animal.

In certain embodiments, the combination therapies described herein may be administered as separate agents or may be combined into a single pharmaceutical composition.

In certain embodiments, the disclosure provides a pharmaceutical composition, e.g., for oral or parenteral administration, comprising a compound or salt of Formula I, Ia, or Ib. In some aspects, the pharmaceutical composition comprises a compound or salt of Formula I, Ia, or Ib in an amount of at least about 1 mg to about 1000 mg, from about 100 mg to about 400 mg, from about 100 mg to about 200 mg, from about 200 mg to about 400 mg, or from about 250 mg to about 350 mg. For example, a pharmaceutical composition of the disclosure may comprise about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg of a compound of Formula I, Ia, or Ib. For a compound described herein, e.g., a compound of Formula Ib, formulated into a pharmaceutical composition in the form of a salt, the amount of the compound may reflect the free base weight and not the weight of the salt form. In certain embodiments, the pharmaceutical composition of the compound or salt of Formula I, Ia, or Ib does not include an additional anticancer agent. In certain embodiments, the pharmaceutical composition includes an additional anticancer agent.

A therapeutically effective amount of a compound of the disclosure, e.g., a compound or salt of Formula I, Ia, or Ib, can be expressed as mg of the compound per kg of subject body mass. In some instances, a dose of a therapeutically effective amount may be at least about 0.1 mg/kg to about 20 mg/kg, for example, about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, or about 20 mg/kg. For a compound described herein, e.g., a compound of Formula Ib, formulated into a pharmaceutical composition in the form of a salt, the therapeutically effective amount of the compound may reflect the free base weight and not the weight of the salt form.

In certain embodiments, the disclosure provides a pharmaceutical composition, e.g., for oral or parenteral administration, comprising an additional therapeutic agent, e.g., anticancer agent, in an amount of at least about 1 mg to about 1000 mg, from about 100 mg to about 1000 mg, from about 100 mg to about 800 mg, from about 200 mg to about 800 mg, or from about 300 mg to about 8000 mg. For example, a pharmaceutical composition of the disclosure may comprise about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, about 500 mg, about 520 mg, about 540 mg, about 560 mg, about 580 mg, about 600 mg, about 620 mg, about 640 mg, about 660 mg, about 680 mg, about 700 mg, about 720 mg, about 740 mg, about 760 mg, about 780 mg, about 800 mg, about 820 mg, about 840 mg, about 860 mg, about 880 mg, about 900 mg, about 920 mg, about 940 mg, about 960 mg, about 980 mg, or about 1000 mg of an additional therapeutic agent, e.g., anticancer agent.

The amounts of the solid form of voruciclib, including any voruciclib free base polymorph described herein, or any voruciclib salt polymorph described herein, administered will be dependent on the mammal being treated, the severity of the disorder or condition, the rate of administration, the disposition of the compounds and the discretion of the prescribing physician. However, an effective dosage is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect, for example by dividing such larger doses into several small doses for administration throughout the day.

In selected embodiments, a solid form of voruciclib, including any voruciclib free base polymorph described herein, or any voruciclib salt polymorph described herein, is administered in a single dose. Typically, such administration will be by injection, for example by intravenous injection, in order to introduce the active pharmaceutical ingredients quickly. However, other routes may be used as appropriate. A single dose of a solid form of voruciclib, including any voruciclib free base polymorph described herein, or any voruciclib salt polymorph described herein, may also be used for treatment of an acute condition.

In selected embodiments, a solid form of voruciclib, including any voruciclib free base polymorph described herein, or any voruciclib salt polymorph described herein, is administered in multiple doses. Dosing may be about once, twice, three times, four times, five times, six times, or more than six times per day. Dosing may be about once a month, once every two weeks, once a week, or once every other day. In other embodiments, a solid form of voruciclib, including any voruciclib free base polymorph described herein, or any voruciclib salt polymorph described herein, is administered about once per day to about 6 times per day. In another embodiment the administration of the solid forms of voruciclib, including any voruciclib free base polymorph described herein, or any voruciclib salt polymorph described herein, continues for less than about 7 days. In yet another embodiment the administration continues for more than about 6, 10, 14, 28 days, two months, six months, or one year. In some cases, continuous dosing is achieved and maintained as long as necessary. In an embodiment, the solid form of voruciclib is selected from voruciclib malonate, voruciclib dibenzoyl-tartrate, voruciclib phosphate, voruciclib oxalate, and voruciclib napadisylate, each as described herein.

Administration of the active pharmaceutical ingredients of the disclosure may continue as long as necessary. In selected embodiments, a solid form of voruciclib, including any voruciclib free base polymorph described herein, or any voruciclib salt polymorph described herein, is administered for more than 1, 2, 3, 4, 5, 6, 7, 14, or 28 days. In some embodiments, the solid forms of voruciclib, including any voruciclib free base polymorph described herein, or any voruciclib salt polymorph described herein, are administered for less than 28, 14, 7, 6, 5, 4, 3, 2, or 1 day. In selected embodiments, a solid form of voruciclib, including any voruciclib free base polymorph described herein, or any voruciclib salt polymorph described herein, is administered chronically on an ongoing basis—e.g., for the treatment of chronic effects. In an embodiment, the solid form of voruciclib, in any of the foregoing embodiments is selected from voruciclib malonate, voruciclib dibenzoyl-tartrate, voruciclib phosphate, voruciclib oxalate, and voruciclib napadisylate, each as described herein.

In some embodiments, an effective dosage of voruciclib, including any voruciclib free base polymorph described herein, or any voruciclib salt polymorph described herein, is in the range of about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of a solid form of voruciclib, including any voruciclib free base polymorph described herein, or any voruciclib salt polymorph described herein, is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In some embodiments, an effective dosage of a solid form of voruciclib, including any voruciclib free base polymorph described herein, or any voruciclib salt polymorph described herein, is 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg. In some embodiments, an effective daily dosage of a solid form of voruciclib free base, or its calculated salt and/or salt polymorph equivalent amount, is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg. In some embodiments, an effective daily dosage of a solid form of voruciclib free base, or its calculated salt and/or salt polymorph equivalent amount, is a tolerable dosage determined by one skilled in the art, for example less than about 25 mg, less than about 50 mg, less than about 75 mg, less than about 100 mg, less than about 125 mg, less than about 150 mg, less than about 175 mg, less than about 200 mg, less than about 225 mg, less than about 250 mg, less than about 275 mg, less than about 300 mg, less than about 325 mg, less than about 350 mg, less than about 375 mg, less than about 400 mg, less than about 425 mg, less than about 450 mg, less than about 475 mg, or less than about 500 mg. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein. In some embodiments, a dosage refers to a daily dosage. As one skilled in the art understands, exact amounts and/or dosages can be corrected for drug substance assay (free base on the anhydrous, solvent free basis). Salt conversion factor can also be derived by one skilled in the art. For example, the malonate salt conversion factor is 0.8187, such that 61.07 mg of voruciclib malonate equals 50 mg of voruciclib free base and 121.14 mg of voruciclib malonate equals 100 mg of voruciclib free base.

In some embodiments, an effective dosage of voruciclib, including any voruciclib free base polymorph described herein, or any voruciclib salt polymorph described herein, is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of a solid form of voruciclib, including any voruciclib free base polymorph described herein, or any voruciclib salt polymorph described herein, is about 0.35 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, a solid form of voruciclib, including any voruciclib free base polymorph described herein, any voruciclib salt polymorph described herein, is administered at a dosage of 10 to 400 mg once daily (QD), including a dosage of 5 mg, 10 mg, 12.5 mg, 25 mg, 50 mg, 75 mg, 100 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg once daily (QD). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, a solid form of voruciclib, including any voruciclib free base polymorph described herein, or any voruciclib salt polymorph described herein, is administered at a dosage of 10 to 400 mg BID, including a dosage of 5 mg, 10 mg, 12.5 mg, 25 mg, 50 mg, 75 mg, 100 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg BID. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, a solid form of voruciclib, including any voruciclib free base polymorph described herein, or any voruciclib salt polymorph described herein, is administered at a dosage of 10 to 400 mg TID, including a dosage of 5 mg, 10 mg, 12.5 mg, 25 mg, 50 mg, 75 mg, 100 mg, 150 mg, 175 mg, 200 mg, 225 mg, 250 mg, 275 mg, 300 mg, 325 mg, 350 mg, 375 mg, 400 mg, 425 mg, 450 mg, 475 mg, and 500 mg TID. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

An effective amount of a solid form of voruciclib, including any voruciclib free base polymorph described herein, or any voruciclib salt polymorph described herein, may be administered in either single or multiple doses by any of the accepted modes of administration of active pharmaceutical ingredients having similar utilities, including rectal, buccal, intranasal and transdermal routes, by intra-arterial injection, intravenously, intraperitoneally, parenterally, intramuscularly, subcutaneously, orally, topically, or as an inhalant.

The compositions and methods described herein can be used to overcome the effects of acid reducing agents. Acid-reducing agents can greatly limit the exposure of weakly acidic drugs in mammals. Smelick, et al., Mol. Pharmaceutics 2013, 10, 4055-4062. Acid reducing agents include proton pump inhibitors, such as omeprazole, esomeprazole, lansoprazole, dexlansoprazole, pantoprazole, rabeprazole, and ilaprazole; H2 receptor antagonists, such as cimetidine, ranitidine, and famotidine; and antacids such as bicarbonates, carbonates, and hydroxides of aluminum, calcium, magnesium, potassium, and sodium, as well as mixtures of antacids with agents targeting mechanisms of gastric secretion. Overcoming the effects of acid reducing agents is a significant issue in the treatment of patients with cancer, inflammatory diseases, immune diseases, and autoimmune diseases, since these patients are commonly co-administered acid reducing agents for gastric irritation that often accompanies their conditions, because acid reducing agents are some of the most commonly prescribed medications in North America and Western Europe. Most recently approved oral cancer therapeutics have pH-dependent solubility and thus a potential drug-drug interaction with regards to acid reducing agents. In cancer patients, it is estimated that 20-33% of all patients are using some form of acid-reducing agent. In particular cancers, such as pancreatic cancer or gastrointestinal cancers, acid reducing agent use is as high as 60-80% of patients. Smelick, et al., Mol. Pharmaceutics 2013, 10, 4055-4062.

In an embodiment, a pharmaceutical composition comprises voruciclib, including any voruciclib free base polymorph described herein, or any voruciclib salt polymorph described herein, and an acidulant. In an embodiment, a pharmaceutical composition comprises voruciclib, including any voruciclib free base polymorph described herein, or any voruciclib salt polymorph described herein, and an acidulant selected from the group consisting of fumaric acid, tartaric acid, ascorbic acid, alginic acid, sodium alginate, potassium alginate, and Carbopol 971P (carboxypolymethylene). In an embodiment, a pharmaceutical composition comprises voruciclib, including any voruciclib free base polymorph described herein, or any voruciclib salt polymorph described herein, and an acidulant selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, Protacid F 120 NM, Protacid AR 1112 (also known as Kelacid NF), Carbomer 941 (polyacrylic acid), and Carbopol 971P (carboxypolymethylene). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein. In an embodiment, the acidulant is extragranular. In an embodiment, the acidulant is intragranular.

Alginic acid is a polysaccharide copolymer, β-D-mannuronic acid (M) and α-L-guluronic acid (G) linked by 1-4 glycosidic bonds. In an embodiment, a pharmaceutical composition comprises voruciclib, including any voruciclib free base polymorph described herein, or any voruciclib salt polymorph described herein, and an acidulant that is an alginic acid or salt thereof, wherein the alginic acid or salt thereof exhibits an M/G ratio selected from the group consisting of between 0.1 and 0.5, between 0.2 and 0.6, between 0.3 and 0.7, between 0.4 and 0.8, between 0.5 and 0.9, between 0.6 and 1.0, between 0.7 and 1.1, between 0.8 and 1.2, between 0.9 and 1.3, between 1.0 and 1.4, between 1.1 and 1.5, between 1.2 and 1.6, between 1.3 and 1.7, between 1.4 and 1.8, between 1.5 and 1.9, between 1.6 and 2.0, between 1.7 and 2.1, between 1.8 and 2.2, between 1.9 and 2.3, between 2.0 and 2.4, and between 2.1 and 2.5. In an embodiment, a pharmaceutical composition comprises voruciclib, including any voruciclib free base polymorph described herein, or any voruciclib salt polymorph described herein, and an acidulant that is an alginic acid or salt thereof, wherein the alginic acid or salt thereof exhibits an M/G ratio selected from the group consisting of less than 0.5, less than 1.0, less than 1.5, less than 2.0, and less than 2.5. In an embodiment, a pharmaceutical composition comprises voruciclib, including any voruciclib free base polymorph described herein, or any voruciclib salt polymorph described herein, and an acidulant that is an alginic acid or salt thereof, wherein the alginic acid or salt thereof exhibits an M/G ratio selected from the group consisting of greater than 0.5, greater than 1.0, greater than 1.5, greater than 2.0, and greater than 2.5. In an embodiment, a pharmaceutical composition comprises voruciclib, including any voruciclib free base polymorph described herein, or any voruciclib salt polymorph described herein, and an acidulant that is an alginic acid or salt thereof, wherein the alginic acid or salt thereof exhibits an M/G ratio selected from the group consisting of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, and 2.5. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

M/G ratio, as well as the fraction of M and G groups, the fractions of MM and GG “diads,” the fractions of “triads” (e.g., MGG), and the fractions of larger sequences of M and G groups, may be determined by methods known to those of ordinary skill in the art, including nuclear magnetic resonance (NMR) spectroscopy (with or without digestion) and mass spectrometry. Larsen, et al., Carbohydr. Res., 2003, 338, 2325-2336.

In an embodiment, a pharmaceutical composition comprises voruciclib, including any voruciclib free base polymorph described herein, or any voruciclib salt polymorph described herein, and an acidulant in a concentration (% mass) selected from the group consisting of between 1% and 5%, between 5% and 10%, between 10% and 15%, between 15% and 20%, between 20% and 25%, between 25% and 30%, and between 30% and 35%. In an embodiment, a pharmaceutical composition comprises voruciclib, including any voruciclib free base polymorph described herein, or any voruciclib salt polymorph described herein, and an acidulant in a concentration (% mass) selected from the group consisting of between 1% and 5%, between 5% and 10%, between 10% and 15%, between 15% and 20%, between 20% and 25%, between 25% and 30%, and between 30% and 35%, wherein the acidulant is selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, sodium alginate, potassium alginate, Protacid F 120 NM, Protacid AR 1112 (also known as Kelacid NF), and Carbopol 971P (carboxypolymethylene). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In an embodiment, a pharmaceutical composition comprises voruciclib, including any voruciclib free base polymorph described herein, or any voruciclib salt polymorph described herein, and an acidulant in a concentration (% mass) selected from the group consisting of less than 1%, less than 5%, less than 10%, less than 15%, less than 20%, less than 25%, less than 30%, and less than 35%. In an embodiment, a pharmaceutical composition comprises voruciclib, including any voruciclib free base polymorph described herein, or any voruciclib salt polymorph described herein, and an acidulant in a concentration (% mass) selected from the group consisting of less than 1%, less than 5%, less than 10%, less than 15%, less than 20%, less than 25%, less than 30%, and less than 35%, wherein the acidulant is selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, sodium alginate, potassium alginate, Protacid F 120 NM, Protacid AR 1112 (also known as Kelacid NF), and Carbopol 971P (carboxypolymethylene). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In an embodiment, a pharmaceutical composition comprises voruciclib, including any voruciclib free base polymorph described herein, or any voruciclib salt polymorph described herein, and an acidulant in a concentration (% mass) selected from the group consisting of greater than 1%, greater than 5%, greater than 10%, greater than 15%, greater than 20%, greater than 25%, greater than 30%, and greater than 35%. In an embodiment, a pharmaceutical composition comprises voruciclib, including any voruciclib free base polymorph described herein, or any voruciclib salt polymorph described herein, and an acidulant in a concentration (% mass) selected from the group consisting of greater than 1%, greater than 5%, greater than 10%, greater than 15%, greater than 20%, greater than 25%, greater than 30%, and greater than 35%, wherein the acidulant is selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, sodium alginate, potassium alginate, Protacid F 120 NM, Protacid AR 1112 (also known as Kelacid NF), and Carbopol 971P (carboxypolymethylene). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In an embodiment, a pharmaceutical composition comprises voruciclib, including any voruciclib free base polymorph described herein, or any voruciclib salt polymorph described herein, and an acidulant in a concentration (% mass) selected from the group consisting of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, and about 40%. In an embodiment, a pharmaceutical composition comprises voruciclib, including any voruciclib free base polymorph described herein, or any voruciclib salt polymorph described herein, and an acidulant in a concentration (% mass) selected from the group consisting of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, and about 40%, wherein the acidulant is selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, sodium alginate, potassium alginate, Protacid F 120 NM, Protacid AR 1112 (also known as Kelacid NF), and Carbopol 971P (carboxypolymethylene). In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In an embodiment, a pharmaceutical composition comprises voruciclib, including any voruciclib free base polymorph described herein, or any voruciclib salt polymorph described herein, and an extragranular acidulant, wherein the extragranular acidulant is selected from the group consisting of fumaric acid, succinic acid, D-tartaric acid, L-tartaric acid, racemic tartaric acid, ascorbic acid, isoascorbic acid (also known as erythorbic acid and D-araboascorbic acid), alginic acid, sodium alginate, potassium alginate, Protacid F 120 NM, Protacid AR 1112 (also known as Kelacid NF), and Carbopol 971P (carboxypolymethylene), and combinations thereof. In an embodiment, a pharmaceutical composition comprises voruciclib, including any voruciclib free base polymorph described herein, or any voruciclib salt polymorph described herein, and an extragranular acidulant, wherein the extragranular acidulant is fumaric acid at a concentration of between about 15% to about 33% by weight. In an embodiment, a pharmaceutical composition comprises voruciclib, including any voruciclib free base polymorph described herein, or any voruciclib salt polymorph described herein, and an extragranular acidulant, wherein the extragranular acidulant is alginic acid or a salt thereof (such as sodium alginate or potassium alginate) at a concentration of between about 5% to about 33% by weight. In an embodiment, a pharmaceutical composition comprises voruciclib, including any voruciclib free base polymorph described herein, or any voruciclib salt polymorph described herein, and an extragranular acidulant, wherein the extragranular acidulant is L-tartaric acid at a concentration of between about 25% to about 33% by weight. In an embodiment, a pharmaceutical composition comprises voruciclib, including any voruciclib free base polymorph described herein, or any voruciclib salt polymorph described herein, and an extragranular acidulant, wherein the extragranular acidulant is ascorbic acid at a concentration of between about 20% to about 50% by weight and Carbopol 971P (carboxypolymethylene) at a concentration of between about 2.5% to about 10% by weight. In an embodiment, a pharmaceutical composition comprises voruciclib, including any voruciclib free base polymorph described herein, or any voruciclib salt polymorph described herein, and an extragranular acidulant, wherein the extragranular acidulant is fumaric acid at a concentration of between about 5% to about 15% by weight and alginic acid or a salt thereof at a concentration of about 15% to about 33% by weight. In an embodiment, a pharmaceutical composition comprises voruciclib, including any voruciclib free base polymorph described herein, or any voruciclib salt polymorph described herein, and an extragranular acidulant, wherein the extragranular acidulant is L-tartaric acid at a concentration of between about 5% to 15% by weight and alginic acid at a concentration of between about 15% to about 33% by weight.

In an embodiment, a pharmaceutical composition comprises voruciclib, including any voruciclib free base polymorph described herein, or any voruciclib salt polymorph described herein, and an acidulant, wherein the acidulant is selected from the group consisting of fumaric acid, maleic acid, phosphoric acid, L-tartaric acid, citric acid, gentisic acid, oxalic acid, and sulfuric acid. In an embodiment, a pharmaceutical composition comprises voruciclib, including any voruciclib free base polymorph described herein, or any voruciclib salt polymorph described herein, and an acidulant, wherein the acidulant is selected from the group consisting of fumaric acid, maleic acid, phosphoric acid, L-tartaric acid, citric acid, gentisic acid, oxalic acid, and sulfuric acid, and wherein the acidulant is a salt counterion included in any crystalline form described herein.

In an embodiment, in addition to an acidulant, a pharmaceutical composition includes an excipient to prolong the exposure of voruciclib, including any voruciclib free base polymorph described herein, or any voruciclib salt polymorph described herein, to the acidic microenvironment. In an embodiment, this excipient is a polymer of natural, synthetic or semisynthetic origins. The polymer may contain acidic, anionic, or non-ionic monomers, oligomers or polymers or a mixture of acidic, anionic and non-ionic monomers or copolymers. In one version the excipient is selected from the group consisting of hydroxypropylmethylcellulose, low substituted hydroxypropylcellulose, hydroxypropylcellulose, tocopherol polyethyleneoxide succinate (D-α-tocopherol polyethylene glycol succinate, TPGS, or vitamin E TPGS), methylcellulose, carboxymethylcellulose, sodium carboxymethylcellulose, methylacrylate, ethylacrylate, co-polymers of methyl and ethyl acrylate, hydroxypropylmethylcellulose acetate succinate, gelatin, maize starch, pea starch, modified maize starch, potato starch, modified potato starch, sodium starch glycolate, croscarmellose, crospovidone, copovidone, polyethylene glycol, polypropylene glycol, polyethylene and polypropylene glycol copolymers, polyvinylalcohol, polyvinylalcohol and polyethylene oxide copolymers. Copolymers of the foregoing polymers, where applicable, may also be used. Copolymers may be block, branched or terminal copolymers. In an embodiment, the polymer exhibits swelling, binding, or gelling properties that inhibit the disintegration, dissolution, and erosion of the pharmaceutical composition in order to prolong dissolution or to increase total dissolution. In an embodiment, the inclusion of the polymer increases dissolution rate and extent of dissolution over the use of an acidulant alone. The swelling, binding or gelling properties are pH-dependent in one embodiment, wherein the polymer swells, binds, or gels at one pH or range of pH in a different manner than at another pH. In one embodiment this may decrease dissolution at a lower pH than at a higher pH or vice versa. In another embodiment this leads to similar dissolution of voruciclib, including any voruciclib free base polymorph described herein, or any voruciclib salt polymorph described herein, in acidic, neutral or basic pH. This leads to similar plasma exposure independent of stomach pH.

The dissolution profile of a formulation containing one or more swelling, gelling, or binding excipients may exhibit a zero, first, or second differential rate order at one or more pH value or a mixture of different rate orders at different pH values. In an embodiment, a pharmaceutical composition will provide a constant level of drug into the gastrointestinal tract of a mammal by dissolution. Where voruciclib, including any voruciclib free base polymorph described herein, or any voruciclib salt polymorph described herein, is absorbed, this leads to a sustained plasma level of drug over a period, delays the tₘₐₓ, and reduces the cₘₐₓ of an equivalent dose of an immediate release formulation voruciclib, including any voruciclib free base polymorph described herein, or any voruciclib salt polymorph described herein. In another embodiment this leads to similar exposure in a mammal regardless of stomach pH.

The pharmaceutical compositions described herein can be used in a method for treating diseases. In preferred embodiments, they are for use in treating hyperproliferative disorders. They may also be used in treating other disorders as described herein and in the following paragraphs.

In some embodiments, the disclosure provides a method of treating a hyperproliferative disorder in a mammal that comprises administering to the mammal a therapeutically effective amount of a crystalline solid form of voruciclib, including any voruciclib free base polymorph described herein, or any voruciclib salt polymorph described herein, or a pharmaceutical composition comprising a crystalline solid form of voruciclib, including any voruciclib free base polymorph described herein, or any voruciclib salt polymorph described herein, as described herein. In preferred embodiments, the mammal is a human. In some embodiments, the hyperproliferative disorder is cancer. In preferred embodiments, the cancer is selected from the group consisting of chronic lymphocytic leukemia, non-Hodgkin's lymphoma, diffuse large B-cell lymphoma, mantle cell lymphoma, follicular lymphoma, and Waldenström's macroglobulinemia. In preferred embodiments, the cancer is selected from the group consisting of non-Hodgkin's lymphomas (such as diffuse large B-cell lymphoma), acute myeloid leukemia, thymus, brain, lung, squamous cell, skin, eye, retinoblastoma, intraocular melanoma, oral cavity and oropharyngeal, bladder, gastric, stomach, pancreatic, bladder, breast, cervical, head, neck, renal, kidney, liver, ovarian, prostate, colorectal, bone (e.g., metastatic bone), esophageal, testicular, gynecological, thyroid, CNS, PNS, AIDS-related (e.g., lymphoma and Kaposi's sarcoma), viral-induced cancers such as cervical carcinoma (human papillomavirus), B-cell lymphoproliferative disease and nasopharyngeal carcinoma (Epstein-Barr virus), Kaposi's sarcoma and primary effusion lymphomas (Kaposi's sarcoma herpesvirus), hepatocellular carcinoma (hepatitis B and hepatitis C viruses), and T-cell leukemias (Human T-cell leukemia virus-1), B cell acute lymphoblastic leukemia, Burkitt's leukemia, juvenile myelomonocytic leukemia, hairy cell leukemia, Hodgkin's disease, multiple myeloma, mast cell leukemia, and mastocytosis. In selected embodiments, the method relates to the treatment of a non-cancerous hyperproliferative disorder such as benign hyperplasia of the skin (e.g., psoriasis), restenosis, or prostate conditions (e.g., benign prostatic hypertrophy (BPH)). In some embodiments, the hyperproliferative disorder is an inflammatory, immune, or autoimmune disorder. In some embodiments, the hyperproliferative disorder is selected from the group consisting of tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spondylitis, Crohn's disease, lupus, and lupus nephritis. In an embodiment, the solid form of voruciclib, including any voruciclib free base polymorph described herein, or any voruciclib salt polymorph described herein, in any of the foregoing embodiments is selected from voruciclib malonate, voruciclib dibenzoyl-tartrate, voruciclib phosphate, voruciclib oxalate, and voruciclib napadisylate, each as described herein.

In an embodiment, the method of any of the foregoing embodiments further includes the step of administering an acid reducing agent to the mammal. In an embodiment, the acid reducing agent is selected from the group consisting of proton pump inhibitors, such as omeprazole, esomeprazole, lansoprazole, dexlansoprazole, pantoprazole, rabeprazole, and ilaprazole; H₂ receptor antagonists, such as cimetidine, ranitidine, and famotidine; and antacids such as bicarbonates, carbonates, and hydroxides of aluminum, calcium, magnesium, potassium, and sodium.

In some embodiments, the disclosure provides pharmaceutical compositions of a solid form of voruciclib, including any voruciclib free base polymorph described herein, or any voruciclib salt polymorph described herein, for use in the treatment of cancers such as thymus cancer, brain cancer (e.g., glioma), lung cancer, squamous cell cancer, skin cancer (e.g., melanoma), eye cancer, retinoblastoma cancer, intraocular melanoma cancer, oral cavity cancer, oropharyngeal cancer, bladder cancer, gastric cancer, stomach cancer, pancreatic cancer, bladder cancer, breast cancer, cervical cancer, head and neck cancer, renal cancer, kidney cancer, liver cancer, ovarian cancer, prostate cancer, colorectal cancer, colon cancer, esophageal cancer, testicular cancer, gynecological cancer, ovarian cancer, thyroid cancer, CNS cancer, PNS cancer, AIDS-related cancer (e.g., lymphoma and Kaposi's sarcoma), viral-induced cancer, and epidermoid cancer. In some embodiments, the disclosure provides pharmaceutical compositions of a solid form of voruciclib, including any voruciclib free base polymorph described herein, or any voruciclib salt polymorph described herein, for the treatment of a non-cancerous hyperproliferative disorder such as benign hyperplasia of the skin (e.g., psoriasis), restenosis, or prostate (e.g., benign prostatic hypertrophy (BPH)). In some embodiments, the disclosure provides pharmaceutical compositions of a solid form of voruciclib, including any voruciclib free base polymorph described herein, or any voruciclib salt polymorph described herein, for use in the treatment of disorders such as myeloproliferative disorders (MPDs), myeloproliferative neoplasms, polycythemia vera (PV), essential thrombocythemia (ET), primary myelofibrosis (PMF), myelodysplastic syndrome, chronic myelogenous leukemia (BCR-ABL1-positive), chronic neutrophilic leukemia, chronic eosinophilic leukemia, or mastocytosis. The disclosure also provides compositions for use in treating a disease related to vasculogenesis or angiogenesis in a mammal which can manifest as tumor angiogenesis, chronic inflammatory disease such as rheumatoid arthritis, inflammatory bowel disease, atherosclerosis, skin diseases such as psoriasis, eczema, and scleroderma, diabetes, diabetic retinopathy, retinopathy of prematurity, age-related macular degeneration, and hemangioma. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, the disclosure provides a method of treating a solid tumor cancer with a composition including a solid form of voruciclib, including any voruciclib free base polymorph described herein, or any voruciclib salt polymorph described herein. In some embodiments, the disclosure provides a method of treating pancreatic cancer, breast cancer, ovarian cancer, melanoma, lung cancer, squamous cell carcinoma including head and neck cancer, or a blood cancer. In an embodiment, the disclosure provides a method for treating pancreatic cancer, breast cancer, ovarian cancer, melanoma, lung cancer, head and neck cancer, colorectal cancer, or a blood cancer using a combination of a solid form of voruciclib, including any voruciclib free base polymorph described herein, or any voruciclib salt polymorph described herein, and a second agent selected from the group consisting of bendamustine, venetoclax, vemurafenib, abraxane, enasidenib, pomalidomide, lenalidomide, azacitidine, decitabine, a hypomethylating agent, gemcitabine, albumin-bound paclitaxel, rituximab, obinutuzumab, ofatumumab, pembrolizumab, nivolumab, durvalumab, avelumab, atezolizumab, bortezomib, marizomib, ixazomib, disulfiram, epigallocatechin-3-gallate, salinosporamide A, carfilzomib, ONX 0912, CEP-18770, MLN9708, epoxomicin, or MG13. In an embodiment, the disclosure provides a method for treating pancreatic cancer, breast cancer, ovarian cancer, melanoma, lung cancer, head and neck cancer, colorectal cancer, or a blood cancer using a combination of a CDK inhibitor and bendamustine, venetoclax, vemurafenib, abraxane, enasidenib, pomalidomide, lenalidomide, azacitidine, decitabine, a hypomethylating agent, gemcitabine, albumin-bound paclitaxel, rituximab, obinutuzumab, ofatumumab, pembrolizumab, nivolumab, durvalumab, avelumab, atezolizumab, For certain methods described herein, the proteasome inhibitor is selected from bortezomib, marizomib, ixazomib, disulfiram, epigallocatechin-3-gallate, salinosporamide A, carfilzomib, ONX 0912, CEP-18770, MLN9708, epoxomicin, or MG13, wherein the CDK inhibitor is a solid form of voruciclib, including any voruciclib free base polymorph described herein, or any voruciclib salt polymorph described herein. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, the disclosure provides a method of treating a solid tumor cancer with a composition including a solid form of voruciclib, including any voruciclib free base polymorph described herein, or any voruciclib salt polymorph described herein. In some embodiments, the disclosure provides a method of treating pancreatic cancer, breast cancer, ovarian cancer, melanoma, lung cancer, squamous cell carcinoma including head and neck cancer. In an embodiment, the disclosure provides a method for treating pancreatic cancer, breast cancer, ovarian cancer, melanoma, lung cancer, head and neck cancer, and colorectal cancer using a solid form of voruciclib, including any voruciclib free base polymorph described herein, or any voruciclib salt polymorph described herein. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, the disclosure relates to a method of treating an inflammatory, immune, or autoimmune disorder in a mammal with a composition including a solid form of voruciclib, including any voruciclib free base polymorph described herein, or any voruciclib salt polymorph described herein. In selected embodiments, the disclosure also relates to a method of treating a disease with a composition including a solid form of voruciclib, including any voruciclib free base polymorph described herein, or any voruciclib salt polymorph described herein, wherein the disease is selected from the group consisting of tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma and melanoma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spondylitis, Crohn's Disease, lupus, and lupus nephritis. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, the disclosure relates to a method of treating a hyperproliferative disorder in a mammal with a composition including a solid form of voruciclib, including any voruciclib free base polymorph described herein, or any voruciclib salt polymorph described herein, wherein the hyperproliferative disorder is a B cell hematological malignancy selected from the group consisting of chronic lymphocytic leukemia (CLL), small lymphocytic leukemia (SLL), non-Hodgkin's lymphoma (NHL), diffuse large B cell lymphoma (DLBCL), follicular lymphoma (FL), mantle cell lymphoma (MCL), Hodgkin's lymphoma, B cell acute lymphoblastic leukemia (B-ALL), Burkitt's lymphoma, Waldenström's macroglobulinemia (WM), Burkitt's lymphoma, multiple myeloma, myelodysplastic syndromes, or myelofibrosis. In some embodiments, the disclosure relates to a method of treating a hyperproliferative disorder in a mammal with a composition including a solid form of voruciclib, including any voruciclib free base polymorph described herein, or any voruciclib salt polymorph described herein, wherein the hyperproliferative disorder is selected from the group consisting of chronic myelocytic leukemia, acute myeloid leukemia, DLBCL (including activated B-cell (ABC) and germinal center B-cell (GCB) subtypes), follicle center lymphoma, Hodgkin's disease, multiple myeloma, indolent non-Hodgkin's lymphoma, and mature B-cell ALL. In an embodiment, the solid form of voruciclib in any of the foregoing embodiments is selected from voruciclib malonate, voruciclib dibenzoyl-tartrate, voruciclib phosphate, voruciclib oxalate, and voruciclib napadisylate, each as described herein.

In some embodiments, the hyperproliferative disorder is a subtype of CLL. A number of subtypes of CLL have been characterized. CLL is often classified for immunoglobulin heavy-chain variable-region (IgVH) mutational status in leukemic cells. R. N. Damle, et al., Blood 1999, 94, 1840-47; T. J. Hamblin, et al., Blood 1999, 94, 1848-54. Patients with IgV_{H} mutations generally survive longer than patients without IgV_{H} mutations. ZAP70 expression (positive or negative) is also used to characterize CLL. L. Z. Rassenti, et al., N. Engl. J. Med. 2004, 351, 893-901. The methylation of ZAP-70 at CpG3 is also used to characterize CLL, for example by pyrosequencing. R. Claus, et al., J. Clin. Oncol. 2012, 30, 2483-91; J. A. Woyach, et al., Blood 2014, 123, 1810-17. CLL is also classified by stage of disease under the Binet or Rai criteria. J. L. Binet, et al., Cancer 1977, 40, 855-64; K. R. Rai, T. Han, Hematol. Oncol. Clin. North Am. 1990, 4, 447-56. Other common mutations, such as 11q deletion, 13q deletion, and 17p deletion can be assessed using well-known techniques such as fluorescence in situ hybridization (FISH). In an embodiment, the disclosure relates to a method of treating a CLL in a human, wherein the CLL is selected from the group consisting of IgV_{H} mutation negative CLL, ZAP-70 positive CLL, ZAP-70 methylated at CpG3 CLL, CD38 positive CLL, chronic lymphocytic leukemia characterized by a 17p13.1 (17p) deletion, and CLL characterized by a 11q22.3 (11q) deletion.

In some embodiments, the hyperproliferative disorder is a CLL wherein the CLL has undergone a Richter's transformation. Methods of assessing Richter's transformation, which is also known as Richter's syndrome, are described in Jain and O'Brien, Oncology, 2012, 26, 1146-52. Richter's transformation is a subtype of CLL that is observed in 5-10% of patients. It involves the development of aggressive lymphoma from CLL and has a generally poor prognosis.

In some embodiments, the hyperproliferative disorder is a CLL or SLL in a patient, wherein the patient is sensitive to lymphocytosis. In an embodiment, the disclosure relates to a method of treating CLL or SLL in a patient, wherein the patient exhibits lymphocytosis caused by a disorder selected from the group consisting of a viral infection, a bacterial infection, a protozoal infection, or a post-splenectomy state. In an embodiment, the viral infection in any of the foregoing embodiments is selected from the group consisting of infectious mononucleosis, hepatitis, and cytomegalovirus. In an embodiment, the bacterial infection in any of the foregoing embodiments is selected from the group consisting of pertussis, tuberculosis, and brucellosis.

In some embodiments, the hyperproliferative disorder is a blood cancer. In certain embodiments, the blood cancer is leukemia, such as acute myeloid leukemia (AML), chronic myeloid leukemia (CML), acute lymphocytic lymphoma (ALL), and chronic lymphocytic leukemia (CLL). In certain embodiments, the blood cancer is a non-Hodgkin lymphoma, such as B-cell or T-cell lymphoma. B-cell lymphomas include diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphomas, extranodal marginal zone B-cell lymphomas, nodal marginal zone B-cell lymphoma, splenic marginal zone B-cell lymphoma, Burkitt lymphoma, lymphoplasmacytic lymphoma, and primary central nervous system lymphoma. T-cell lymphomas include precursor T-lymphoblastic lymphoma, peripheral T-cell lymphomas, cutaneous T-cell lymphomas, adult T-cell lymphoma with subtypes: smoldering chronic, acute, and lymphoma, angioimmunoblastic T-cell lymphoma, extranodal natural killer/T-cell lymphoma, nasal type, enteropathy-associated intestinal T-cell lymphoma (EATL) with subtypes I and II, and anaplastic large cell lymphoma (ALCL).

Pharmaceutical compositions disclosed herein may be in the form of a liquid formulation, a solid formulation or a combination thereof. Non-limiting examples of formulations may include a tablet, a capsule, a pill, a gel, a paste, a liquid solution and a cream. In some instances, the therapeutic agent, e.g., compound or salt of Formula I, Ia, or Ib, may be in a crystallized form. In pharmaceutical compositions comprising two or more therapeutic agents, each agent may be crystallized separately and then combined or they may be crystallized together. Compositions may comprise two or more therapeutic agents in one or more physical state. For example, a composition may be a tablet comprising one therapeutic agent in a solid formulation and another therapeutic agent or drug in a gel formulation. In certain embodiments, the composition is a single pharmaceutical composition comprising a compound or salt of Formula I, Ia, or Ib in a first physical state and an additional therapeutic agent, e.g., anticancer agent in a second physical state.

The compositions of the present disclosure may further comprise an excipient or an additive. Excipients may include any and all solvents, coatings, chelating agents, flavorings, colorings, lubricants, disintegrants, preservatives, sweeteners, anti-foaming agents, buffering agents, polymers, antioxidants, binders, diluents, and vehicles (or carriers). Generally, the excipient is compatible with the therapeutic compositions of the present disclosure.

Liquid preparations for oral administration can take the form of, for example, solutions, syrups or suspensions, or they can be presented as a dry product for reconstitution with water or other suitable vehicles before use. Such liquid preparations can be prepared by conventional approaches with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

This disclosure further encompasses anhydrous compositions and dosage forms comprising an active ingredient, since water can facilitate the degradation of some compounds. Anhydrous compositions and dosage forms of the present disclosure can be prepared using anhydrous or low moisture containing ingredients and low moisture or low humidity conditions. Compositions and dosage forms of the present disclosure which contain lactose can be made anhydrous if substantial contact with moisture and/or humidity during manufacturing, packaging, and/or storage is expected. An anhydrous composition can be prepared and stored such that its anhydrous nature is maintained. Accordingly, anhydrous compositions can be packaged using materials that prevent exposure to water such that they can be included in suitable formulary kits. Examples of suitable packaging include, but are not limited to, hermetically sealed foils, plastic, unit dose containers, blister packs, and strip packs.

An ingredient described herein can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Binders suitable for use in dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

When aqueous suspensions and/or elixirs are desired for oral administration, the active ingredient therein can be combined with various sweetening or flavoring agents, coloring matter or dyes and, if so desired, emulsifying and/or suspending agents, together with such diluents as water, ethanol, propylene glycol, glycerin and various combinations thereof.

In one embodiment, the composition can include a solubilizer to ensure good solubilization and/or dissolution of the compound of the present disclosure and to minimize precipitation of the compound of the present disclosure. This can be especially important for compositions for non-oral use, e.g., compositions for injection. A solubilizer can also be added to increase the solubility of the hydrophilic drug and/or other components, such as surfactants, or to maintain the composition as a stable or homogeneous solution or dispersion.

Pharmaceutical compositions described herein may be suitable for oral administration to a subject in need thereof. In some cases, slow release formulations for oral administration may be prepared in order to achieve a controlled release of the active agent in contact with the body fluids in the gastrointestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

In some embodiments, the compositions can be formulated in a food composition. For example, the compositions can be a beverage or other liquids, solid food, semi-solid food, with or without a food carrier. For example, the compositions can include a black tea supplemented with any of the compositions described herein. The composition can be a dairy product supplemented any of the compositions described herein. In some embodiments, the compositions can be formulated in a food composition. For example, the compositions can comprise a beverage, solid food, semi-solid food, or a food carrier.

In certain embodiments, the pharmaceutical formulations can be in a form suitable for parenteral injection as a sterile suspension, solution, or emulsion in oily or aqueous vehicles, and can contain formulation agents such as suspending, stabilizing, and/or dispersing agents. Pharmaceutical formulations for parenteral administration include, for example, aqueous solutions of the active compounds in water-soluble form. Suspensions of the active compounds can be prepared, for example, as oily injection suspensions. Suitable lipophilic solvents or vehicles include fatty oils such as sesame oil, or synthetic fatty acid esters, such as ethyl oleate, isopropyl palmitate, or medium chain triglycerides, or liposomes. In preferred embodiments, a formulation for parenteral administration is an aqueous suspension.

The compound described herein may be present in a composition within a range of concentrations, the range being defined by an upper and lower value selected from any of the preceding concentrations. For example, the compound or salt of the disclosure may be present in the formulation at a concentration of from about 1 nM to about 100 mM, about 10 nM to about 10 mM, about 100 nM to about 1 mM, about 500 nM to about 1 mM, about 1 mM to about 50 mM, about 10 mM to about 40 mM, about 20 mM to about 35 mM, or about 20 mM to about 30 mM.

Methods for the preparation of compositions comprising the compounds described herein can include formulating the compounds with one or more inert, pharmaceutically-acceptable excipients. Liquid compositions include, for example, solutions in which a compound is dissolved, emulsions comprising a compound, or a solution containing liposomes, micelles, or nanoparticles comprising a compound as disclosed herein. These compositions can also contain minor amounts of nontoxic, auxiliary substances, such as wetting or emulsifying agents, pH buffering agents, and other pharmaceutically-acceptable additives.

Formulations for injection can be presented in unit dosage form, for example, in ampoules or in multi-dose containers, with an added preservative. The compositions can take such forms as suspensions, solutions or emulsions in oily or aqueous vehicles, and can contain formulatory agents such as suspending, stabilizing and/or dispersing agents. The compositions can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Pharmaceutical formulations for parenteral administration include aqueous and non-aqueous (oily) sterile injection solutions of the active compounds which can contain antioxidants, buffers, bacteriostats and solutes which render the formulation isotonic with the blood of the intended recipient; and aqueous and non-aqueous sterile suspensions which can include suspending agents and thickening agents. Suitable lipophilic solvents or vehicles include fatty oils such as sesame oil, or synthetic fatty acid esters, such as ethyl oleate or triglycerides, or liposomes.

A composition described herein, e.g., a pharmaceutical composition of a compound or salt of Formula I, Ia, or Ib, or an additional therapeutic agent, e.g., anticancer agent, or a co-formulation of a compound of Formula I, Ia, or Ib with an additional therapeutic agent, e.g., anticancer agent, can be administered once or more than once each day. The composition may be administered serially (e.g., taken every day without a break for the duration of the treatment regimen). In some cases, the treatment regime can be less than a week, a week, two weeks, three weeks, a month, or greater than a month. In some cases, a composition of the disclosure is administered over a period of at least 12 weeks. In other cases, the composition is administered for a day, at least two consecutive days, at least three consecutive days, at least four consecutive days, at least five consecutive days, at least six consecutive days, at least seven consecutive days, at least eight consecutive days, at least nine consecutive days, at least ten consecutive days, or at least greater than ten consecutive days. In some cases, a therapeutically effective amount can be administered one time per week, two times per week, three times per week, four times per week, five times per week, six times per week, seven times per week, eight times per week, nine times per week, 10 times per week, 11 times per week, 12 times per week, 13 times per week, 14 times per week, 15 times per week, 16 times per week, 17 times per week, 18 times per week, 19 times per week, 20 times per week, 25 times per week, 30 times per week, 35 times per week, 40 times per week, or greater than 40 times per week. In some cases, a therapeutically effective amount can be administered one time per day, two times per day, three times per day, four times per day, five times per day, six times per day, seven times per day, eight times per day, nine times per day, 10 times per day, or greater than 10 times per day. In some cases, the composition is administered at least twice a day. In further cases, the composition is administered at least every hour, at least every two hours, at least every three hours, at least every four hours, at least every five hours, at least every six hours, at least every seven hours, at least every eight hours, at least every nine hours, at least every 10 hours, at least every 11 hours, at least every 12 hours, at least every 13 hours, at least every 14 hours, at least every 15 hours, at least every 16 hours, at least every 17 hours, at least every 18 hours, at least every 19 hours, at least every 20 hours, at least every 21 hours, at least every 22 hours, at least every 23 hours, or at least every day.

Pharmaceutical compositions of the disclosure can be administered either acutely or chronically. Pharmaceutical compositions of the disclosure can be administered as a single treatment or as a course of treatment. Treatments can be administered once per day, twice per day, three times per day, in the morning, in the evening, before sleeping, or continuously throughout the day. Treatments can be applied every day, every other day, every three days, twice weekly, once weekly, every other week, monthly, every six weeks, every other month, every three months, every six months, annually, every other year, every 5 years, or as required.

In certain embodiments, the dose of drug being administered may be temporarily reduced or temporarily suspended for a certain length of time. In certain embodiments, the patient will have a drug holiday wherein the patient does not receive the drug or receives a reduced amount of the drug for a period of time. A drug holiday can be, for example, between 2 days and 1 year, including by way of example only, 2 days, 3 days, 4 days, 5 days, 6 days, 7 days, 10 days, 12 days, 15 days, 20 days, 28 days, or more than 28 days. A drug holiday may be for about 1 month, about 2 months, about 3 months, about 4 months, about 5 months, about 6 months, about 7 months, about 8 months, about 9 months, about 10 months, about 11 months or about 12 months. The dose reduction during a drug holiday can be, for example, by 10%-100% of the original administered dose, including by way of example only 10%, 15%, 20%, 25%, 30%, 35%, 40%, 45%, 50%, 55%, 60%, 65%, 70%, 75%, 80%, 85%, 90%, 95%, and 100%. For further examples the dose reduction can be between 10% and 100%, between 20% and 80%, between 30% and 70%, between 50% and 90%, between 80% and 100% or between 90% and 100%.

Once improvement of the patient's conditions has occurred, a maintenance dose can be administered if necessary. Subsequently, the dosage or the frequency of administration, or both, can be reduced, as a function of the symptoms, to a level at which the improved disease, disorder or condition can be retained.

Additional methods for administering the formulations described herein include, for example, limited to delivery via enteral routes including oral, gastric or duodenal feeding tube, rectal suppository, rectal enema, parenteral routes, injection, infusion, intraarterial, intracardiac, intradermal, intraduodenal, intramedullary, intramuscular, intraosseous, intraperitoneal, intrathecal, intravascular, intravenous, intravitreal, intracameral, epidural, subcutaneous, inhalational, transdermal, transmucosal, sublingual, buccal, topical, epicutaneous, dermal, enemaear drops, intranasal, and vaginal administration. The compounds described herein can be administered locally to the area in need of treatment, by for example, local infusion during surgery, topical application such as creams or ointments, injection, catheter, or implant. The administration can also be by direct injection at the site of a diseased tissue or organ.

The length of the period of administration and/or the dosing amounts can be determined by a physician or any other type of clinician. The physician or clinician can observe the subject's response to the administered compositions and adjust the dosing based on the subject's performance. For example, dosing for subjects that show reduced effects in energy regulation can be increased to achieve desired results.

In some embodiments, the combination therapies described herein can be administered together at the same time in the same route, or administered separately. In some embodiments, the components in the compositions can be administered using the same or different administration routes.

In some embodiment, the disclosure also provides for methods of manufacturing the compositions described herein. In some embodiments, the manufacture of a composition described herein comprises mixing or combining two or more components.

In some embodiments, the compositions can be combined or mixed with a pharmaceutically active or therapeutic agent, a carrier, and/or an excipient. Examples of such components are described herein. The combined compositions can be formed into a unit dosage as tablets, capsules, gel capsules, slow-release tablets, or the like.

In some embodiments, the composition is prepared such that a solid composition containing a substantially homogeneous mixture of the one or more components is achieved, such that the one or more components are dispersed evenly throughout the composition so that the composition can be readily subdivided into equally effective unit dosage forms such as tablets, pills and capsules.

A unit dose may be packaged into a container to be transferred to the user. A unit dose may be packaged in a tube, a jar, a box, a vial, a bag, a tray, a drum, a bottle, a syringe, or a can.

Another aspect of the disclosure provides for achieving desired effects in one or more subjects after administration of a combination composition described herein for a specified time period. For example, the beneficial effects of the compositions described herein can be observed after administration of the compositions to the subject for 1, 2, 3, 4, 6, 8, 10, 12, 24, or 52 weeks.

In certain embodiments, the combination therapies described herein may be administered by a combination treatment regimen. A combination treatment regimen can encompass treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. The disclosure also includes treatments in which a compound described herein, or a pharmaceutically acceptable salt thereof, and the second agent being used in combination are administered simultaneously or at different times and/or at decreasing or increasing intervals during the treatment period.

Combination treatment further includes periodic treatments that start and stop at various times to assist with the clinical management of the patient.

In certain embodiments, the combination therapy can provide a therapeutic advantage in view of the differential toxicity associated with the two treatment modalities. For example, treatment with CDK inhibitors such as those described herein can lead to a particular toxicity that is not seen with the anticancer agent, e.g., BCL-2 inhibitor or proteasome inhibitor, and vice versa. As such, this differential toxicity can permit each treatment to be administered at a dose at which said toxicities do not exist or are minimal, such that together the combination therapy provides a therapeutic dose while avoiding the toxicities of each of the constituents of the combination agents. Furthermore, when the therapeutic effects achieved as a result of the combination treatment are synergistic, the doses of each of the agents can be reduced even further, thus lowering the associated toxicities to an even greater extent.

The compounds described herein or the pharmaceutically acceptable salts thereof, as well as combination therapies, may be administered before, during or after the occurrence of a disease or condition, and the timing of administering the composition containing a compound varies. The compounds described herein can be used as a prophylactic and may be administered continuously to subjects with a propensity to develop conditions or diseases in order to prevent the occurrence of the disease or condition. The compounds described herein and compositions thereof may be administered to a subject during or as soon as possible after the onset of the symptoms. A compound described herein may be administered as soon as is practicable after the onset of a disease or condition is detected or suspected, and for a length of time necessary for the treatment of the disease.

The following clauses describe certain embodiments.
Clause 1. A method of treating a KRAS mutant cancer comprising administering to a subject in need thereof a therapeutically effective amount of a CDK inhibitor represented by Formula I:

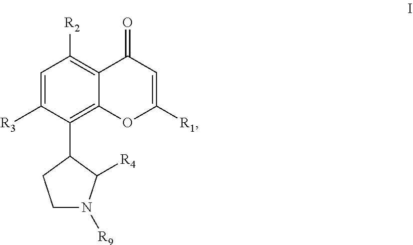

or a pharmaceutically acceptable salt thereof, wherein:
R₁ is optionally substituted phenyl;
R₂ and R₃ are each independently selected from hydroxy and —OR₈, wherein R₈ is optionally substituted C₁-C₁₀-alkyl;
R₄ is optionally substituted C₁-C₄-alkyl; and
R₉ is hydrogen or optionally substituted C₁-C₄-alkyl.
Clause 2. The method of clause 1, wherein the compound or salt of Formula I is represented by Formula Ia:

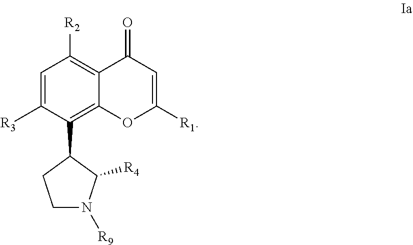

Clause 3. The method of clause 1 or clause 2, wherein R₁ is optionally substituted with one or more substituents independently selected from hydroxy, cyano, halo, amino, C₁-C₄-alkyl, C₁-C₄-alkoxy, C₁-C₄-hydroxyalkyl, C₁-C₄-haloalkyl, and nitro.
Clause 4. The method of clause 3, wherein R₁ is substituted with one or more substituents independently selected from halo and C₁-C₄-haloalkyl.
Clause 5. The method of clause 4, wherein R₁ is 2-chloro-4-trifluoromethylphenyl.
Clause 6. The method of any one of clauses 1 to 5, wherein R₂ and R₃ are each independently selected from hydroxy and —OR₈, wherein R₈ is C₁-C₁₀-alkyl optionally substituted with one or more substituents independently selected from hydroxy, cyano, halo, amino, ═O, ═S, C₁-C₄-alkoxy, and nitro.
Clause 7. The method of clause 6, wherein R₂ and R₃ are each hydroxy.
Clause 8. The method of any of clauses 1 to 7, wherein R₄ is C₁-C₄-alkyl substituted with one or more substituents selected from hydroxy, cyano, halo, amino, ═O, ═S, C₁-C₄-alkoxy, and nitro.
Clause 9. The method of any of clauses 1 to 7, wherein R₄ is —CH₂—OH.
Clause 10. The method of any of clauses 1 to 9, wherein R₉ is C₁-C₄-alkyl optionally substituted with hydroxy, cyano, halo, amino, ═O, ═S, C₁-C₄-alkoxy, and nitro.
Clause 11. The method of any of clauses 1 to 9, wherein R₉ is methyl.
Clause 12. The method of clause 1, wherein the compound of Formula I is represented by formula Ib:

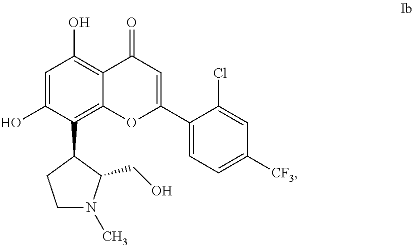 [SMILES: CN1CC[C@@H](c2c(O)cc(O)c3c(=O)cc(-c4ccc(C(F)(F)F)cc4Cl)oc23)[C@@H]1CO]

or a pharmaceutically acceptable salt thereof.
Clause 13. The method of any of clauses 1 to 12, further comprising an additional therapeutic agent.
Clause 14. The method of clause 13, wherein the therapeutic agent is an anticancer agent.
Clause 15. The method of clause 14, wherein the anticancer agent is selected from Sotorasib (AMG510), Adagrasib (MRTX849), Onvansertib, Volasertib, and ME-344.
Clause 16. The method of clause 14, wherein the anticancer agent is selected from a KRAS inhibitor disclosed herein, a TKI+RAF inhibitor disclosed herein, a RAF inhibitor disclosed herein, a RAF+MEK inhibitor disclosed herein, a MEK inhibitor disclosed herein, and an ERK inhibitor disclosed herein.
Clause 17. The method of any of clauses 1 to 16, wherein the KRAS mutant cancer is characterized by a mutation selected from G12A, G12C, G12D, G12S, G12V, G13C, G13D, and Q61H.
Clause 18. The method of any of clauses 1 to 17, wherein the cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphomas, extranodal marginal zone B-cell lymphomas, nodal marginal zone B-cell lymphoma, splenic marginal zone B-cell lymphoma, Burkitt lymphoma, lymphoplasmacytic lymphoma, and primary central nervous system lymphoma.
Clause 19. The method of any of clauses 1 to 17, wherein the cancer is selected from pancreatic cancer, lung cancer, colorectal cancer, esophageal cancer, and ovarian cancer.
Clause 20. The method of any of clauses 1 to 17, wherein the cancer is selected from NSCLC, SCLC, CRC, pancreatic, TNBC, melanoma, breast cancer, and liver cancer.
Clause 21. The method of any of clauses 1 to 20, wherein the compound of Formula I is (+)-trans-2-(2-chloro-4-trifluoromethylphenyl)-5,7-dihydroxy-8-(2-hydroxymethyl-1-methyl-pyrrolidin-3-yl)-chromen-4-one hydrochloride.
Clause 22. The method of any of clauses 1 to 20, wherein the compound of Formula I is a polymorph salt described herein.
Clause 22. The method of any of clauses 1 to 20, wherein the compound of Formula I is a polymorph salt described herein.
Clause 23. A method of treating a disease or disorder in a subject, the method comprising administering to the subject a therapeutically effective amount of voruciclib, wherein the disease or disorder is selected from chronic lymphocytic leukemia, non-Hodgkin's lymphoma, diffuse large B-cell lymphoma, mantle cell lymphoma, follicular lymphoma, B-cell lymphoproliferative disease, B cell acute lymphoblastic leukemia, Waldenström's macroglobulinemia, Burkitt's leukemia, Hodgkin's disease, multiple myeloma, acute myeloid leukemia, juvenile myelomonocytic leukemia, hairy cell leukemia, mast cell leukemia, mastocytosis, myeloproliferative disorders (MPDs), myeloproliferative neoplasms, polycythemia vera (PV), essential thrombocythemia (ET), primary myelofibrosis (PMF), myelodysplastic syndrome, chronic myelogenous leukemia (BCR-ABL1-positive), chronic neutrophilic leukemia, chronic eosinophilic leukemia, primary central nervous system (CNS) lymphoma, primary multifocal lymphoma of peripheral nervous system (PNS), thymus cancer, brain cancer, glioblastoma, lung cancer, squamous cell cancer, skin cancer (e.g., melanoma), eye cancer, retinoblastoma, intraocular melanoma, oral cavity and oropharyngeal cancers, bladder cancer, gastric cancer, stomach cancer, pancreatic cancer, breast cancer, cervical cancer, head and neck cancer, renal cancer, kidney cancer, liver cancer, ovarian cancer, prostate cancer, colorectal cancer, bone cancer (e.g., metastatic bone cancer), esophageal cancer, testicular cancer, gynecological cancer, thyroid cancer, epidermoid cancer, AIDS-related cancer (e.g., lymphoma), viral-induced cervical carcinoma (human papillomavirus), nasopharyngeal carcinoma (Epstein-Barr virus), Kaposi's sarcoma, primary effusion lymphoma (Kaposi's sarcoma herpesvirus), hepatocellular carcinoma (hepatitis B and hepatitis C viruses), T-cell leukemias (Human T-cell leukemia virus-1), benign hyperplasia of the skin, restenosis, benign prostatic hypertrophy, tumor angiogenesis, chronic inflammatory disease, rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, ulcerative colitis, atopic dermatitis, pouchitis, spondylarthritis, uveitis, Behcet's disease, polymyalgia rheumatica, giant-cell arteritis, sarcoidosis, Kawasaki disease, juvenile idiopathic arthritis, hidratenitis suppurativa, Sjögren's syndrome, psoriatic arthritis, juvenile rheumatoid arthritis, ankylosing spondylitis, Crohn's disease, lupus, and lupus nephritis.
Clause 24. A method of treating a hyperproliferative disease or disorder in a subject, the method comprising administering to the subject a therapeutically effective amount of voruciclib, wherein the hyperproliferative disease or disorder is selected from acute lymphoblastic leukemia, acute myeloid leukemia, chronic lymphocytic leukemia, non-Hodgkin's lymphoma, diffuse large B-cell lymphoma, mantle cell lymphoma, follicular lymphoma, B-cell lymphoproliferative disease, B cell acute lymphoblastic leukemia, and Waldenström's macroglobulinemia.
Clause 25. A method of treating a blood cancer in a subject, the method comprising administering to the subject a therapeutically effective amount of voruciclib.
Clause 26. The method of clause 25, wherein the blood cancer is selected from acute myeloid leukemia (AML), chronic myeloid leukemia (CML), acute lymphocytic lymphoma (ALL), and chronic lymphocytic leukemia (CLL).
Clause 27. A method of treating a hyperproliferative disease or disorder in a subject, the method comprising administering to the subject a therapeutically effective amount of voruciclib, wherein the hyperproliferative disease or disorder is a KRAS mutant cancer.
Clause 28. The method of clause 27, wherein the KRAS mutant cancer is characterized by a mutation selected from G12A, G12C, G12D, G12S, G12V, G13C, G13D, and Q61H.
Clause 29. The method of clause 27 or 28, wherein the cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphomas, extranodal marginal zone B-cell lymphomas, nodal marginal zone B-cell lymphoma, splenic marginal zone B-cell lymphoma, Burkitt lymphoma, lymphoplasmacytic lymphoma, and primary central nervous system lymphoma.
Clause 30. The method of any one of clauses 27 to 29, wherein the cancer is selected from pancreatic cancer, lung cancer, colorectal cancer, esophageal cancer, and ovarian cancer.
Clause 31. The method of any one of clauses 27 to 29, wherein the cancer is selected from NSCLC, SCLC, CRC, pancreatic, TNBC, melanoma, breast cancer, and liver cancer.
Clause 32. The method of any one of clauses 23 to 31, wherein the disease or disorder is a relapsed/refractory (R/R) disease or disorder.
Clause 33. The method of any one of clauses 23 to 32, wherein voruciclib comprises a voruciclib salt comprising a counterion corresponding to an acid selected from 1,5-naphthalenedisulfonic acid, 1-hydroxy-2-naphthoic acid, benzenesulfonic acid, benzoic acid, dibenzoyl-L-tartaric acid, ethanesulfonic acid, gentisic acid, hydrobromic acid, hydrochloric acid, maleic acid, malonic acid, oxalic acid, ortho-phosphoric acid, sulfuric acid, and p-toluenesulfonic acid.
Clause 34. The method of any one of clauses 23 to 32, wherein voruciclib comprises a crystal form of voruciclib, comprising voruciclib free base or a voruciclib salt comprising a counterion corresponding to an acid selected from 1,5-naphthalenedisulfonic acid, 1-hydroxy-2-naphthoic acid, benzenesulfonic acid, benzoic acid, dibenzoyl-L-tartaric acid, ethanesulfonic acid, gentisic acid, hydrobromic acid, hydrochloric acid, maleic acid, malonic acid, oxalic acid, ortho-phosphoric acid, sulfuric acid, and p-toluenesulfonic acid.
Clause 35. The method of any one of clauses 23 to 32, wherein voruciclib comprises a crystal form of voruciclib characterized by an X-ray powder diffraction pattern comprising one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38°±0.2°, 24.10°±0.2°, 24.980°±0.20, 25.94°±0.20, 27.26°±0.20, 28.50°±0.20, and 32.820°±0.2° 2θ.
Clause 36. The method of clause 35, wherein the crystal form comprises voruciclib malonate.
Clause 37. The method of any one of clauses 23 to 32, wherein voruciclib comprises a crystal form of voruciclib characterized by an X-ray powder diffraction pattern comprising one or more peaks selected from 5.06°±0.2°, 6.42°±0.2°, 9.34°±0.2°, 10.14°±0.2°, 12.30°±0.2°, 13.66°±0.2°, 14.14°±0.2°, 15.82°±0.2°, 17.02°±0.2°, 19.74°±0.2°, 20.38°±0.2°, 21.82°±0.20, 22.66°±0.20, 24.62°±0.20, 25.78°±0.20, 26.58°±0.20, 28.66°±0.20, and 29.98°±0.2° 2θ.
Clause 38. The method of clause 37, wherein the crystal form comprises voruciclib dibenzoyl-tartrate.
Clause 39. The method of any one of clauses 23 to 32, wherein voruciclib comprises a crystal form of voruciclib characterized by an X-ray powder diffraction pattern comprising one or more peaks selected from 4.94°±0.2°, 6.78°±0.2°, 9.34°±0.2°, 10.94°±0.2°, 12.70°±0.2°, 13.38°±0.2°, 14.90°±0.2°, 15.66°±0.2°, 17.54°±0.2°, 18.82°±0.2°, 22.02°±0.2°, 23.980°±0.20, 24.780°±0.20, 25.300°±0.20, 26.660°±0.20, and 29.980°±0.2° 2θ.
Clause 40. The method of clause 39, wherein the crystal form comprises voruciclib phosphate.
Clause 41. The method of any one of clauses 23 to 32, wherein voruciclib comprises a crystal form of voruciclib characterized by an X-ray powder diffraction pattern comprising one or more peaks selected from 6.86°±0.2°, 12.66°±0.2°, 13.58°±0.2°, 14.74°±0.2°, 15.98°±0.2°, 19.38°±0.2°, 23.94°±0.2°, 24.78°±0.2°, and 25.94°±0.2° 2θ.
Clause 42. The method of clause 41, wherein the crystal form comprises voruciclib oxalate.
Clause 43. The method of any one of clauses 23 to 32, wherein voruciclib comprises a crystal form of voruciclib characterized by an X-ray powder diffraction pattern comprising one or more peaks selected from 9.02°±0.2°, 10.50°±0.2°, 11.06°±0.2°, 12.30°±0.2°, 12.82°±0.20, 13.900°±0.20, 14.820°±0.20, 15.300°±0.20, 15.940°±0.20, 17.260°±0.20, 19.340°±0.20, 20.62°±0.2°, 22.18°±0.2, 22.86°±0.2°, 24.58°±0.2°, 25.420°±0.20, 25.86 6°±0.2°, 27.38°±0.2°, and 28.66°±0.2° 2θ.
Clause 44. The method of clause 43, wherein the crystal form comprises voruciclib napadisylate.
Clause 45. The method of any one of clauses 34 to 44, wherein the crystal form is a crystalline anhydrate.
Clause 46. The method of any one of clauses 34 to 44, wherein the crystal form is a crystalline hydrate.
Clause 47. The method of any one of clauses 23 to 32, wherein voruciclib comprises a crystal form of voruciclib malonate characterized by an X-ray powder diffraction pattern comprising one or more peaks selected from 6.36°±0.2° 2θ, 13.88°±0.2° 2θ, 7.31°±0.2° 2θ, 9.34°±0.2° 2θ, 10.05°±0.2° 2θ, 13.59°±0.2° 2θ, 14.08°±0.2° 2θ, 15.21°±0.2° 2θ, 15.67°±0.2° 2θ, 17.53°±0.2° 2θ, 18.70°±0.2° 2θ, 18.98°±0.2° 2θ, 19.38°±0.2° 2θ, 19.67°±0.2° 20, 20.16°±0.2° 2θ, 20.39°±0.2° 2θ, 21.01°±0.2° 2θ, 22.27°±0.2° 2θ, 23.35°±0.2° 2θ, 24.15°±0.2° 2θ, 24.67°±0.2° 2θ, 25.00°±0.2° 2θ, 25.18°±0.2° 2θ, 25.57°±0.2° 2θ, 25.93°±0.2° 2θ, 26.21°±0.2° 2θ, 27.19°±0.2° 2θ, and 27.38°±0.2° 2θ.
Clause 48. The method of any one of clauses 23 to 32, wherein voruciclib comprises a crystal form of voruciclib oxalate characterized by an X-ray powder diffraction pattern comprising one or more peaks selected from 6.86°±0.2° 2θ, 9.70°±0.2° 2θ, 10.84°±0.2° 2θ, 12.50°±0.2° 2θ, 12.66°±0.2° 2θ, 12.81°±0.2° 2θ, 13.41°±0.2° 2θ, 13.71°±0.2° 2θ, 14.54°±0.2° 2θ, 15.35°±0.2° 2θ, 15.83°±0.2° 2θ, 18.70°±0.2° 2θ, 19.00°±0.2° 2θ, 19.43°±0.2° 20, 19.62°±0.2° 2θ, 21.75°±0.2° 2θ, 22.75°±0.2° 2θ, 23.35°±0.2° 2θ, 23.47°±0.2° 2θ, 23.81°±0.2° 2θ, 23.98°±0.2° 2θ, 24.36°±0.2° 2θ, 24.60°±0.2° 2θ, 24.86°±0.2° 2θ, 25.11°±0.2° 2θ, 25.60°±0.2° 2θ, 25.75°±0.2° 2θ, and 26.25°±0.2° 2θ.
Clause 49. The method of any one of clauses 23 to 32, wherein voruciclib comprises a crystal form of voruciclib phosphate characterized by an X-ray powder diffraction pattern comprising one or more peaks selected from 4.930°±0.2° 2θ, 6.79°±0.2° 2θ, 9.350°±0.2° 2θ, 10.580 0.2° 2θ, 10.910 0.2° 2θ, 12.640°±0.2° 2θ, 13.350°±0.2° 2θ, 13.580°±0.2° 2θ, 14.810±0.2° 2θ, 15.600°±0.2° 2θ, 17.180 0.2° 2θ, 17.520°±0.2° 2θ, 18.320°±0.2° 2θ, 18.780°±0.20 20, 19.34°±0.2° 2θ, 19.64°±0.2° 2θ, 19.78°±0.2° 2θ, 22.02°±0.2° 2θ, 23.20°±0.2° 2θ, 23.670°±0.2° 2θ, 24.000°±0.2° 2θ, 24.710°±0.2° 2θ, 25.210°±0.2° 2θ, 25.390°±0.2° 2θ, 26.550±0.2° 2θ, 27.220°±0.2° 2θ, 28.070°±0.2° 2θ, and 29.900°±0.2° 2θ.
Clause 50. The method of any one of clauses 23 to 49, wherein voruciclib is administered at a daily dose between about 50 mg and about 100 mg, between about 100 mg and about 150 mg, between about 150 mg and about 200 mg, between about 200 mg and about 250 mg, between about 250 mg and about 300 mg, between about 300 mg and about 350 mg, between about 350 mg and about 400 mg, between about 400 mg and about 450 mg, between about 450 mg and about 500 mg, between about 500 mg and about 550 mg, between about 550 mg and about 600 mg, between about 600 mg and about 650 mg, between about 650 mg and about 700 mg, between about 700 mg and about 750 mg, between about 750 mg and about 800 mg, between about 800 mg and about 850 mg, between about 850 mg and about 900 mg, between about 900 mg and about 950 mg, or between about 950 mg and about 1,000 mg. In some embodiments, the daily dose refers to voruciclib free base. In some embodiments, the daily dose refers to a voruciclib free base amount administered as the equivalent amount of voruciclib salt or salt polymorph.
Clause 51. The method of any one of clauses 23 to 49, wherein voruciclib is administered at a daily dose of about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1,000 mg. In some embodiments, the daily dose refers to voruciclib free base. In some embodiments, the daily dose refers to a voruciclib free base amount administered as the equivalent amount of voruciclib salt or salt polymorph.
Clause 52. The method of any one of clauses 23 to 51, wherein voruciclib is administered daily for about one day, about two days, about three days, about 4 days, about 5 days, about 6 days, about 7 days, about 8 days, about 9 days, about 10 days, about 11 days, about 12 days, about 13 days, or about 14 days.
Clause 53. The method of any one of clauses 23 to 51, wherein voruciclib is administered every other day for about one day, about two days, about three days, about 4 days, about 5 days, about 6 days, about 7 days, about 8 days, about 9 days, about 10 days, about 11 days, about 12 days, about 13 days, or about 14 days.
Clause 54. The method of any one of clauses 23 to 51, wherein voruciclib is administered daily for about one week, about two weeks, about three weeks, or about 4 weeks.
Clause 55. The method of any one of clauses 23 to 51, wherein voruciclib is administered every other day for about one week, about two weeks, about three weeks, or about 4 weeks.
Clause 56. The method of any one of clauses 23 to 55, wherein voruciclib administration is paused for about one day, about two days, about three days, about 4 days, about 5 days, about 6 days, about 7 days, about 8 days, about 9 days, about 10 days, about 11 days, about 12 days, about 13 days, or about 14 days.
Clause 57. The method of any one of clauses 23 to 55, wherein voruciclib administration is paused for about one week, about two weeks, about three weeks, or about 4 weeks.
Clause 58. The method of any one of clauses 23 to 57, wherein voruciclib is administered on a 14 days on/14 days off schedule.
Clause 59. The method of any one of clauses 23 to 57, wherein voruciclib is administered on a 7 days on/14 days off schedule.
Clause 60. The method of any one of clauses 23 to 57, wherein voruciclib is administered on a 14 days on/7 days off schedule.
Clause 61. The method of any one of clauses 23 to 57, wherein voruciclib is administered on a 7 days on/7 days off schedule.
Clause 62. The method of any one of clauses 23 to 61, wherein voruciclib is administered for about one month, about two months, about three months, about 4 months, about 5 months, about 6 months, about 7 months, about 8 months, about 9 months, about 10 months, about 11 months, or about 12 months.
Clause 63. The method of any one of clauses 23 to 62, wherein voruciclib is administered in combination with an additional therapeutic agent.
Clause 64. The method of clause 63, wherein the therapeutic agent is an anticancer agent.
Clause 65. The method of clause 64, wherein the anticancer agent is selected from AMG510, MRTX849, Onvansertib, Volasertib, and ME-344.
Clause 66. The method of clause 64, wherein the anticancer agent is selected from a KRAS inhibitor, a TKI+RAF inhibitor, a RAF inhibitor, a RAF+MEK inhibitor, a MEK inhibitor, and an ERK inhibitor.
Clause 67. The method of clause 64, wherein the anticancer agent is a BCL-2 inhibitor selected from navitoclax, venetoclax, A-1155463, A-1331852, ABT-737, obatoclax, TW-37, A-1210477, AT101, HA14-1, BAM7, S44563, sabutoclax, UMI-77, gambogic acid, maritoclax, MIM1, methylprednisolone, iMAC2, Bax inhibitor peptide V5, Bax inhibitor peptide P5, Bax channel blocker, and ARRY 520 trifluoroacetate.
Clause 68. The method of clause 64, wherein the anticancer agent is a proteasome inhibitor selected from bortezomib, marizomib, ixazomib, disulfiram, epigallocatechin-3-gallate, salinosporamide A, carfilzomib, ONX 0912, CEP-18770, MLN9708, epoxomicin, MG132 and a pharmaceutically acceptable salt of any one thereof.
Clause 69. The method of any one of clauses 36 to 41, wherein the additional therapeutic agent is administered daily for about one day, about two days, about three days, about 4 days, about 5 days, about 6 days, about 7 days, about 8 days, about 9 days, about 10 days, about 11 days, about 12 days, about 13 days, or about 14 days.
Clause 70. The method of any one of clauses 63 to 69, wherein the additional therapeutic agent is administered daily for about one week, about two weeks, about three weeks, or about 4 weeks.
Clause 71. The method of any one of clauses 63 to 69, wherein the additional therapeutic agent is administered every other day for about one week, about two weeks, about three weeks, or about 4 weeks.
Clause 72. The method of any one of clauses 63 to 71, wherein the additional therapeutic agent administration is paused for about one day, about two days, about three days, about 4 days, about 5 days, about 6 days, about 7 days, about 8 days, about 9 days, about 10 days, about 11 days, about 12 days, about 13 days, or about 14 days.
Clause 73. The method of any one of clauses 63 to 71, wherein the additional therapeutic agent administration is paused for about one week, about two weeks, about three weeks, or about 4 weeks.
Clause 74. The method of any one of clauses 63 to 73, wherein the additional therapeutic agent is administered on a 14 days on/14 days off schedule.
Clause 75. The method of any one of clauses 63 to 73, wherein the additional therapeutic agent is administered on a 7 days on/7 days off schedule.
Clause 76. The method of any one of clauses 63 to 73, wherein the additional therapeutic agent is administered on a 7 days on/14 days off schedule.
Clause 77. The method of any one of clauses 63 to 73, wherein the additional therapeutic agent is administered on a 14 days on/7 days off schedule.
Clause 78. The method of any one of clauses 63 to 77, wherein the additional therapeutic agent is administered for about one month, about two months, about three months, about 4 months, about 5 months, about 6 months, about 7 months, about 8 months, about 9 months, about 10 months, about 11 months, or about 12 months.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

For all in vitro drug mechanism-of-action studies, the materials and methods outlined below were used.

### Example 1: Voruciclib, a CDK9 Inhibitor, Downregulates MYC and Inhibits Proliferation of KRAS Mutant Cancers in Preclinical Models

Mutations in KRAS at G12, G13, and Q61 are oncogenic drivers in many cancers, including lung, colorectal, pancreatic, multiple myeloma, and uterine carcinomas. KRAS mutations are frequently accompanied by stabilization of the MYC oncoprotein through increased MYC transcription and decreased protein degradation that is mediated by phosphorylation of MYC on Ser 62 by ERK and CDK9 kinases.

Voruciclib is an oral inhibitor of CDKs 9, 4, 6, and 1 currently being tested in Phase 1B clinical trials for B-cell malignancies and acute myeloid leukemia. Voruciclib inhibition of CDK9 leads to decreased expression of transcriptional targets of RNA Pol II, such as Mcl1 and MYC. To test whether Voruciclib could be effective in cancers driven by dysregulated KRAS-MYC signaling, ˜20 cancer cell lines with KRAS mutations (G12A, G12C, G12D, G12S, G12V, G13C, G13D, Q61H) were treated in preclinical studies with Voruciclib. MTS and Cell Titer Glo assays were used to monitor growth in vitro. Voruciclib decreased viability in all cell lines tested. To investigate MYC protein stability, MIA PACA-2 (G12C) cells were treated with 4 μM Voruciclib for 5-240 min, followed by SDS-PAGE and Western Blotting analysis with α-MYC and α-pSer62-MYC antibodies. Voruciclib treatment resulted in a reduction in phosphorylation of MYC on Ser 62. A 60% decrease in pSer62 was observed after 5 min that reached 80% by 60 min. In contrast, there was no decrease in total MYC protein at either 5 or 15 min. A 10% reduction in total MYC was observed at 60 min that reached 50% at 240 min. The ability of Voruciclib to inhibit tumor growth in vivo was also tested in murine xenograft models. KRAS mutant human cancer cells HCT-116 (CRC, KRAS G13D), SW-480 (CRC, KRAS G12V), and H-460 (NSCLC, KRAS Q61H) were injected subcutaneously into SCID mice. Once tumors reached 5-10 mm in diameter, Voruciclib or vehicle were administered orally at 50, 100, or 200 mg/kg OD for 11-14 days. Tumors were measured every 2-3 days, and growth inhibition relative to control was calculated. Significant tumor growth inhibition (>50%) was observed at all doses of Voruciclib tested.

Collectively, these data demonstrate that Voruciclib inhibition of CDK9 leads to reduced phosphorylation of MYC on Ser 62 followed by a decrease in total MYC protein in MIA PACA-2 cells and inhibition of growth in multiple KRAS mutant cancer cell lines in vivo and in vitro. Thus, Voruciclib is a therapeutic for cancers driven by KRAS-MYC.

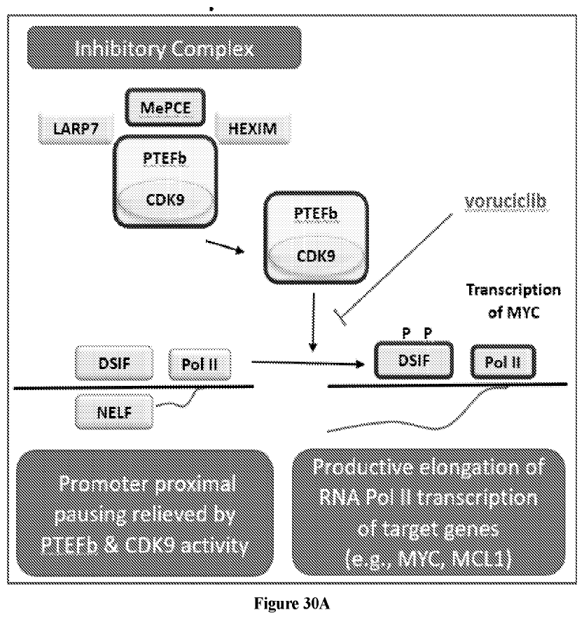

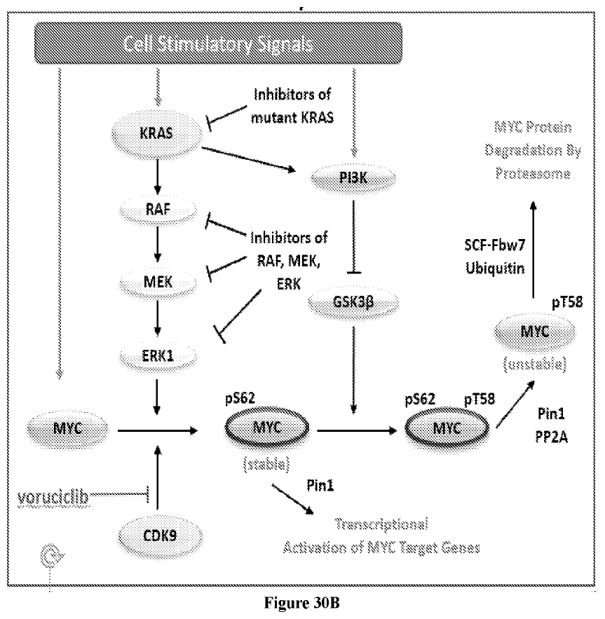

FIG. 30—CDK9 regulates transcription of MYC by RNA Pol II and MYC protein stability: Schematic illustrating (FIG. 30A) P-TEFb regulation of RNA Pol II driven transcription of MYC and (FIG. 30B) KRAS-ERK1 signaling pathway and regulation of MYC protein stability by phosphorylation of Ser 62. Proteins with decreased phosphorylation after voruciclib treatment are circled in purple. Points of CDK9 inhibition by voruciclib are noted.

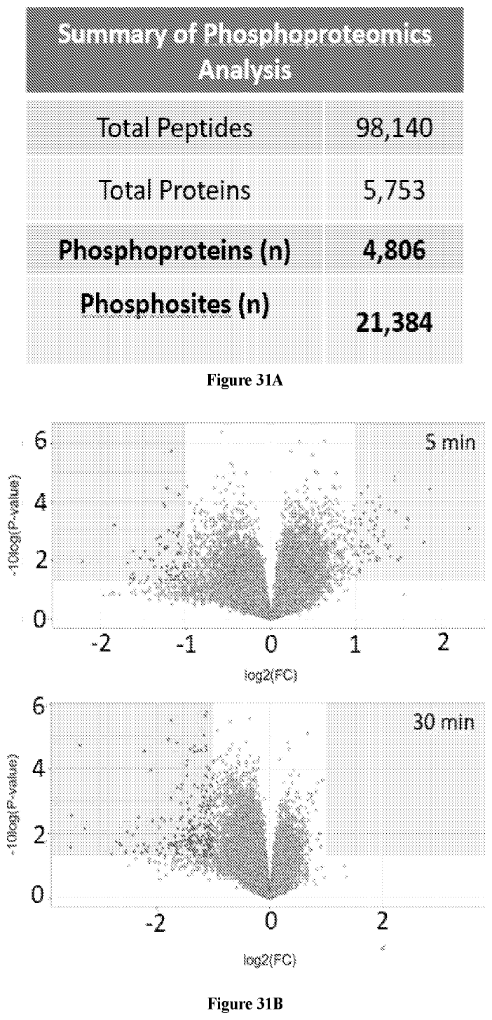

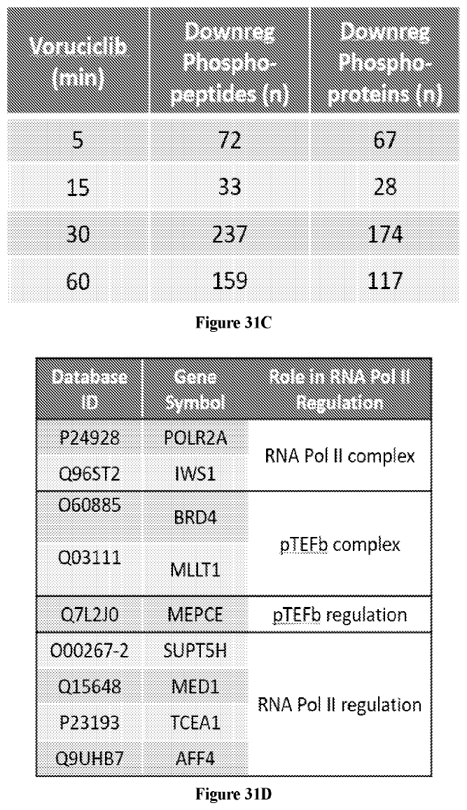

FIG. 31—Voruciclib induces rapid down regulation of RNA Pol II associated proteins that control transcription of MYC: Landscape of the voruciclib-sensitive phosphoproteome in MIA Paca-2 cells reveals rapid downregulation of phosphoproteins controlling transcription of MYC. Cells were treated with voruciclib (4 μM) over time. (FIG. 31A) Summary of peptide quantification after TMT labelling and analysis by LC-MS/MS. (FIG. 31B) Volcano plots of phosphosites (log 2 fold change vs −log 10 p-value). Significantly downregulated are in red. Significantly upregulated are in green (p<0.05. Fold change >2.0). (FIG. 31C) Summary of significantly down-regulated phosphoproteins and phosphopeptides over time. (FIG. 31D) Downregulated phosphoproteins with a role in regulation of RNA Pol II activity.

FIG. 32—Voruciclib causes rapid inhibition MYC pSer62 phosphorylation and reduces MYC protein levels. Immunoblot analyses of c-MYC, phospho-c-MYC (Ser62), and actin in MIA Paca-2 KRAS G12C mutant PDAC cells. (FIG. 32A) Cells were treated with vehicle control, voruciclib (VOR, 4 μM), or the AZD4573 (AZD, a CDK9 inhibitor, 400 nM) for the indicated times. (FIG. 32B) Cells were treated with various concentrations of voruciclib or AZD4573 for the indicated time. Relative densitometry values are indicated.

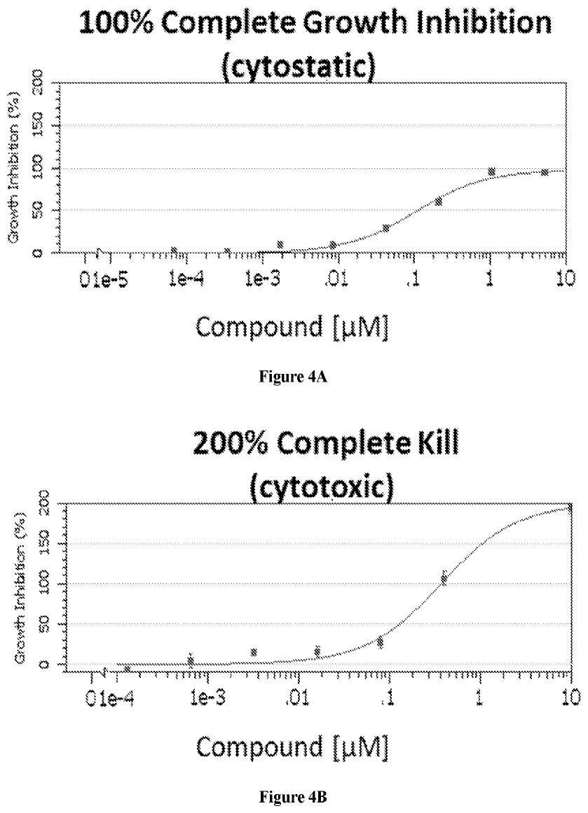

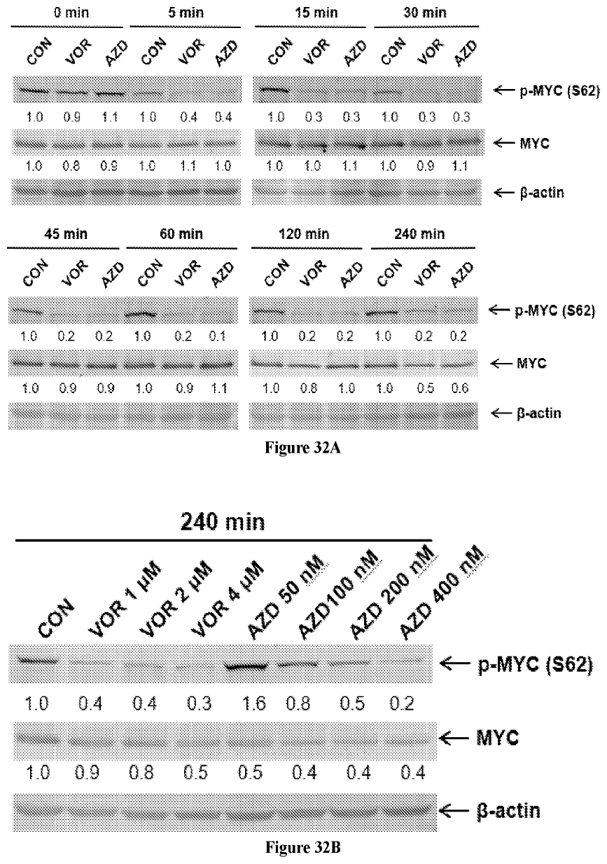

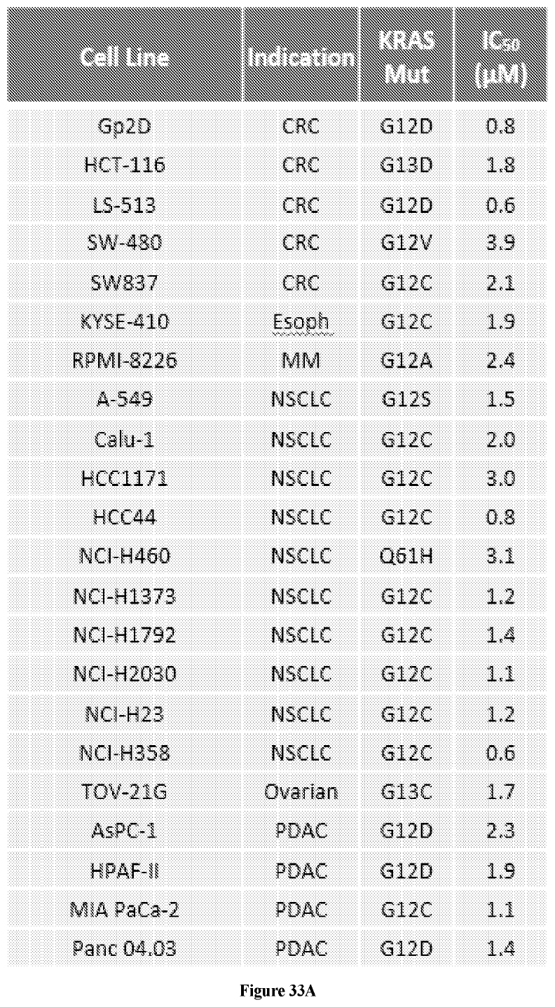

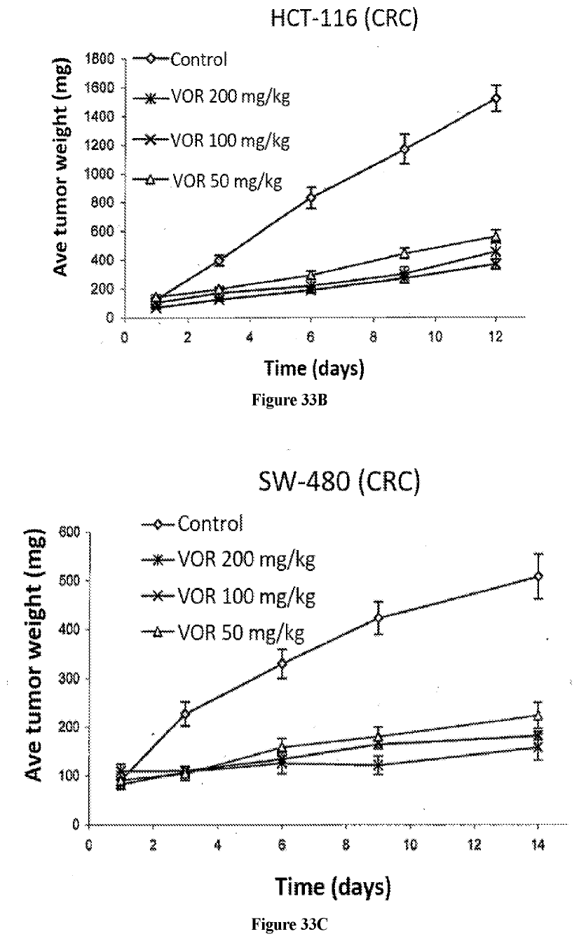

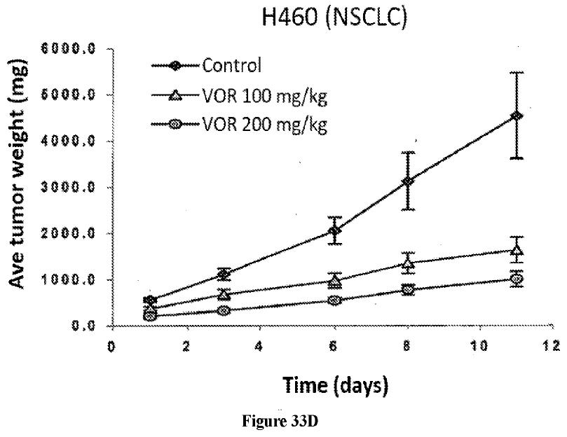

FIG. 33—Voruciclib inhibits proliferation of KRAS mutant cancer cell lines in vitro and in vivo: (FIG. 33A) Voruciclib IC50 values across multiple cell lines with KRAS mutations. Murine xenograft experiment showing tumor growth over time in mice bearing HCT-116 (G13D) (FIG. 33B), SW-480 (G12V) (FIG. 4C) or H-460 (Q61H) (FIG. 33D) tumors after treatment with voruciclib (VOR) at various doses (p.o.) for 11-14 days.

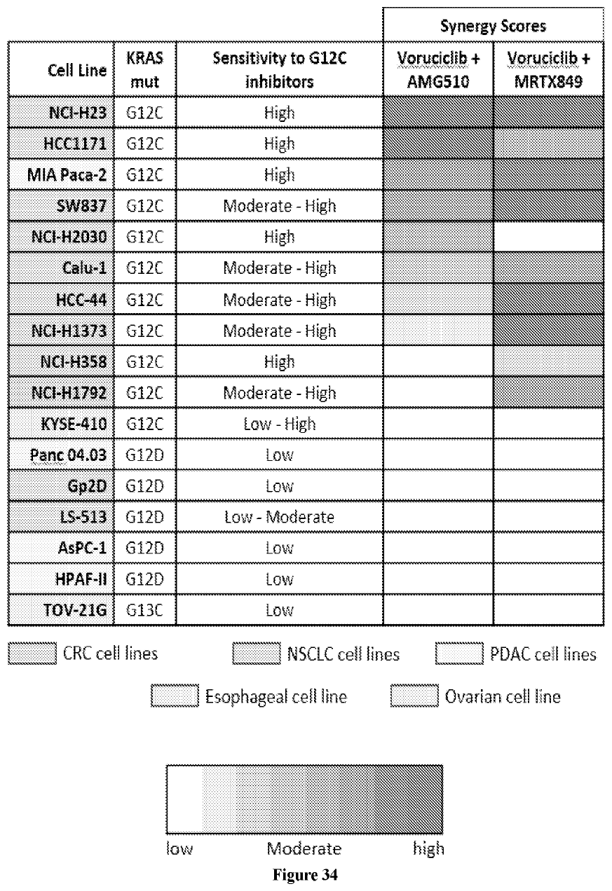

FIG. 34—Voruciclib synergizes with KRAS G12C inhibitors in vitro: Heatmap of combination activity of voruciclib with KRAS G12C inhibitors in cancer cell lines after 72 hours. Cell lines are ranked by synergy score of voruciclib in combination with either AMG510 or MTRX849. HSA, Bliss, and Loewe analyses were performed to generate the synergy scores using Chalice Analyzer. High synergy scores are represented as dark green. Moderate synergy scores are represented in shades of green. Low to moderate synergy scores are represented in white. Cell sensitivity to KRAS G12C inhibitors are ranked by EC50 scores. High (<0.1 μM), Moderate (>0.1 μM), low (>1 μM). Where sensitivities to the two inhibitors differ, a range of responses is given.

In KRAS G12C mut MIA Paca-2 pancreatic cancer cells, voruciclib treatment resulted in a rapid decrease in both phosphorylation of proteins that regulate transcription of MYC and in phosphorylation of MYC protein on Ser62, which was followed by a reduction in total MYC protein.

Voruciclib demonstrated single agent efficacy against KRAS mutant cancer cells in preclinical models and acted synergistically with KRAS G12C inhibitors in killing KRAS mutant cancer cell lines.

### Example 2: Defining the Role of CDK9 in Mutant KRAS-Driven Cancers

KRAS mutations at G12, G13, and Q61 are detected in many cancers, including: pancreatic ductal adenocarcinoma (98%), colorectal adenocarcinoma (52%), multiple myeloma (43%), lung adenocarcinoma (32%), and uterine carcinomas (25%). Stabilization of the MYC oncoprotein plays an important role in the oncogenesis driven by KRAS mutants. This stabilization is accomplished both by increasing MYC transcription and by influencing MYC protein degradation. Phosphorylation of MYC on Ser62 by ERK kinases downstream of KRAS is known to promote MYC protein stability. Recently, high throughput screens were performed to identify inhibitors of KRAS-mediated MYC protein stabilization in pancreatic and colon cancer cell lines, respectively. Inhibitors of CDK9 that promoted degradation of MYC in a KRAS-independent fashion were identified, although none of the inhibitors were specific for CDK9. Moreover, it has been showed that MYC is a direct protein substrate of CDK9, suggesting that therapeutic intervention by CDK9i could be a viable option in any cancer with deregulated MYC.

Voruciclib (VOR) inhibits CDK9 and can reduce MYC transcription by inhibiting CDK-mediated phosphorylation of Pol II. In addition, it has been demonstrated that treatment of AML cells with VOR for 3 hr leads to a decrease in MYC protein.

A variety of pancreatic, lung, and colon cancer cell lines with KRAS mutations (KRAS G12C and others) are cultured and treated with VOR for a time course (10, 20, 30, 60, 180 min), followed by lysis and analysis by SDS-PAGE and Western blot. The MIA Paca2 cells are treated with 1-4 μM of VOR, and a dose response curve can be performed in order to determine the optimal VOR conc to use with these cells. AZD4573, a CDK9i, can be used as a positive control. Cell lysates are blotted with antibodies to: MYC, pMYC-Ser62, and actin. These experiments are establishing whether VOR treatment leads to an inhibition of MYC phosphorylation and determining the optimal time point for seeing maximal reduction of phosphorylation without loss of MYC protein.

The efficacy and synergy of VOR with KRAS G12C specific inhibitors from Amgen (AMG510) and Mirati Therapeutics (MRTX849) is evaluated. A panel of cancer cell lines (lung, pancreas, ovary, esophagus, colon) harboring KRAS G12C mutations is screened with VOR in combination with AMG510 and MRTX849 at various concentrations and measure viability at 72 hr as the readout. Data is analyzed for synergy.

To define the CDK9-dependent phosphoproteome, cells are treated for a short time period with VOR followed by harvesting of the cells, IMAC enrichment, 16-plex isobaric tagging, and analysis by LC/MS/MS. A time course or different doses or drug combinations for the conditions can be performed. This experiment provides insight into novel CDK9 substrates and inroads into possible therapeutic areas for CDK9 inhibition by VOR.

### Example 3: Combining Voruciclib with PLK-1 Inhibitors in Synergy Screen

PLK1 is overexpressed in certain cancers: NSCLC, colorectal, pancreatic, melanoma, breast, ovarian, head & neck, NHL, AML, prostate, and liver. In some embodiments, PLK1 overexpression is associated with worse prognosis and lower OS in cancers: NSCLC, prostate, head and neck, melanoma. PLK1 is required for viability of cells with activating Ras mutations or inactivating p53 mutations. KRAS mutant cell lines have increased MYC and are sensitive to agents that degrade MYC (incl CDK9 inhibitors).

| Cancer | Primary RAS | |
|---|---|---|
| Tissue | Mutation | % Cancers with RAS mutation |
| Pancreatic | KRAS G12D | 90 |
| Colorectal | KRAS G12D | 30-50 |
| Lung | KRAS G12C | 19 |
| Melanoma | NRAS Q61R, | 18 |
| | Q61K | |

| PLK1 Inhibitor | IC₅₀ (nM) |
|---|---|
| Volasertib | PLK1 = 0.87 (6 & 65 fold selectivity over PLK2/3) |
| Onvansertib | PLK1 = 2 (5,000 fold selectivity) |
| GSK461364 | PLK1 = 2.2 (1,000 fold selectivity) |

Combination Synergy Screen: Voruciclib + PLK1 Inhibitor

| Cancer | | |
|---|---|---|
| Type | Cell Line | KRAS mutation |
| Colorectal | Gp2D | G12D |
| | LS513 | G12D |
| | CL40 | G12D |
| | SW837 | G12C |
| Pancreatic | AsPC-1 | G12D |
| | HPAF-11 | G12D |
| | Panc04.03 | G12D |

### Example 4: Combination Profiling of MEI-522 (Voruciclib) in 20 Cancer Cell Lines

The aim of this study was to determine the combinatorial activity of MEI Pharma's lead compound MEI-522 (Voruciclib) with several partner molecules across a panel of 20 cancer cell lines utilizing Horizon Discovery's High Throughput Screening platform. Growth inhibition was determined using a 72-hour CellTiter-Glo®2.0 proliferation assay. Combination format was a 9×9 dose matrix. Six of the 20 cell lines received the original two combinations whilst the remaining 14 cell lines received two additional combinations resulting in four combinations in total. Results have been supplied as both Chalice Analyzer files (.mcc) and raw luminescence data.

Voruciclib Single Agent Activity

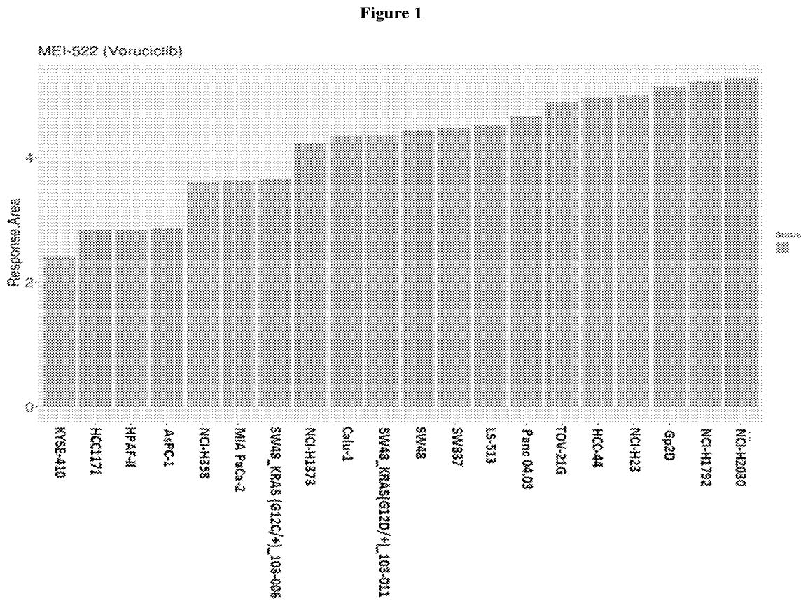

FIG. 1. Cell line response to MEI-522 (Voruciclib) at 72 hours ranked by Response Area. Single agent activity of MEI-522 (Voruciclib) was extracted from the combination dose matrices. Cell line responses are ranked by median response.

Single Agent Activity Stratified by KRAS Status

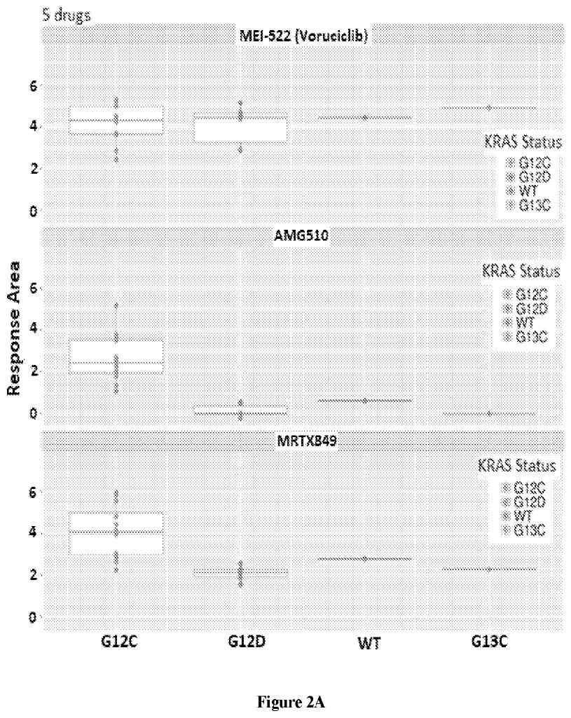

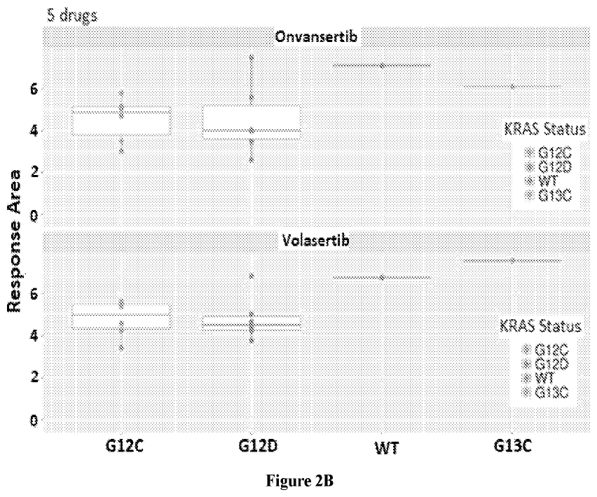

FIGS. 2A-2B. Single agent response stratified by KRAS status at 72 hours. KRAS status ranked by median Response Area for MEI-522 (Voruciclib). Single agent activity of MEI-522 (Voruciclib) was extracted from the growth inhibition combination dose matrices.

MEI-522 (Voruciclib) Combination Activity

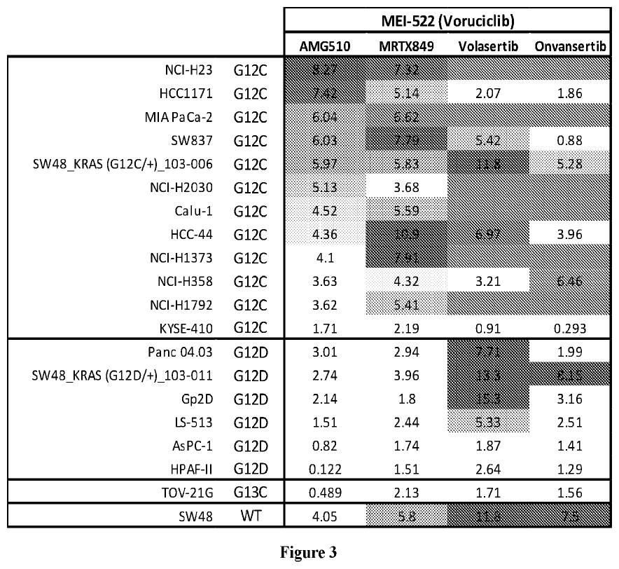

FIG. 3. Heatmap of combination activity of MEI-522 (Voruciclib) with various enhancers in the cell line panel after 72 hours. Combination activity is represented by Synergy Score as determined by the Chalice Analyzer. Chalice Analyzer provides a heatmap in matrix view comparing strength of the Synergy scores between the cell lines. Cell lines are ranked by synergy score of MEI-522 (Voruciclib) in combination with AMG510. Synergy scores (>7.71) are represented as red. Moderate synergy scores (between 3.92 and 7.71) are represented in shades of red. Low to moderate synergy scores (<3.92) are represented in white. Where a combination has not been assessed, this is represented by a grey box.

### Single Agent Responses

TABLE 1
Single agent data metrics across the cell line panel. Potency and efficacy metrics from a screen
of twenty cell lines were derived from logistic curves fitted to growth inhibition data using
Horizon's Chalice software. Single agent response metrics were extracted from the dose matrices.
EC₅₀ and GI₅₀ reported to 4 decimal places whilst Response Are (AUC) is reported to 2 decimal
places. Data metrics were determined by chalice and reported where determined.

| | | | EC₅₀ | GI₅₀ | Max Response | Response | |
|---|---|---|---|---|---|---|---|
| Cell Line | Tissue | Compound | (μM) | (μM) | (% GI) | Area | N |
| MEI-522 (Voruciclib) | | | | | | | |
| AsPC-1 | Pancreas | MEI-522(Voruciclib) | 1.3520 | 1.3406 | 99 | 2.86 | 12 |
| Calu-1 | Lung | MEI-522(Voruciclib) | 2.0747 | 1.6772 | 159 | 4.35 | 6 |
| Gp2D | Colorectal | MEI-522(Voruciclib) | 1.1713 | 0.6952 | 176 | 5.14 | 12 |
| HCC1171 | Lung | MEI-522(Voruciclib) | 0.6742 | 0.7339 | 89 | 2.84 | 12 |
| HCC44 | Lung | MEI-522(Voruciclib) | 1.2105 | 0.7299 | 161 | 4.97 | 12 |
| HPAF-II | Pancreas | MEI-522(Voruciclib) | 1.3331 | 1.3400 | 97 | 2.84 | 12 |
| KYSE-410 | Esophageal | MEI-522(Voruciclib) | 1.7470 | 1.7986 | 87 | 2.41 | 12 |
| LS-513 | Colorectal | MEI-522(Voruciclib) | 0.8777 | 0.5292 | 147 | 4.51 | 12 |
| MIA PaCa-2 | Pancreas | MEI-522(Voruciclib) | 1.1328 | 0.8681 | 116 | 3.63 | 6 |
| NCl-H1373 | Lung | MEI-522(Voruciclib) | 1.3897 | 0.9715 | 147 | 4.24 | 6 |
| NCl-H1792 | Lung | MEI-522(Voruciclib) | 1.9143 | 1.4409 | 192 | 5.24 | 6 |
| NCl-H2030 | Lung | MEI-522(Voruciclib) | 1.6831 | 1.1259 | 188 | 5.28 | 6 |
| NCl-H23 | Lung | MEI-522(Voruciclib) | 1.7727 | 1.2602 | 181 | 5.00 | 6 |
| NCl-H358 | Lung | MEI-522(Voruciclib) | 0.5978 | 0.5850 | 137 | 3.61 | 12 |
| Panc 04.03 | Pancreas | MEI-522(Voruciclib) | 1.7425 | 1.2993 | 168 | 4.67 | 12 |
| SW48 | Colorectal | MEI-522(Voruciclib) | 0.5661 | 0.4222 | 135 | 4.44 | 12 |
| SW48 KRAS (G12C/+) | Colorectal | MEI-522(Voruciclib) | 0.7192 | 0.5860 | 115 | 3.67 | 12 |
| SW48 KRAS (G12D/+) | Colorectal | MEI-522(Voruciclib) | 0.5413 | 0.4283 | 139 | 4.36 | 12 |
| SW837 | Colorectal | MEI-522(Voruciclib) | 2.1000 | 1.7538 | 166 | 4.48 | 12 |
| TOV-21G | Ovary | MEI-522(Voruciclib) | 1.9759 | 1.5664 | 181 | 4.89 | 12 |
| AMG510 | | | | | | | |
| AsPC-1 | Pancreas | AMG510 | 3.0000 | | 8 | 0.00 | 3 |
| Calu-1 | Lung | AMG510 | 0.0029 | | 48 | 2.29 | 3 |
| Gp2D | Colorectal | AMG510 | 2.5503 | | 18 | 0.55 | 3 |
| HCC1171 | Lung | AMG510 | 0.0028 | 0.0055 | 85 | 3.50 | 3 |
| HCC44 | Lung | AMG510 | 0.0046 | | 41 | 1.97 | 3 |
| HPAF-II | Pancreas | AMG510 | 2.5398 | | 15 | 0.47 | 3 |
| KYSE-410 | Esophageal | AMG510 | 0.0060 | | 24 | 1.06 | 3 |
| LS-513 | Colorectal | AMG510 | 0.3202 | | 4 | −0.23 | 3 |
| MIA PaCa-2 | Pancreas | AMG510 | 0.0057 | 0.0092 | 77 | 3.77 | 3 |
| NCl-H1373 | Lung | AMG510 | 0.0021 | | 65 | 2.56 | 3 |
| NCl-H1792 | Lung | AMG510 | 0.0042 | | 37 | 1.34 | 3 |
| NCl-H2030 | Lung | AMG510 | 0.0044 | | 47 | 2.05 | 3 |
| NCl-H23 | Lung | AMG510 | 0.0067 | 0.0232 | 71 | 3.60 | 3 |
| NCl-H358 | Lung | AMG510 | 0.0094 | 0.0088 | 108 | 5.18 | 3 |
| Panc 04.03 | Pancreas | AMG510 | 3.0000 | | 25 | 0.00 | 3 |
| SW48 | Colorectal | AMG510 | 2.7165 | | 17 | 0.61 | 3 |
| SW48 KRAS (G12C/+) | Colorectal | AMG510 | 0.0021 | | 36 | 1.78 | 3 |
| SW48 KRAS (G12D/+) | Colorectal | AMG510 | 3.0000 | | 15 | 0.00 | 3 |
| SW837 | Colorectal | AMG510 | 0.0104 | 0.1617 | 56 | 2.67 | 3 |
| TOV-21G | Ovary | AMG510 | 3.0000 | | 9 | −0.01 | 3 |
| MRTX849 | | | | | | | |
| AsPC-1 | Pancreas | MRTX849 | 1.3682 | 1.5158 | 79 | 2.27 | 3 |
| Calu-1 | Lung | MRTX849 | 0.1756 | 0.1420 | 104 | 3.96 | 3 |
| Gp2D | Colorectal | MRTX849 | 2.2311 | 2.4408 | 67 | 1.86 | 3 |
| HCC1171 | Lung | MRTX849 | 0.0273 | 0.0113 | 138 | 5.95 | 3 |
| HCC44 | Lung | MRTX849 | 0.1811 | 0.3401 | 81 | 3.02 | 3 |
| HPAF-II | Pancreas | MRTX849 | 1.1168 | 1.2291 | 69 | 2.04 | 3 |
| KYSE-410 | Esophageal | MRTX849 | 1.3668 | 1.5538 | 77 | 2.24 | 3 |
| LS-513 | Colorectal | MRTX849 | 1.8352 | 2.4508 | 55 | 1.56 | 3 |
| MIA PaCa-2 | Pancreas | MRTX849 | 0.0079 | 0.0110 | 88 | 4.44 | 3 |
| NCl-H1373 | Lung | MRTX849 | 0.3768 | 0.1657 | 135 | 4.82 | 3 |
| NCl-H1792 | Lung | MRTX849 | 0.1826 | 0.3316 | 82 | 3.02 | 3 |
| NCl-H2030 | Lung | MRTX849 | 0.0302 | 0.2299 | 69 | 2.65 | 3 |
| NCl-H23 | Lung | MRTX849 | 0.0370 | 0.0430 | 96 | 4.15 | 3 |
| NCl-H358 | Lung | MRTX849 | 0.0685 | 0.0294 | 149 | 5.85 | 3 |
| Panc 04.03 | Pancreas | MRTX849 | 1.9962 | 2.0890 | 81 | 2.20 | 3 |
| SW48 | Colorectal | MRTX849 | 1.1909 | 1.2165 | 95 | 2.78 | 3 |
| SW48 KRAS (G12C/+) | Colorectal | MRTX849 | 0.2173 | 0.4612 | 77 | 2.88 | 3 |
| SW48 KRAS (G12D/+) | Colorectal | MRTX849 | 1.1845 | 1.2771 | 87 | 2.56 | 3 |
| SW837 | Colorectal | MRTX849 | 0.4372 | 0.1754 | 166 | 5.55 | 3 |
| TOV-21G | Ovary | MRTX849 | 1.4266 | 1.5546 | 80 | 2.28 | 3 |
| Onvansertib | | | | | | | |
| AsPC-1 | Pancreas | Onvansertib | 0.0823 | 0.1092 | 84 | 3.48 | 3 |
| Gp2D | Colorectal | Onvansertib | 0.0339 | 0.0224 | 170 | 7.46 | 3 |
| HCC1171 | Lung | Onvansertib | 0.0155 | 0.0330 | 81 | 3.02 | 3 |
| HCC44 | Lung | Onvansertib | 0.0220 | 0.0187 | 112 | 5.16 | 3 |
| HPAF-II | Pancreas | Onvansertib | 0.0210 | 0.0242 | 87 | 3.98 | 3 |
| KYSE-410 | Esophageal | Onvansertib | 0.0347 | 0.0431 | 78 | 3.49 | 3 |
| LS-513 | Colorectal | Onvansertib | 0.0494 | 0.0517 | 97 | 4.03 | 3 |
| NCl-H358 | Lung | Onvansertib | 0.0182 | 0.0183 | 127 | 4.69 | 3 |
| Panc 04.03 | Pancreas | Onvansertib | 0.0192 | 0.0508 | 70 | 2.60 | 3 |
| SW48 | Colorectal | Onvansertib | 0.0154 | 0.0134 | 177 | 7.09 | 3 |
| SW48 KRAS (G12C/+) | Colorectal | Onvansertib | 0.0171 | 0.0159 | 159 | 5.77 | 3 |
| SW48 KRAS (G12D/+) | Colorectal | Onvansertib | 0.0157 | 0.0139 | 168 | 5.57 | 3 |
| SW837 | Colorectal | Onvansertib | 0.0421 | 0.0341 | 118 | 5.06 | 3 |
| TOV-21G | Ovary | Onvansertib | 0.0342 | 0.0265 | 143 | 6.09 | 3 |
| Volasertib | | | | | | | |
| AsPC-1 | Pancreas | Volasertib | 0.0122 | 0.0137 | 96 | 4.36 | 3 |
| Gp2D | Colorectal | Volasertib | 0.0025 | 0.0022 | 161 | 6.83 | 3 |
| HCC1171 | Lung | Volasertib | 0.0047 | 0.0053 | 79 | 3.40 | 3 |
| HCC44 | Lung | Volasertib | 0.0045 | 0.0044 | 113 | 5.39 | 3 |
| HPAF-II | Pancreas | Volasertib | 0.0039 | 0.0044 | 95 | 4.65 | 3 |
| KYSE-410 | Esophageal | Volasertib | 0.0085 | 0.0101 | 89 | 4.24 | 3 |
| LS-513 | Colorectal | Volasertib | 0.0185 | 0.0200 | 91 | 4.22 | 3 |
| NCl-H358 | Lung | Volasertib | 0.0061 | 0.0065 | 100 | 4.57 | 3 |
| Panc 04.03 | Pancreas | Volasertib | 0.0042 | 0.0046 | 87 | 3.75 | 3 |
| SW48 | Colorectal | Volasertib | 0.0049 | 0.0046 | 174 | 6.76 | 3 |
| SW48 KRAS (G12C/+) | Colorectal | Volasertib | 0.0044 | 0.0044 | 141 | 5.48 | 3 |
| SW48 KRAS (G12D/+) | Colorectal | Volasertib | 0.0042 | 0.0043 | 148 | 5.01 | 3 |
| SW837 | Colorectal | Volasertib | 0.0201 | 0.0148 | 128 | 5.63 | 3 |
| TOV-21G | Ovary | Volasertib | 0.0138 | 0.0084 | 163 | 7.56 | 3 |

Additional Single Agent Responses

| | | | EC₅₀ | IC₅₀ | Max Response | Response | |
|---|---|---|---|---|---|---|---|
| Cell Line | Tissue | Compound | (μM) | (μM) | (% I) | Area | N |
| AsPC-1 | Pancreas | MEI-522(Voruciclib) | 1.3338 | 2.3195 | 68 | 1.96 | 12 |
| Calu-1 | Lung | MEI-522(Voruciclib) | 1.8591 | 1.9806 | 90 | 2.48 | 6 |
| Gp2D | Colorectal | MEI-522(Voruciclib) | 0.7467 | 0.7910 | 94 | 2.94 | 12 |
| HCC1171 | Lung | MEI-522(Voruciclib) | 0.6800 | 3.0301 | 51 | 1.62 | 12 |
| HCC44 | Lung | MEI-522(Voruciclib) | 0.7699 | 0.8291 | 91 | 2.85 | 12 |
| HPAF-II | Pancreas | MEI-522(Voruciclib) | 1.3140 | 1.8828 | 72 | 2.09 | 12 |
| KYSE-410 | Esophageal | MEI-522(Voruciclib) | 1.7442 | 1.8966 | 72 | 1.97 | 12 |
| LS-513 | Colorectal | MEI-522(Voruciclib) | 0.5493 | 0.6330 | 90 | 2.94 | 12 |
| MIA PaCa-2 | Pancreas | MEI-522(Voruciclib) | 0.8940 | 1.0655 | 85 | 2.64 | 6 |
| NCl-H1373 | Lung | MEI-522(Voruciclib) | 1.0303 | 1.2325 | 86 | 2.58 | 6 |
| NCl-H1792 | Lung | MEI-522(Voruciclib) | 1.3530 | 1.3600 | 98 | 2.83 | 6 |
| NCl-H2030 | Lung | MEI-522(Voruciclib) | 1.0483 | 1.0627 | 98 | 2.92 | 6 |
| NCl-H23 | Lung | MEI-522(Voruciclib) | 1.1326 | 1.1750 | 96 | 2.83 | 6 |
| NCl-H358 | Lung | MEI-522(Voruciclib) | 0.5914 | 0.6275 | 85 | 2.50 | 12 |
| Panc 04.03 | Pancreas | MEI-522(Voruciclib) | 1.3113 | 1.4068 | 92 | 2.65 | 12 |
| SW48 | Colorectal | MEI-522(Voruciclib) | 0.4434 | 0.5255 | 87 | 2.91 | 12 |
| SW48 KRAS | Colorectal | MEI-522(Voruciclib) | 0.5909 | 0.7314 | 85 | 2.74 | 12 |
| (G12C/+) | | | | | | | |
| SW48 KRAS | Colorectal | MEI-522(Voruciclib) | 0.4628 | 0.5721 | 84 | 2.81 | 12 |
| (G12D/+) | | | | | | | |
| SW837 | Colorectal | MEI-522(Voruciclib) | 1.9904 | 2.0891 | 90 | 2.44 | 12 |
| TOV-21G | Ovary | MEI-522(Voruciclib) | 1.6618 | 1.7280 | 95 | 2.63 | 12 |
| AsPC-1 | Pancreas | AMG510 | 3.0000 | | 6 | 0.00 | 3 |
| Calu-1 | Lung | AMG510 | 0.0028 | | 37 | 1.72 | 3 |
| Gp2D | Colorectal | AMG510 | 2.5642 | | 13 | 0.42 | 3 |
| HCC1171 | Lung | AMG510 | 0.0028 | | 48 | 1.99 | 3 |
| HCC44 | Lung | AMG510 | 0.0041 | | 32 | 1.52 | 3 |
| HPAF-II | Pancreas | AMG510 | 2.4015 | | 11 | 0.32 | 3 |
| KYSE-410 | Esophageal | AMG510 | 0.0058 | | 20 | 0.88 | 3 |
| LS-513 | Colorectal | AMG510 | 0.3209 | | 3 | −0.19 | 3 |
| MIA PaCa-2 | Pancreas | AMG510 | 0.0057 | 0.0147 | 63 | 3.12 | 3 |
| NCl-H1373 | Lung | AMG510 | 0.0025 | | 49 | 2.05 | 3 |
| NCl-H1792 | Lung | AMG510 | 0.0042 | | 29 | 1.05 | 3 |
| NCl-H2030 | Lung | AMG510 | 0.0043 | | 38 | 1.65 | 3 |
| NCl-H23 | Lung | AMG510 | 0.0067 | 0.1567 | 56 | 2.82 | 3 |
| NCl-H358 | Lung | AMG510 | 0.0085 | 0.0142 | 79 | 3.87 | 3 |
| Panc 04.03 | Pancreas | AMG510 | 3.0000 | | 19 | 0.00 | 3 |
| SW48 | Colorectal | AMG510 | 2.7089 | | 14 | 0.48 | 3 |
| SW48 KRAS | Colorectal | AMG510 | 0.0021 | | 29 | 1.47 | 3 |
| (G12C/+) | | | | | | | |
| SW48 KRAS | Colorectal | AMG510 | 3.0000 | | 11 | 0.00 | 3 |
| (G12D/+) | | | | | | | |
| SW837 | Colorectal | AMG510 | 0.0099 | | 37 | 1.79 | 3 |
| TOV-21G | Ovary | AMG510 | 3.0000 | | 7 | −0.01 | 3 |
| AsPC-1 | Pancreas | MRTX849 | 1.4074 | 2.3723 | 53 | 1.54 | 3 |
| Calu-1 | Lung | MRTX849 | 0.1483 | 0.3956 | 75 | 2.89 | 3 |
| Gp2D | Colorectal | MRTX849 | 2.2311 | 2.9156 | 51 | 1.41 | 3 |
| HCC1171 | Lung | MRTX849 | 0.0210 | 0.1060 | 73 | 3.24 | 3 |
| HCC44 | Lung | MRTX849 | 0.1909 | 1.0055 | 65 | 2.39 | 3 |
| HPAF-II | Pancreas | MRTX849 | 1.1162 | 1.8320 | 50 | 1.48 | 3 |
| KYSE-410 | Esophageal | MRTX849 | 1.4329 | 1.8713 | 65 | 1.94 | 3 |
| LS-513 | Colorectal | MRTX849 | 1.9059 | | 45 | 1.29 | 3 |
| MIA PaCa-2 | Pancreas | MRTX849 | 0.0078 | 0.0206 | 72 | 3.66 | 3 |
| NCl-H1373 | Lung | MRTX849 | 0.1887 | 0.3349 | 82 | 3.09 | 3 |
| NCl-H1792 | Lung | MRTX849 | 0.2016 | 0.9080 | 65 | 2.42 | 3 |
| NCl-H2030 | Lung | MRTX849 | 0.0301 | | 54 | 2.10 | 3 |
| NCl-H23 | Lung | MRTX849 | 0.0349 | 0.1062 | 73 | 3.19 | 3 |
| NCl-H358 | Lung | MRTX849 | 0.0282 | 0.0456 | 86 | 3.71 | 3 |
| Panc 04.03 | Pancreas | MRTX849 | 2.0885 | 2.4106 | 60 | 1.65 | 3 |
| SW48 | Colorectal | MRTX849 | 1.2280 | 1.4525 | 75 | 2.21 | 3 |
| SW48 KRAS | Colorectal | MRTX849 | 0.2176 | 1.1172 | 64 | 2.38 | 3 |
| (G12C/+) | | | | | | | |
| SW48 KRAS | Colorectal | MRTX849 | 1.2224 | 1.7122 | 65 | 1.96 | 3 |
| (G12D/+) | | | | | | | |
| SW837 | Colorectal | MRTX849 | 0.2696 | 0.3343 | 90 | 3.22 | 3 |
| TOV-21G | Ovary | MRTX849 | 1.5202 | 2.1496 | 57 | 1.66 | 3 |
| AsPC-1 | Pancreas | Onvansertib | 0.0949 | 0.3319 | 60 | 2.50 | 3 |
| Gp2D | Colorectal | Onvansertib | 0.0238 | 0.0255 | 93 | 4.29 | 3 |
| HCC1171 | Lung | Onvansertib | 0.0155 | | 46 | 1.75 | 3 |
| HCC44 | Lung | Onvansertib | 0.0185 | 0.0253 | 81 | 3.83 | 3 |
| HPAF-II | Pancreas | Onvansertib | 0.0209 | 0.0359 | 65 | 2.97 | 3 |
| KYSE-410 | Esophageal | Onvansertib | 0.0349 | 0.0555 | 65 | 2.89 | 3 |
| LS-513 | Colorectal | Onvansertib | 0.0494 | 0.0616 | 79 | 3.29 | 3 |
| NCl-H358 | Lung | Onvansertib | 0.0167 | 0.0203 | 82 | 3.36 | 3 |
| Panc 04.03 | Pancreas | Onvansertib | 0.0187 | | 54 | 1.94 | 3 |
| SW48 | Colorectal | Onvansertib | 0.0138 | 0.0143 | 95 | 4.36 | 3 |
| SW48 KRAS | Colorectal | Onvansertib | 0.0155 | 0.0163 | 93 | 4.14 | 3 |
| (G12C/+) | | | | | | | |
| SW48 KRAS | Colorectal | Onvansertib | 0.0140 | 0.0178 | 92 | 3.73 | 3 |
| (G12D/+) | | | | | | | |
| SW837 | Colorectal | Onvansertib | 0.0354 | 0.0534 | 76 | 3.32 | 3 |
| TOV-21G | Ovary | Onvansertib | 0.0281 | 0.0316 | 86 | 3.84 | 3 |
| AsPC-1 | Pancreas | Volasertib | 0.0122 | 0.0231 | 69 | 3.11 | 3 |
| Gp2D | Colorectal | Volasertib | 0.0021 | 0.0024 | 91 | 4.53 | 3 |
| HCC1171 | Lung | Volasertib | 0.0047 | | 45 | 1.99 | 3 |
| HCC44 | Lung | Volasertib | 0.0042 | 0.0063 | 81 | 4.07 | 3 |
| HPAF-II | Pancreas | Volasertib | 0.0039 | 0.0062 | 71 | 3.45 | 3 |
| KYSE-410 | Esophageal | Volasertib | 0.0086 | 0.0131 | 74 | 3.52 | 3 |
| LS-513 | Colorectal | Volasertib | 0.0186 | 0.0239 | 74 | 3.44 | 3 |
| NCl-H358 | Lung | Volasertib | 0.0061 | 0.0078 | 75 | 3.44 | 3 |
| Panc 04.03 | Pancreas | Volasertib | 0.0042 | 0.0059 | 64 | 2.78 | 3 |
| SW48 | Colorectal | Volasertib | 0.0047 | 0.0049 | 95 | 4.47 | 3 |
| SW48 KRAS | Colorectal | Volasertib | 0.0043 | 0.0046 | 90 | 3.98 | 3 |
| (G12C/+) | | | | | | | |
| SW48 KRAS | Colorectal | Volasertib | 0.0041 | 0.0048 | 86 | 3.32 | 3 |
| (G12D/+) | | | | | | | |
| SW837 | Colorectal | Volasertib | 0.0138 | 0.0210 | 79 | 3.61 | 3 |
| TOV-21G | Ovary | Volasertib | 0.0100 | 0.0116 | 89 | 4.33 | 3 |

### Combination Responses

TABLE 2A
Combination Data Metrics for MEI-522 (Voruciclib) across six cell lines. Synergy scores
as determined by Chalice with Bliss, HSA and Loewe Volume scores for the growth inhibition
dose matrices. Data metrics were determined by chalice and reported where determined.

| | | | Synergy | Best | Best CI | Bliss | HSA | Loewe | |
|---|---|---|---|---|---|---|---|---|---|
| Cell Line | Tissue | Enhancer | Score | CI | Level | Volume | volume | Volume | N |
| AsPC-1 | Pancreas | AMG510 | 0.82 | 0.47 | 20 | 2.91 | −3.63 | 0.38 | 3 |
| Calu-1 | Lung | AMG510 | 4.52 | 0.80 | 75 | 2.90 | 2.88 | 2.66 | 3 |
| Gp2D | Colorectal | AMG510 | 2.14 | 0.47 | 175 | 1.60 | −1.47 | 1.40 | 3 |
| HCC1171 | Lung | AMG510 | 7.42 | 0.26 | 95 | 3.26 | 4.61 | 6.02 | 3 |
| HCC44 | Lung | AMG510 | 4.36 | 0.63 | 140 | 2.46 | 2.64 | 2.30 | 3 |
| HPAF-II | Pancreas | AMG510 | 0.12 | 0.06 | 20 | −3.31 | −4.44 | −3.46 | 3 |
| KYSE-410 | Esophageal | AMG510 | 1.71 | 1.14 | 55 | 1.47 | 2.26 | 2.48 | 3 |
| LS-513 | Colorectal | AMG510 | 1.51 | 0.70 | 20 | 0.20 | −2.06 | −0.76 | 3 |
| MIA PaCa-2 | Pancreas | AMG510 | 6.04 | 0.65 | 115 | 3.75 | 5.03 | 4.68 | 3 |
| NCl-H1373 | Lung | AMG510 | 4.10 | 0.28 | 25 | 2.91 | 1.67 | 2.62 | 3 |
| NCl-H1792 | Lung | AMG510 | 3.62 | 0.36 | 45 | 5.76 | 3.59 | 4.64 | 3 |
| NCl-H2030 | Lung | AMG510 | 5.13 | 0.47 | 60 | 6.35 | 4.90 | 4.51 | 3 |
| NCl-H23 | Lung | AMG510 | 8.27 | 0.76 | 125 | 8.97 | 6.92 | 6.28 | 3 |
| NCl-H358 | Lung | AMG510 | 3.63 | 0.51 | 25 | 4.55 | 2.53 | 2.07 | 3 |
| Panc 04.03 | Pancreas | AMG510 | 3.01 | 0.51 | 45 | 2.52 | −2.59 | 2.70 | 3 |
| SW48 | Colorectal | AMG510 | 4.05 | 0.46 | 45 | 2.71 | 0.73 | 2.40 | 3 |
| SW48 KRAS | Colorectal | AMG510 | 5.97 | 0.36 | 130 | 2.79 | 5.42 | 4.73 | 3 |
| (G12C/+) | | | | | | | | | |
| SW48 KRAS | Colorectal | AMG510 | 2.74 | 0.55 | 130 | 0.17 | −6.22 | 0.13 | 3 |
| (G12D/+) | | | | | | | | | |
| SW837 | Colorectal | AMG510 | 6.03 | 0.72 | 95 | 7.24 | 6.16 | 6.02 | 3 |
| TOV-21G | Ovary | AMG510 | 0.49 | 0.37 | 25 | 2.49 | −6.11 | −3.64 | 3 |
| AsPC-1 | Pancreas | MRTX849 | 1.74 | 0.34 | 20 | 1.59 | −0.92 | 0.71 | 3 |
| Calu-1 | Lung | MRTX849 | 5.59 | 0.67 | 45 | 4.36 | 1.87 | 1.52 | 3 |
| Gp2D | Colorectal | MRTX849 | 1.80 | 0.50 | 20 | 1.83 | 0.16 | 0.46 | 3 |
| HCC1171 | Lung | MRTX849 | 5.14 | 0.48 | 110 | 2.95 | 3.86 | 2.20 | 3 |
| HCC44 | Lung | MRTX849 | 10.87 | 0.62 | 150 | 8.29 | 6.89 | 6.58 | 3 |
| HPAF-II | Pancreas | MRTX849 | 1.51 | 0.87 | 20 | 0.87 | 1.34 | −0.08 | 3 |
| KYSE-410 | Esophageal | MRTX849 | 2.19 | 0.30 | 25 | 7.25 | 2.25 | 6.19 | 3 |
| LS-513 | Colorectal | MRTX849 | 2.44 | 0.59 | 25 | 1.48 | 0.51 | 0.85 | 3 |
| MIA PaCa-2 | Pancreas | MRTX849 | 6.62 | 0.85 | 140 | 4.09 | 5.25 | 4.29 | 3 |
| NCl-H1373 | Lung | MRTX849 | 7.91 | 0.81 | 145 | 5.61 | 2.06 | 3.23 | 3 |
| NCl-H1792 | Lung | MRTX849 | 5.41 | 1.34 | 140 | 7.60 | 4.71 | 5.13 | 3 |
| NCl-H2030 | Lung | MRTX849 | 3.68 | 0.68 | 20 | 3.47 | 1.90 | 0.97 | 3 |
| NCl-H23 | Lung | MRTX849 | 7.32 | 1.07 | 155 | 8.21 | 5.10 | 4.80 | 3 |
| NCl-H358 | Lung | MRTX849 | 4.32 | 0.41 | 140 | 5.55 | 2.90 | 1.20 | 3 |
| Panc 04.03 | Pancreas | MRTX849 | 2.94 | 1.36 | 120 | 4.21 | 0.64 | 3.31 | 3 |
| SW48 | Colorectal | MRTX849 | 5.80 | 0.57 | 40 | 9.14 | 3.40 | 6.95 | 3 |
| SW48 KRAS | Colorectal | MRTX849 | 5.83 | 0.48 | 105 | 3.57 | 4.53 | 4.75 | 3 |
| (G12C/+) | | | | | | | | | |
| SW48 KRAS | Colorectal | MRTX849 | 3.96 | 0.46 | 140 | 3.29 | 0.06 | 1.71 | 3 |
| (G12D/+) | | | | | | | | | |
| SW837 | Colorectal | MRTX849 | 7.79 | 0.47 | 50 | 10.52 | 6.87 | 5.57 | 3 |
| TOV-21G | Ovary | MRTX849 | 2.13 | 0.40 | 30 | 3.33 | −2.03 | 1.51 | 3 |
| AsPC-1 | Pancreas | Onvansertib | 1.41 | 0.84 | 20 | −1.25 | −1.39 | −0.75 | 3 |
| Gp2D | Colorectal | Onvansertib | 3.16 | 0.96 | 175 | 7.51 | −1.36 | −1.41 | 3 |
| HCC1171 | Lung | Onvansertib | 1.86 | 1.01 | 90 | −2.83 | −2.66 | −1.72 | 3 |
| HCC44 | Lung | Onvansertib | 3.96 | 0.65 | 145 | 4.26 | 1.33 | 0.16 | 3 |
| HPAF-II | Pancreas | Onvansertib | 1.29 | 0.70 | 20 | −0.18 | 0.48 | −0.22 | 3 |
| KYSE-410 | Esophageal | Onvansertib | 0.29 | 0.60 | 20 | −1.39 | −2.18 | −2.01 | 3 |
| LS-513 | Colorectal | Onvansertib | 2.51 | 0.96 | 150 | 0.53 | −0.64 | −1.49 | 3 |
| NCl-H358 | Lung | Onvansertib | 6.46 | 0.27 | 110 | −1.00 | −5.36 | −3.73 | 3 |
| Panc 04.03 | Pancreas | Onvansertib | 1.99 | 0.32 | 20 | 2.32 | 0.85 | 0.87 | 3 |
| SW48 | Colorectal | Onvansertib | 7.50 | 0.72 | 145 | 6.27 | −3.43 | −1.93 | 3 |
| SW48 KRAS | Colorectal | Onvansertib | 5.28 | 0.86 | 120 | 2.74 | −2.16 | −0.91 | 3 |
| (G12C/+) | | | | | | | | | |
| SW48 KRAS | Colorectal | Onvansertib | 8.15 | 0.39 | 125 | 5.34 | −1.32 | 1.06 | 3 |
| (G12D/+) | | | | | | | | | |
| SW837 | Colorectal | Onvansertib | 0.88 | 0.78 | 30 | 0.66 | −1.30 | −4.09 | 3 |
| TOV-21G | Ovary | Onvansertib | 1.56 | 0.40 | 40 | 5.55 | −0.24 | −0.71 | 3 |
| AsPC-1 | Pancreas | Volasertib | 1.87 | 0.51 | 95 | −1.02 | 0.04 | −0.85 | 3 |
| Gp2D | Colorectal | Volasertib | 15.26 | 0.59 | 175 | 9.40 | 0.44 | 1.73 | 3 |
| HCC1171 | Lung | Volasertib | 2.07 | 0.95 | 85 | −2.15 | −1.49 | −1.27 | 3 |
| HCC44 | Lung | Volasertib | 6.97 | 0.55 | 160 | 6.88 | 2.51 | 0.65 | 3 |
| HPAF-II | Pancreas | Volasertib | 2.64 | 0.66 | 100 | −0.85 | 0.04 | −0.80 | 3 |
| KYSE-410 | Esophageal | Volasertib | 0.91 | 1.85 | 80 | −3.04 | −2.10 | −3.58 | 3 |
| LS-513 | Colorectal | Volasertib | 5.33 | 0.60 | 145 | 3.16 | 1.53 | 1.21 | 3 |
| NCl-H358 | Lung | Volasertib | 3.21 | | 110 | −1.58 | −4.12 | −3.93 | 3 |
| Panc 04.03 | Pancreas | Volasertib | 7.71 | 0.74 | 170 | 4.14 | 2.74 | 1.36 | 3 |
| SW48 | Colorectal | Volasertib | 11.75 | 0.11 | 140 | 8.81 | −0.59 | 1.23 | 3 |
| SW48 KRAS | Colorectal | Volasertib | 11.77 | 0.08 | 125 | 5.42 | 1.24 | 2.81 | 3 |
| (G12C/+) | | | | | | | | | |
| SW48 KRAS | Colorectal | Volasertib | 13.34 | 0.06 | 135 | 8.36 | 1.07 | 3.66 | 3 |
| (G12D/+) | | | | | | | | | |
| SW837 | Colorectal | Volasertib | 5.42 | 0.72 | 160 | 4.69 | 2.14 | 0.39 | 3 |
| TOV-21G | Ovary | Volasertib | 1.71 | 0.56 | 20 | 2.45 | −4.18 | −6.03 | 3 |

TABLE 2B
Combination Data Metrics for MEI-522 (Voruciclib) across six cell lines. Inhibition data

| | | | Synergy | Best | Best CI | Bliss | HSA | Loewe | |
|---|---|---|---|---|---|---|---|---|---|
| Cell Line | Tissue | Enhancer | Score | CI | Level | Volume | volume | Volume | N |
| AsPC-1 | Pancreas | AMG510 | 0.32 | 0.64 | 20 | 1.95 | −2.52 | 0.26 | 3 |
| Calu-1 | Lung | AMG510 | 1.42 | 0.72 | 55 | −0.15 | 1.55 | 1.28 | 3 |
| Gp2D | Colorectal | AMG510 | 0.43 | 0.90 | 20 | 0.54 | −1.21 | 0.47 | 3 |
| HCC1171 | Lung | AMG510 | 2.15 | 0.22 | 50 | −0.99 | 2.26 | 3.06 | 3 |
| HCC44 | Lung | AMG510 | 1.16 | 0.67 | 45 | −0.82 | 1.08 | 0.97 | 3 |
| HPAF-II | Pancreas | AMG510 | 0.09 | 1.03 | 20 | −2.30 | −3.13 | −2.28 | 3 |
| KYSE-410 | Esophageal | AMG510 | 1.19 | 1.14 | 45 | 1.06 | 1.87 | 2.13 | 3 |
| LS-513 | Colorectal | AMG510 | 0.37 | 0.82 | 20 | −0.02 | −1.47 | −0.89 | 3 |
| MIA PaCa-2 | Pancreas | AMG510 | 2.16 | 0.60 | 75 | 0.19 | 2.76 | 2.52 | 3 |
| NCl-H1373 | Lung | AMG510 | 1.25 | 0.93 | 20 | −1.68 | 0.90 | 0.62 | 3 |
| NCl-H1792 | Lung | AMG510 | 1.87 | 0.46 | 35 | 2.25 | 2.86 | 3.31 | 3 |
| NCl-H2030 | Lung | AMG510 | 2.04 | 0.70 | 50 | 1.77 | 3.51 | 3.26 | 3 |
| NCl-H23 | Lung | AMG510 | 3.46 | 0.53 | 80 | 1.69 | 4.54 | 3.98 | 3 |
| NCl-H358 | Lung | AMG510 | 1.07 | 0.51 | 20 | −0.42 | 1.90 | 1.20 | 3 |
| Panc 04.03 | Pancreas | AMG510 | 1.06 | 0.60 | 20 | 0.79 | −2.11 | 1.18 | 3 |
| SW48 | Colorectal | AMG510 | 1.36 | 0.50 | 35 | 1.20 | 0.35 | 1.24 | 3 |
| SW48 KRAS | Colorectal | AMG510 | 2.77 | 0.62 | 60 | 0.42 | 3.57 | 3.31 | 3 |
| (G12C/+) | | | | | | | | | |
| SW48 KRAS | Colorectal | AMG510 | 0.86 | 1.38 | 20 | −0.98 | −4.35 | −0.76 | 3 |
| (G12D/+) | | | | | | | | | |
| SW837 | Colorectal | AMG510 | 2.77 | 0.64 | 65 | 2.98 | 4.27 | 4.27 | 3 |
| TOV-21G | Ovary | AMG510 | 0.33 | 0.45 | 20 | 2.55 | −4.28 | −2.46 | 3 |
| AsPC-1 | Pancreas | MRTX849 | 0.72 | 0.58 | 20 | 0.56 | −0.66 | 0.37 | 3 |
| Calu-1 | Lung | MRTX849 | 1.80 | 0.54 | 30 | −0.16 | 0.51 | 0.40 | 3 |
| Gp2D | Colorectal | MRTX849 | 0.48 | 0.60 | 20 | 0.59 | −0.16 | 0.37 | 3 |
| HCC1171 | Lung | MRTX849 | 1.36 | 0.51 | 60 | −1.94 | 1.80 | 0.93 | 3 |
| HCC44 | Lung | MRTX849 | 2.02 | 0.72 | 35 | 1.78 | 2.81 | 2.31 | 3 |
| HPAF-II | Pancreas | MRTX849 | 0.71 | 0.84 | 20 | 0.27 | 0.93 | −0.16 | 3 |
| KYSE-410 | Esophageal | MRTX849 | 1.53 | 0.20 | 20 | 5.69 | 1.80 | 5.14 | 3 |
| LS-513 | Colorectal | MRTX849 | 0.56 | 0.59 | 20 | 0.42 | 0.34 | 0.46 | 3 |
| MIA PaCa-2 | Pancreas | MRTX849 | 2.24 | 0.36 | 60 | −0.43 | 2.97 | 2.30 | 3 |
| NCl-H1373 | Lung | MRTX849 | 1.94 | 0.19 | 20 | −0.55 | 1.03 | 1.11 | 3 |
| NCl-H1792 | Lung | MRTX849 | 2.48 | 1.09 | 80 | 2.56 | 3.40 | 3.37 | 3 |
| NCl-H2030 | Lung | MRTX849 | 0.96 | 0.60 | 20 | −0.64 | 1.28 | 0.45 | 3 |
| NCl-H23 | Lung | MRTX849 | 2.62 | 0.60 | 80 | 0.81 | 2.97 | 2.46 | 3 |
| NCl-H358 | Lung | MRTX849 | 0.99 | 0.58 | 20 | −0.46 | 1.91 | 0.52 | 3 |
| Panc 04.03 | Pancreas | MRTX849 | 0.94 | 1.35 | 75 | 1.85 | 0.37 | 1.76 | 3 |
| SW48 | Colorectal | MRTX849 | 2.31 | 0.76 | 35 | 4.79 | 2.30 | 4.49 | 3 |
| SW48 KRAS | Colorectal | MRTX849 | 2.49 | 0.32 | 25 | 1.09 | 2.63 | 2.96 | 3 |
| (G12C/+) | | | | | | | | | |
| SW48 KRAS | Colorectal | MRTX849 | 1.12 | 0.87 | 20 | 0.47 | −0.29 | 0.54 | 3 |
| (G12D/+) | | | | | | | | | |
| SW837 | Colorectal | MRTX849 | 2.22 | 0.11 | 30 | 4.03 | 4.50 | 3.46 | 3 |
| TOV-21G | Ovary | MRTX849 | 0.92 | 0.50 | 20 | 0.98 | −1.59 | 0.52 | 3 |
| AsPC-1 | Pancreas | Onvansertib | 0.67 | 1.13 | 20 | −1.91 | −0.98 | −0.67 | 3 |
| Gp2D | Colorectal | Onvansertib | 0.56 | 0.77 | 20 | 0.17 | 0.25 | 0.05 | 3 |
| HCC1171 | Lung | Onvansertib | 0.59 | 0.62 | 50 | −3.14 | −1.61 | −1.03 | 3 |
| HCC44 | Lung | Onvansertib | 0.67 | 0.80 | 20 | −0.85 | 0.68 | −0.34 | 3 |
| HPAF-II | Pancreas | Onvansertib | 0.74 | 0.85 | 20 | −1.20 | 0.37 | −0.12 | 3 |
| KYSE-410 | Esophageal | Onvansertib | 0.20 | 0.68 | 20 | −1.63 | −1.72 | −1.53 | 3 |
| LS-513 | Colorectal | Onvansertib | 0.70 | 0.90 | 20 | −2.22 | −0.87 | −1.32 | 3 |
| NCI-H358 | Lung | Onvansertib | 1.36 | 0.38 | 75 | −3.69 | −3.63 | −3.23 | 3 |
| Panc 04.03 | Pancreas | Onvansertib | 0.98 | 0.64 | 20 | 0.17 | 0.61 | 0.89 | 3 |
| SW48 | Colorectal | Onvansertib | 1.28 | 0.76 | 20 | −0.78 | −0.51 | −0.74 | 3 |
| SW48 KRAS | Colorectal | Onvansertib | 1.03 | 0.67 | 20 | −0.08 | 0.19 | −0.06 | 3 |
| (G12C/+) | | | | | | | | | |
| SW48 KRAS | Colorectal | Onvansertib | 2.12 | 0.51 | 20 | −0.39 | −0.28 | 0.65 | 3 |
| (G12D/+) | | | | | | | | | |
| SW837 | Colorectal | Onvansertib | 0.33 | 0.76 | 20 | −1.39 | −0.59 | −2.03 | 3 |
| TOV-21G | Ovary | Onvansertib | 0.98 | 0.56 | 30 | 0.52 | 0.33 | 0.43 | 3 |
| AsPC-1 | Pancreas | Volasertib | 0.83 | 0.89 | 20 | −2.24 | −0.18 | −0.80 | 3 |
| Gp2D | Colorectal | Volasertib | 2.39 | 0.16 | 80 | −1.26 | −0.27 | −0.15 | 3 |
| HCC1171 | Lung | Volasertib | 0.73 | 0.79 | 50 | −3.18 | −0.81 | −0.71 | 3 |
| HCC44 | Lung | Volasertib | 1.13 | 0.83 | 80 | −1.88 | 0.12 | −0.92 | 3 |
| HPAF-II | Pancreas | Volasertib | 0.98 | 0.52 | 70 | −2.61 | −0.36 | −0.96 | 3 |
| KYSE-410 | Esophageal | Volasertib | 0.62 | 0.40 | 70 | −3.11 | −1.71 | −2.95 | 3 |
| LS-513 | Colorectal | Volasertib | 1.44 | 0.62 | 80 | −1.28 | 0.04 | 0.06 | 3 |
| NCl-H358 | Lung | Volasertib | 0.69 | 0.80 | 20 | −4.08 | −3.08 | −3.28 | 3 |
| Panc 04.03 | Pancreas | Volasertib | 2.30 | 0.67 | 80 | −0.87 | 1.03 | 0.18 | 3 |
| SW48 | Colorectal | Volasertib | 1.83 | 0.60 | 20 | 0.08 | 0.40 | 0.43 | 3 |
| SW48 KRAS | Colorectal | Volasertib | 3.33 | 0.13 | 80 | −0.04 | 0.65 | 1.39 | 3 |
| (G12C/+) | | | | | | | | | |
| SW48 KRAS | Colorectal | Volasertib | 4.06 | 0.08 | 80 | 0.41 | 0.61 | 2.08 | 3 |
| (G12D/+) | | | | | | | | | |
| SW837 | Colorectal | Volasertib | 1.39 | 0.83 | 20 | 0.17 | 1.01 | 0.29 | 3 |
| TOV-21G | Ovary | Volasertib | 0.60 | 0.76 | 20 | −1.60 | −1.29 | −1.80 | 3 |

Protocol

The method for High Throughput Screen is described below. The endpoint readout of this assay is based upon quantitation of ATP as an indicator of viable cells.

Cell lines that have been preserved in liquid nitrogen are thawed and expanded in growth media (see Appendix I). Once cells have reached expected doubling times, screening begins. Cells are seeded in 25 μl of growth media in black 384-well tissue culture treated plates at 500 cells per well (as noted in Analyzer). Cells are equilibrated in assay plates via centrifugation and placed at 37° C. 5% CO₂ for twenty-four hours before treatment. At the time of treatment, a set of assay plates (which do not receive treatment) are collected and ATP levels are measured by adding CellTiter-Glo 2.0 (Promega) and luminescence read on Envision plate readers (Perkin Elmer). Compounds are transferred to assay plates using and Echo acoustic liquid handling system. 25 μl of each compound was added at the appropriate concentration for all combination dose points. Therefore, final assay volume would be 25.05 μl. Assay plates are incubated with compound for 3 days and are then analyzed using CellTiter-Glo 2.0. All data points are collected via automated processes and are subject to quality control and analyzed.

Growth Inhibition (GI) is used as a measure of cell growth. The GI percentages are calculated by applying the following test and equation:

If T<V_0:100*(1−(T−V_0)/V_0)

If T≥V_0:100*(1−(T−V_0)/(V−V_0))
where T is the signal measure for a test article, V is the untreated/vehicle-treated control measure, and Vo is the untreated/vehicle control measure at time zero (also colloquially referred as TO plates). This formula is derived from the Growth Inhibition calculation used in the National Cancer Institute's NCI-60 high throughput screen. For the purposes of this disclosure, all data analysis was performed in Growth Inhibition (except where noted).

A GI reading of 0% represents no growth inhibition and would occur in instances where the T reading at 3 days is comparable to the V reading at the respective time-period. A GI of 100% represents complete growth inhibition (cytostasis) and in this case cells treated with compound for 3 days would have the same endpoint reading as TO control cells. A GI of 200% represents complete death (cytotoxicity) of all cells in the culture well and in this case the T reading at 3 days will be lower than the TO control (values near or at zero). See FIG. 4 below.

FIG. 4. Dose Response curve representations of cytostatic vs cytotoxic compound activity.

Inhibition is also provided as a measure of cell viability. Inhibition levels of 0% represent no inhibition of cell growth by treatment. Inhibition of 100% represents no doubling of cell numbers during the treatment window. Both cytostatic and cytotoxic treatments can yield an Inhibition percentage of 100%. Inhibition percentage is calculated as the following:

I=1−T/U
where T is the treated and U is the untreated/vehicle control.

Single Agent Curve fitting

The Horizon Chalice Analyzer software allows two ways of visualizing single agent dose response curves. The Logistics Curve fit modeling in Analyzer uses sigmoidal modeling of the data points. In most contexts, the Logistics Curve fit will accurately model the dose response curve. Caution should be used in cases of poor modeling, which can be observed with either steep dose response curves or shallow dose response curves that have a plateau in activity across a wide dose range. Poor modeling can result in inaccurate reporting of IC/GI values which, in turn, could lead to poor Loewe Additivity combination modeling and artificially high Synergy Scores for combination data.

Combination Effect Reference Models

Combination effects can be most readily characterized by comparing each data point to that of a combination reference model that was derived from single agent curves (refer to single agent curve fitting section below). Three models are generally used: (1) the Highest Single Agent is a simple reference model where the expected combination effect is the maximum of the single agent responses at corresponding concentrations; (2) the Bliss Independence model represents the statistical expectation for independent competing inhibitors; and (3) the Loewe Additivity model represents the expected response if both agents are actually the same compound and is the most generally accepted reference for synergy. Both HSA and Bliss are easily calculated, however determining Loewe Additivity is experimentally demanding and requires well-sampled single agent dose curves, data interpolation and iterative root finding for computation of the Loewe additive response surface.

Highest Single Agent Model

The Highest Single Agent (HSA) model is based simply on the intuition that if a combination's effect exceeds the effect level of each of its constituents, there must be some combination interaction. This model is generally selected for the analysis of combinations where there is a limited dose range or ratio available. Mathematically, the HSA model describes simple superposition of the single agent curves:

I_{HSA}(C_{X},C_{Y})=max(I_{X},I_{Y})

where C_{X,Y} are the concentrations of the X and Y compound, and I_{X} and I_{Y} are inhibitions of the single agents at C_{X,Y}. It is also useful to calculate a volume score (HSA Volume) between the data and the HSA surface to characterise the overall strength of combination effects.

Empirically derived combination matrices are compared to their respective HSA additivity models constructed from experimentally collected single agent dose response curves. Summation of this excess additivity across the dose response matrix is referred to HSA Volume. Positive HSA Volume suggests potential synergy, while negative HSA Volume suggests potential antagonism.

It is important to note that the HSA model is a simple reference model where the expected combination effect is the maximum of the single agent response at corresponding concentrations. Hence, experimental noise of the single agent activity (typically observed below GI 30-50%) can contribute to artificially high HSA Volume Score thereby, adversely affecting the accurate assessment of combination activates. Therefore, upon establishing an HSA Volume threshold, data was manually curated to verify combination interactions observed in the dose matrix.

As other parameters are available for further investigation in the Chalice software, the other combination effect reference models are briefly discussed below.

Bliss Independence Model

The Bliss Independence Model corresponds to multiplicative probabilities for effects in growth measures and is the preferred reference for synergy in some contexts, especially genetics. Bliss boosting, which models boosts in efficacy at high concentrations different from what the single agents can achieve, is adapted from the Bliss independence model that corresponds to a multiplicative effect in growth measures. The Bliss calculation is:

I_{Bliss}(C_{X},C_{Y})=I_{X}+I_{Y}−I_{X}I_{Y}
where C_{X,Y} are the concentrations of the X and Y compound, and I_{X} and I_{Y} are inhibitions of the single agents at C_{X,Y}. The Bliss volume score represents the statistical expectation for independent competing inhibitors. Bliss Independence has often been favoured because it can be directly calculated from minimally sampled experiments without single agent response curve interpolation or iterative root finding.

Loewe Additivity Model

The Loewe additivity model is dose-based and applies only to the activity levels achieved by the single agents. Loewe Volume is used to assess the overall magnitude of the combination interaction in excess of the Loewe additivity model. Loewe Volume is particularly useful when distinguishing synergistic increases in a phenotypic activity (positive Loewe Volume) versus synergistic antagonisms (negative Loewe Volume). When antagonisms are observed, the Loewe Volume should be assessed to examine if there is any correlation between antagonism and a particular drug target-activity or cellular genotype. This model defines additivity as a non-synergistic combination interaction where the combination dose matrix surface should be indistinguishable from either drug crossed with itself. The calculation for Loewe additivity is:

I_{Loewe} that satisfies (X/X₁)+(Y/Y₁)=1
where X₁ and Y₁ are the single agent effective concentrations for the observed combination effect I. For example, if 50% inhibition is achieved separately by 1 μM of drug A or 1 μM of drug B, a combination of 0.5 μM of A and 0.5 μM of B should also inhibit by 50%.

For the present analysis, as seen in the example below for the combinations of enhancee with enhancer, empirically derived combination matrices were compared to their respective Loewe additivity models constructed from experimentally collected single agent dose response curves. Summation of this excess additivity across the dose response matrix is referred to as Loewe Volume. Positive Loewe volume suggests potential synergy, whilst negative Loewe volume suggests potential antagonism.

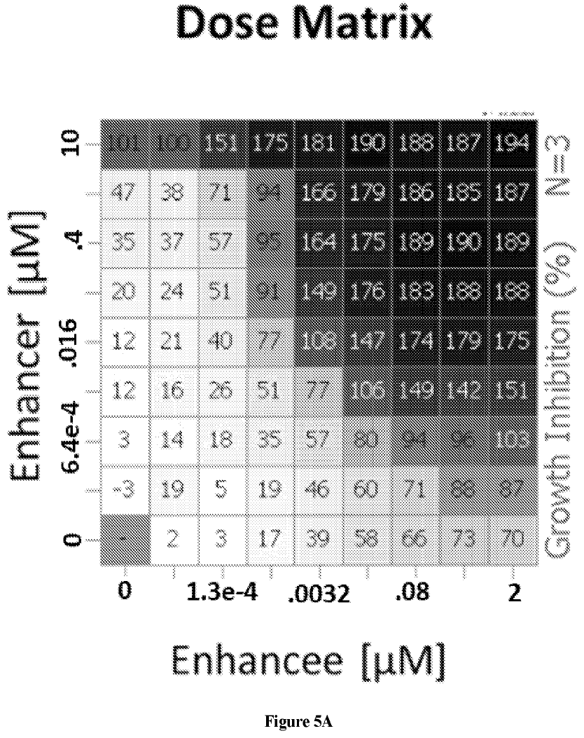

FIG. 5A: Dose Matrix; FIG. 5B: Loewe Model; FIG. 5C: Loewe Excess.

Synergy Score Analysis

To measure combination effects in excess of Loewe additivity, Horizon has devised a scalar measure to characterize the strength of synergistic interaction termed the Synergy Score. The Synergy score is calculated as:

Synergy Score=log f_{X} log f_{Y}Σmax(0,I_{data})(I_{data}−I_{Loewe})

The fractional inhibition for each component agent and combination point in the matrix is calculated relative to the median of all untreated/vehicle-treated control wells. The Synergy Score equation integrates the experimentally-observed activity volume at each point in the matrix in excess of a model surface numerically derived from the activity of the component agents using the Loewe model for additivity. Additional terms in the Synergy Score equation (above) are used to normalize for various dilution factors used for individual agents and to allow for comparison of synergy scores across an entire experiment. The inclusion of positive inhibition gating or an I_{data} multiplier removes noise near the zero-effect level, and biases results for synergistic interactions at that occur at high activity levels. Combinations with higher maximum Growth Inhibition (GI) effects or those which are synergistic at low concentrations will have higher Synergy Scores.

Potency shifting was evaluated using an isobologram, which demonstrates how much less drug is required in combination to achieve a desired effect level, when compared to the single agent doses needed to reach that effect. The isobologram was drawn by identifying the locus of concentrations that correspond to crossing the indicated inhibition level. This is done by finding the crossing point for each single agent concentration in a dose matrix across the concentrations of the other single agent. Practically, each vertical concentration C_{Y} is held fixed while a bisection algorithm is used to identify the horizontal concentration C_{X} in combination with that vertical dose that gives the chosen effect level in the response surface Z(C_{X},C_{Y}). These concentrations are then connected by linear interpolation to generate the isobologram display. For synergistic interactions, the isobologram contour falls below the additivity threshold and approaches the origin, and an antagonistic interaction would lie above the additivity threshold. The error bars represent the uncertainty arising from the individual data points used to generate the isobologram. The uncertainty for each crossing point is estimated from the response errors using bisection to find the concentrations where Z−σ_{Z}(C_{X},C_{Y}) and Z+σ_{Z}(C_{X},C_{Y}) cross I_{cut}, where σ_{Z} is the standard deviation of the residual error on the effect scale.

Combination Index

Potency shifting can also be scored using combination index (CI). For a chosen iso-effect level (I_{cut}), CI is calculated as:

CI=(C_{X}/EC_{X})+(C_{Y}/EC_{Y})
where (C_{X}/EC_{X}) for a particular data point is the ratio of the X compound's measured concentration to its effective concentration at the chosen effect level. The CI is a rough estimate of how much drug was needed in combination relative to the single agent doses required to achieve the chosen effect level. CI values in the range of 0.5-0.7 are typical for in vitro measurements of current clinical combinations. The CI error (σCI) is calculated using standard error propagation through the CI calculation based on the isobologram errors. In the Chalice Analyzer, the Best CI is reported from the many CI values calculated for each I_{cut} crossing combination. Among all the measured CI values, the one with the largest single-to-noise level (1−CI)/σCI is reported as the Best CI. Best CI Level is the effect level from which the CI is calculated.

Cell Line Panel

| | | | | | Seeding |
|---|---|---|---|---|---|
| | | KRAS | | Doubling | Density |
| Cell Line | Tissue | Status | Media | Time (h) | (cpw) |
| AsPC-1 | Pancreas | G12D | RPMI with 10% FBS | 44 | 500 |
| Calu-1 | Lung | G12C | McCoy's 5A with 10% | 37 | 500 |
| | | | FBS | | |
| Gp2D | Colorectal | G12D | DMEM with 10% FBS | 36 | 500 |
| HCC1171 | Lung | G12C | RPMI with 10% FBS, | 59 | 500 |
| | | | 25 mM HEPES and 25 | | |
| | | | mM Sodium | | |
| | | | Bicarbonate | | |
| HCC44 | Lung | G12C | RPMI with 10% FBS | 32 | 500 |
| HPAF-II | Pancreas | G12D | EMEM with 10% FBS | 38 | 500 |
| KYSE-410 | Esophageal | G12C | RPMI with 10% FBS | 29 | 500 |
| LS-513 | Colorectal | G12D | RPMI with 15% FBS | 31 | 500 |
| MIA PaCa- | Pancreas | G12C | DMEM with 10% FBS | 29 | 500 |
| 2 | | | and 2.5% horse serum | | |
| NCI-H1373 | Lung | G12C | RPMI with 10% FBS | 39 | 500 |
| NCI-H1792 | Lung | G12C | RPMI with 10% FBS | 32 | 500 |
| NCI-H2030 | Lung | G12C | RPMI with 10% FBS | 30 | 500 |
| NCI-H23 | Lung | G12C | RPMI with 10% FBS | 34 | 500 |
| NCI-H358 | Lung | G12C | RPMI with 10% FBS | 36 | 500 |
| Panc 04.03 | Pancreas | G12D | RPMI with 15% FBS | 37 | 500 |
| | | | and 10 Units/ml human | | |
| | | | insulin | | |
| SW48 | Colorectal | WT | RPMI with 10% FBS | 31 | 500 |
| SW48 | Colorectal | G12C | RPMI with 10% FBS | 30 | 500 |
| KRAS | | | | | |
| (G12C/+) | | | | | |
| SW48 | Colorectal | G12D | RPMI with 10% FBS | 37 | 500 |
| KRAS | | | | | |
| (G12D/+) | | | | | |
| SW837 | Colorectal | G12C | RPMI with 10% FBS | 43 | 500 |
| TOV-21G | Ovary | G13C | MCDB 105:MEDIUM | 38 | 500 |
| | | | 199 (1:1) with 15% FBS | | |
| | | | and 1.5 g/L Sodium | | |
| | | | Bicarbonate | | |

Compound Panel

| | | | Starting | |
|---|---|---|---|---|
| | | Chalice | Concentration | |
| Enhancee | Alias | ID | [μM] | Fold Dilution |
| Voruciclib | MEI-522 | C-22087 | 5 | 3 |

| | | Starting | |
|---|---|---|---|
| | Chalice | Concentration | |
| Enhancer | ID | [μM] | Fold Dilution |
| AMG-510 | C- | 3 | 3 |
| | 22440 | | |
| MTRX-849 | C- | 3 | 3 |
| | 23068 | | |
| Volasertib | C20609 | 1 | 3 |
| Onvansertib | C- | 1 | 3 |
| | 23192 | | |

Compound Panel - cell line specific combinations

| | | Number of | | | | |
|---|---|---|---|---|---|---|
| Cell Line | Tissue | Combinations | Enhancers combined with Voruciclib | | | |
| AsPC-1 | Pancreas | 4 | AMG510 | MRTX849 | Onvansertib | Volasertib |
| Calu-1 | Lung | 2 | AMG510 | MRTX849 | | |
| Gp2D | Colorectal | 4 | AMG510 | MRTX849 | Onvansertib | Volasertib |
| HCC1171 | Lung | 4 | AMG510 | MRTX849 | Onvansertib | Volasertib |
| HCC44 | Lung | 4 | AMG510 | MRTX849 | Onvansertib | Volasertib |
| HPAF-II | Pancreas | 4 | AMG510 | MRTX849 | Onvansertib | Volasertib |
| KYSE-410 | Esophageal | 4 | AMG510 | MRTX849 | Onvansertib | Volasertib |
| LS-513 | Colorectal | 4 | AMG510 | MRTX849 | Onvansertib | Volasertib |
| MIA PaCa-2 | Pancreas | 2 | AMG510 | MRTX849 | | |
| NCl-H1373 | Lung | 2 | AMG510 | MRTX849 | | |
| NCl-H1792 | Lung | 2 | AMG510 | MRTX849 | | |
| NCl-H2030 | Lung | 2 | AMG510 | MRTX849 | | |
| NCl-H23 | Lung | 2 | AMG510 | MRTX849 | | |
| NCl-H358 | Lung | 4 | AMG510 | MRTX849 | Onvansertib | Volasertib |
| Panc 04.03 | Pancreas | 4 | AMG510 | MRTX849 | Onvansertib | Volasertib |
| SW48 | Colorectal | 4 | AMG510 | MRTX849 | Onvansertib | Volasertib |
| SW48 | Colorectal | 4 | AMG510 | MRTX849 | Onvansertib | Volasertib |
| KRAS | | | | | | |
| (G12C/+) | | | | | | |
| SW48 | Colorectal | 4 | AMG510 | MRTX849 | Onvansertib | Volasertib |
| KRAS | | | | | | |
| (G12D/+) | | | | | | |
| SW837 | Colorectal | 4 | AMG510 | MRTX849 | Onvansertib | Volasertib |
| TOV-21G | Ovary | 4 | AMG510 | MRTX849 | Onvansertib | Volasertib |

### Example 5: Combining Voruciclib with Inhibitors of KRAS Pathway

KRAS inhibitors:

| | | Generic | | | |
|---|---|---|---|---|---|
| Drug Target | Drug Name | Name | Company Name | Indication | Development Stage |
| KRAS G12C inhibitor | sotorasib | sotorasib | Amgen Inc | NSCLC, CRC, solid | Pre-Registration, |
| | | [INN] | | tumor | Phase I, Phase II |
| | MRTX-849 | | Mirati Therapeutics Inc | NSCLC, CRC, PDAC, | Phase I, Phase II, |
| | | | | Ovarian, Endometrial, | Phase III |
| | | | | Bile Duct Cancer | |
| | D-1553 | | InventisBio Inc | Solid Tumor, NSCLC, | Phase I |
| | | | | CRC | |
| | GDC-6036 | | Genentech USA Inc | Solid Tumor, NSCLC, | Phase I |
| | | | | CRC | |
| | JNJ-74699157 | | Johnson & Johnson | Solid Tumor, NSCLC, | Phase I |
| | | | | CRC | |
| | ARS-1620 | | Araxes Pharma LLC | Lung Cancer | Preclinical |
| | JAB-21000 | | Jacobio Pharma | NSCLC, CRC | Preclinical |
| | ATG-012 | | Antengene Corp Ltd | Solid Tumor | Preclinical |
| | RMC-6291 | | Revolution Medicines Inc | NSCLC, PDAC | Preclinical |
| | ARS-853 | | Araxes Pharma LLC | Oncology | Discovery |
| | SML-8731 | | Dana-Farber Cancer | Oncology | Discovery |
| | | | Institute Inc | | |
| KRAS (pan) inhibitor | BBP-454 | | TheRas Inc | Oncology | Preclinical |
| | BPI-001 | | BeyondSpring Inc | Oncology | Preclinical |
| KRAS G12D inhibitor | MRTX-1133 | | Mirati Therapeutics Inc | CRC, NSCLC, PDAC, | Preclinical |
| | | | | Endometrial | |
| KRAS mutant inhibitor | COTI-219 | | Cotinga Pharmaceuticals | SCLC, CRC | Preclinical |
| | | | Inc | | |
| SOS1:KRAS inhibitor | BI-1701963 | | Boehringer Ingelheim | NSCLC, CRC | Phase I, Preclinical |
| KRAS:PDE inhibitor | LYN-202 | | LynkCell Inc | Oncology | Discovery |
| Downregulates KRAS | MM-41 | | University College London | Pancreatic Cancer | Preclinical |
| and BC12 transcription. | | | | | |
| Inhibitor of KRAS | RMC-6236 | | Revolution Medicines Inc | CRC, NSCLC, | Preclinical |
| (G12D, G12V, G13C, | | | | pancreatic | |
| G13D, Q61H), NRAS | | | | | |
| (Q61X), and HRAS | | | | | |
| mutants. | | | | | |

TKI + RAF inhibitor:

| Drug | | | Brand | | | Development |
|---|---|---|---|---|---|---|
| target | Drug Name | Generic Name | Name | Company Name | Indication | Stage |
| RTK and | regorafenib | regorafenib | Stivarga, | Bayer | GIST, CRC, HCC | Marketed, Phase |
| RAF | | | Nublexa | | | I, Phase II, Phase |
| inhibitor | | | | | | III |
| | regorafenib | regorafenib | Regonat | Natco Pharma Ltd | GIST, HCC, CRC | Marketed |
| | sorafenib tosylate | sorafenib tosylate | Nexavar | Bayer | Thyroid, HCC, RCC | Marketed, Phase |
| | | | | | | II, Phase III |
| | sorafenib tosylate | sorafenib tosylate | Sorafenib | G.L. Pharma | HCC, RCC | Marketed |
| | | | Tosylate | GmbH | | |
| | sorafenib tosylate | sorafenib tosylate | Sorafenib | Mylan Pharma, | HCC, RCC | Marketed |
| | | | Tosylate, | Natco Pharma Ltd | | |
| | | | Sorafenat | | | |
| | sorafenib tosylate | sorafenib tosylate | Sorafenib | Sandoz, Hexal | RCC, HCC | Marketed |
| | | | Tosylate | | | |
| | sorafenib tosylate | sorafenib tosylate | Sorafenib | Australian Primary | HCC, RCC | Marketed |
| | | | Tosylate | Hemp Ltd | | |
| | sorafenib tosylate | sorafenib tosylate | Sorafenib | Zentiva | HCC, RCC | Marketed |
| | | | Tosylate | | | |
| | sorafenib tosylate | sorafenib tosylate | Sorafenib | Alvogen Inc | HCC, RCC | Marketed |
| | sorafenib tosylate | sorafenib tosylate | Sorafenib | Teva | HCC, RCC | Marketed |
| | | | Tosylate | Biopharmaceuticals | | |
| | sorafenib tosylate | sorafenib tosylate | Sorafenib | Medac Pharma Srl | HCC, RCC, thyroid | Marketed |
| | | | Tosylate | | | |
| | sorafenib tosylate | sorafenib tosylate | Sorafenib | Nativa | Thyroid Cancer, HCC, | Marketed |
| | | | | | RCC | |
| | sorafenib tosylate | sorafenib tosylate | Sorafenib | EG Labo | HCC, RCC | Marketed |
| | | | Tosylate | EuroGenerics, | | |
| | | | | STADA | | |
| | sorafenib tosylate | sorafenib tosylate | Soranib | Cipla Ltd | Thyroid Cancer, HCC, | Marketed |
| | | | | | RCC | |
| | sorafenib tosylate | sorafenib tosylate | Sorafenib | PharOs Ltd | HCC, RCC | Marketed |
| | | | Tosylate | | | |
| RTK and | donafenib | | Zepsun | Suzhou Zelgen | CRC, HCC, thyroid, | Phase I, Phase II, |
| RAF | | | | Biopharmaceutical | esophageal, PDAC, | Phase III |
| inhibitor | | | | Co | nasopharyngeal, bile | |
| RTK and | | | | | duct, AML, head and | |
| RAF | | | | | neck, SCC | |
| inhibitor | (MG-005 + | sorafenib | | Metagone Biotech | RCC, liver, lung | Phase II |
| | sorafenib) | | | Inc | | |
| | (MG-010 + | sorafenib | | Metagone Biotech | NSCLC, RCC, HCC, | Phase II, |
| | sorafenib) | | | Inc | CRC, gastric, solid | Preclinial |
| | | | | | tumor | |
| | hydroxychloroquine + | hydroxychloroquine | | VG Life Sciences | HCC | Phase II |
| | sorafenib tosylate | [INN] + sorafenib | | Inc | | |
| | | tosylate | | | | |
| | lifirafenib maleate | lifirafenib maleate | | BeiGene Ltd | Bile duct, NSCLC, | Phase II |
| | | | | | endometrial | |
| | RXDX-105 | | | F. Hoffmann-La | Ovarian, thyroid, solid | Phase I |
| | | | | Roche Ltd | tumor | |
| | SJP-1601 | | | Samjin Pharm Co | Colorectal Cancer | Preclinical |
| | | | | Ltd | | |
| RTK, RAF, | APL-102 | | | Apollomics Inc | NSCLC, esophageal, | IND/CTA Filed |
| MAPK | | | | | CRC, gastric, breast, | |
| inhibitor | | | | | HCC | |

RAF inhibitors:

| Drug | | | Brand | | | Development |
|---|---|---|---|---|---|---|
| Target | Drug Name | Generic Name | Name | Company Name | Indication | Stage |
| RAF | belvarafenib | belvarafenib | | Genentech, Hanmi | Solid Tumor, sarcomas, CRC, | Phase I |
| inhibitor | | [INN] | | Pharma | bladder, GIST, NSCLC, | |
| | | | | | melanoma | |
| | TAK-580 | | | Day One Biopharma, | Glioma | Phase I, |
| | | | | Takeda Pharma | | Preclinical |
| | DCBCI-0902 | | | Development Center | Melanoma | Preclinical |
| | | | | for Biotech | | |
| | INU-152 | | | Incheon National | CRC, melanoma | Preclinical |
| | | | | University | | |
| | REDX-05358 | | | Jazz Pharmaceuticals | Colorectal Cancer | Preclinical |
| | | | | Plc | | |
| | SJP-601 | | | Samjin Pharm Co | Oncology | Preclinical |
| | | | | Ltd | | |
| BRAF | encorafenib | encorafenib | Braftovi | Array BioPharma, | CRC, melanoma | Marketed, Phase |
| inhibitor | | | | Pierre Fabre, Ono | | II, Phase III |
| | | | | Pharma, Pfizer | | |
| BRAF | BGB-3245 | | | BeiGene Ltd, | Solid Tumor, NSCLC, CRC, | Phase I, Phase II |
| inhibitor | | | | MapKure | melanoma, PDAC | |
| | PLX-8394 | | | Fore Biotherapeutics | Astrocytoma, | Phase II |
| | | | | Inc | Craniopharyngioma, GBM, Solid | |
| | | | | | Tumor | |
| | UB-941 | | | UBI Pharma Inc | Thyroid, melanoma, CRC, | IND/CTA Filed |
| | | | | | ovarian, lung | |
| | AFX-1251 | | | Afecta Pharma | Melanoma | Preclinical |
| | AZ-304 | | | AstraZeneca Plc | Colorectal Cancer | Preclinical |
| | KIN-002787 | | | Kinnate Biopharma | Melanoma, NSCLC, solid tumor | Preclinical |
| | XP-102 | | | Xynomic Pharma | NSCLC, CRC, hairy cell | Preclinical |
| | | | | | leukemia | |
| | LYN-204 | | | LynkCell Inc | Oncology | Discovery |
| BRAF | dabrafenib | dabrafenib | Tafinlar | Novartis | Thyroid, melanoma, NSCLC, | Marketed, Phase |
| V600E | mesylate | mesylate | | | glioma, lymphoma, astrocytoma, | II |
| inhibitor | | | | | GBM, MM, CRC | |
| | vemurafenib | vemurafenib | Zelboraf | Hoffmann-La Roche | Melanoma, Leukocyte Disorders | Marketed, Phase |
| | | | | Inc | (White Blood Cell Disorders), | I, Phase II |
| | | | | | NSCLC, CLL, GIST, NB, | |
| | | | | | ovarian, bladder, sarcomas, MM, | |
| | | | | | bile duct, thyroid, lymphoma, | |
| | | | | | prostate | |
| | ABM-1310 | | | ABM Therapeutics | Melanoma, thyroid, bile duct, | Phase I |
| | | | | Inc | GBM, CRC, ovarian, NSCLC | |
| | TQB-3233 | | | Jiangsu Chia-tai | Melanoma | Phase I |
| | | | | Tianging Pharma | | |
| BRAF | PF-07284890 | | | Pfizer Inc | Brain, melanoma, NSCLC, solid | Phase I |
| V600X | | | | | tumors | |
| inhibitor | | | | | | |

RAF + MEK inhibitors:

| | | | Brand | | | Development |
|---|---|---|---|---|---|---|
| Drug Target | Drug Name | Generic Name | Name | Company Name | Indication | Stage |
| RAF and | binimetinib + | binimetinib + | Braftovi + | Array BioPharma, | CRC, Melanoma, glioma, | Marketed, Phase I, |
| MEK | encorafenib | encorafenib | Mektovi | Pierre Fabre, Ono | MM, NSCLC, astrocytoma, | Phase II |
| inhibitor | | | | Pharma, Pfizer | PDAC | |
| RAF and | CKI-27 (VS- | | | Verastem Inc, Chugai | Ovarian, peritoneal, CRC, | Phase I, Phase II, |
| MEK 1 | 6766) | | | Pharmaceutical | endometrial, uveal | Preclinical |
| inhibitor | | | | | melanoma, NSCLC, | |
| | | | | | ovarian, MM, melanoma, | |
| | | | | | PDAC, prostate | |
| BRAF | dabrafenib | dabrafenib | | Novartis AG | Metastatic Colorectal | Phase II |
| V600E + | mesylate + | mesylate + | | | Cancer | |
| MEK 1 | panitumumab + | panitumumab + | | | | |
| inhibitor | trametinib | trametinib | | | | |
| | dimethyl | dimethyl | | | | |
| | sulfoxide | sulfoxide | | | | |
| FLT3, MEK1, | E-6201 | | | Eisai Co, Strategia | Melanoma, metastatic brain | Phase I |
| RAF1 | | | | Therapeutics | Tumor | |
| inhibitor | | | | | | |

MEK inhibitors:

| Drug | | | Brand | | | |
|---|---|---|---|---|---|---|
| Target | Drug Name | Generic Name | Name | Company Name | Indication | Development Stage |
| MEK 1 | EBI-1051 | | | Eternity Bioscience Inc | Melanoma, CRC | Preclinical |
| inhibitor | HL-085 | | | Binjiang Pharma, Inc. | Melanoma, CRC, | Phase I, Phase II, |
| | | | | | NSCLC, solid tumor | Preclinical |
| MEK 1/2 | cobimetinib | cobimetinib | Cotellic | Genentech, Roche | Melanoma, TNBC, | Marketed, Phase I, |
| inhibitor | fumarate | fumarate | | | urothelial tract, glioma, | Phase II |
| | | | | | MM, PDAC, AML, | |
| | | | | | RCC, NB, RMS, | |
| | | | | | neurofibrosarcoma, | |
| | | | | | solid tumors, fallopian | |
| | | | | | tube, peritoneal, | |
| | | | | | epithelial | |
| | binimetinib | binimetinib | Mektovi | Array BioPharma, | Melanoma, CRC, | Marketed, Phase I, |
| | | | | Pierre Fabre, Ono, | thyroid, NSCLC, | Phase II, Phase III |
| | | | | Pfizer | AML, TNBC, ALL, | |
| | | | | | bile duct, gallbladder, | |
| | | | | | GIST, glioma, biliary | |
| | | | | | tract | |
| MEK 1/2 | binimetinib + | binimetinib + | Braftovi + | Array BioPharma, | Melanoma, CRC, | Marketed, Phase I, |
| inhibitor | encorafenib | encorafenib | Mektovi | Pfizer, Pierre Fabre, | glioma, MM, NSCLC, | Phase II |
| | | | | Ono | astrocytoma, PDAC | |
| | selumetinib | selumetinib | Koselugo | AstraZeneca | ALL, glioma, NSCLC, | Phase I, Phase II, |
| | sulfate | sulfate | | | ovarian | Preclinical |
| | trametinib | trametinib | Mekinist | Novartis | NSCLC, melanoma, | Marketed, Phase I, |
| | dimethyl | dimethyl | | Pharmaceuticals | thyroid, TNBC, MM, | Phase II, Preclinical |
| | sulfoxide | sulfoxide | | | PDAC, bile duct, | |
| | | | | | endometrial, biliary | |
| | | | | | tract, glioma | |
| | mirdametinib | mirdametinib | | SpringWorks | CRC, NSCLC, PDAC, | Phase II |
| | | | | Therapeutics Inc | endometrial, | |
| | | | | | melanoma, ovarian, | |
| | | | | | thyroid | |
| | REC-4881 | | | Recursion Pharma | Solid Tumor, | Phase II |
| | | | | | Melanoma | |
| | trametinib | trametinib | | Novartis AG | MM, TNBC, | Phase I, Phase II |
| | dimethyl | dimethyl | | | endometrial cancer | |
| | sulfoxide + | sulfoxide + | | | | |
| | uprosertib | uprosertib | | | | |
| | CS-3006 | | | CStone Pharmaceuticals | Solid Tumor | Phase I |
| | | | | Co Ltd | | |
| | FCN-159 | | | Fochon Pharmaceutical | Melanoma, solid tumor | Phase I |
| | SHR-7390 | | | Jiangsu Hengrui | Solid Tumor, CRC, | Phase I |
| | | | | Medicine Co | lung, melanoma | |
| | TQB-3234 | | | Sino Biopharmaceutical | CRC, PDAC, thyroid, | Phase I |
| | | | | Ltd | melanoma | |
| | ABM-1383 | | | ABM Therapeutics Inc | Oncology | Preclinical |
| | CIP-137401 | | | AlloMek Therapeutics | Solid Tumor | Preclinical |
| | KZ-001 | | | Binjiang Pharma, Inc. | Lung, PDAC, | Preclinical |
| | | | | | melanoma | |
| | LNP-3794 | | | Lupin Ltd | Solid Tumor, NSCLC, | Preclinical |
| | | | | | CRC | |
| MEK 2 | ATI-450 | | | Aclaris Therapeutics | Breast, PDAC | Preclinical |
| inhibitor | | | | Inc | | |
| MEK 1/2 + | MK-2206 + | selumetinib | | Merck & Co Inc | NSCLC | Phase II |
| AKT | selumetinib | sulfate | | | | |
| inhibitor | sulfate | | | | | |
| MEK 1/2 + | durvalumab + | durvalumab + | | AstraZeneca Plc | CRC | Phase I |
| PDL1 | selumetinib | selumetinib | | | | |
| inhibitor | sulfate | sulfate | | | | |

ERK inhibitors:

| | | | | | Development |
|---|---|---|---|---|---|
| Drug Target | Drug Name | Generic Name | Company Name | Indication | Stage |
| ERK 1/2 inhibitor | ASTX-029 | | Astex Pharma | Solid Tumor | Phase I, Phase II |
| | LY-3214996 | | Eli Lilly and Co | NSCLC, PDAC, AML, | Phase I, Phase II |
| | | | | melanoma, CRC | |
| | NE-3107 | | Neurmedix Inc | Prostate Cancer | Phase II, Preclinical |
| | ulixertinib | ulixertinib [INN] | BioMed Valley | Melanoma, AML, CRC, | Phase I, Phase II |
| | | | Discoveries Inc | NSCLC, PDAC, Uveal | |
| | | | | Melanoma, Bile Duct | |
| | | | | Cancer | |
| | ASN-007 | | Asana BioSciences | CRC, PDAC, NSCLC, | Phase I |
| | | | LLC | Melanoma | |
| | ATG-017 | | Antengene Corp, | NSCLC, AML, MM, NHL, | Phase I, Preclinical |
| | | | Furen Pharma | Solid Tumor | |
| | BPI-27336 | | Betta | Melanoma, PDAC, Gastric, | Phase I |
| | | | Pharmaceuticals Co | CRC, lung, liver, solid | |
| | | | Ltd | tumors | |
| | MK-8353 | | Merck & Co Inc | CRC, rectal | Phase I |
| Inhibits ERK 2, Src, | JRP-890 | | Prous Institute for | Breast, lung cancer | Preclinical |
| Akt3, CDK2, COX- | | | Biomedical | | |
| 1, lipoxygenase. | | | Research SA | | |

### Example 6: Adagrasib and AMG-510 Combinations in Mia PaCa-2 Xenografts

Experiments performed using the CIVO platform: presagebio.com/civo-platform/. Voruciclib in combination with: Adagrasib (MRTX-849); a KRASG12C inhibitor from Mirati for NSCLC, CRC and other solid tumors. Currently in Phase 1/2b. Sotorasib (AMG-510); a KRASG12C inhibitor from Amgen for NSCLC. Currently in Phase 2 with breakthrough status.

Experimental Design

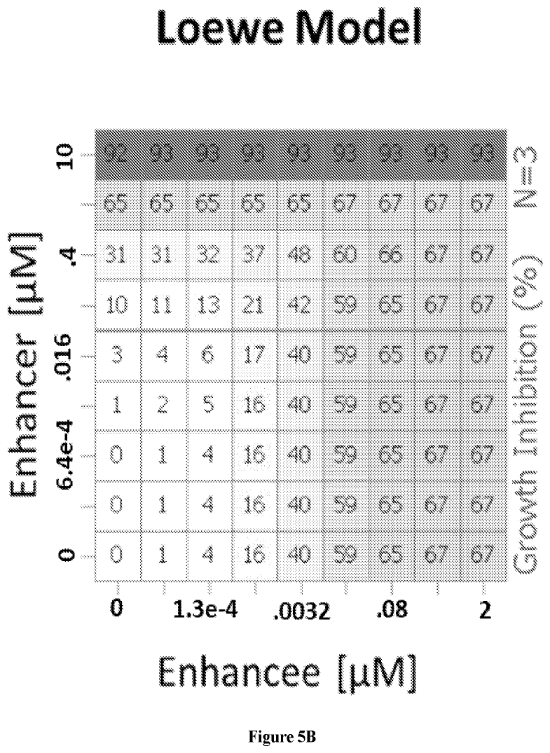

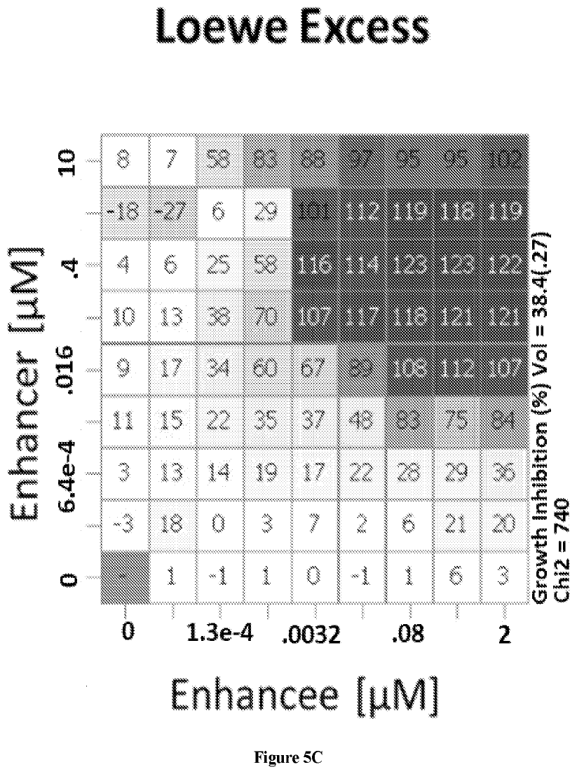

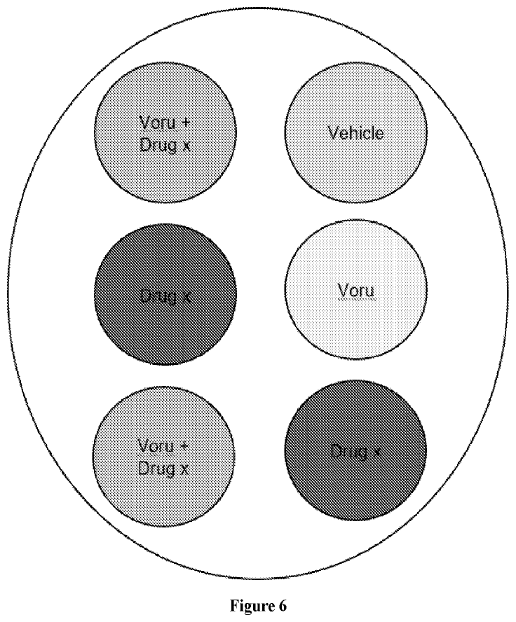

FIG. 6 illustrates an injection array; drug mass delivered: Voruciclib: 5 μg; AMG-510: 15 μg; Adagrasib: 15 μg. Replicate Tumors: n≥5. Time Point: 24 hr. Cell Line; MIA PaCa-2. Biomarker: CC3, H&E.

Investigation of Voruciclib+adagrasib combination in the Mia PaCa-2 pancreatic cancer model.

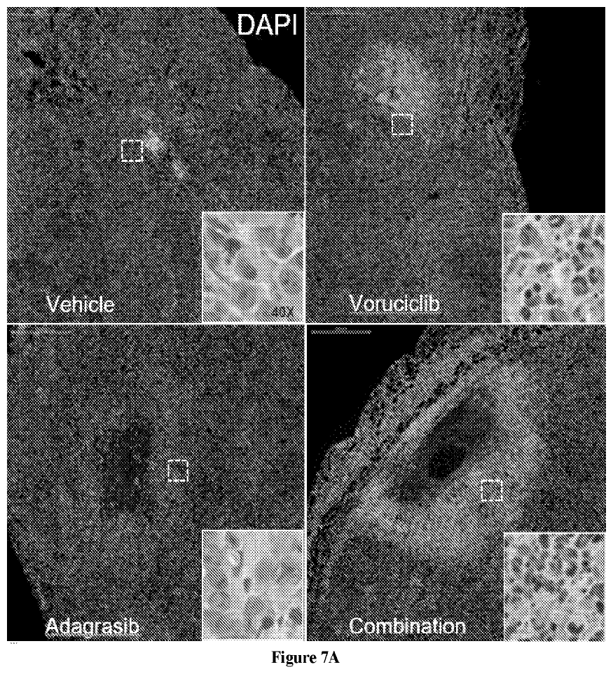

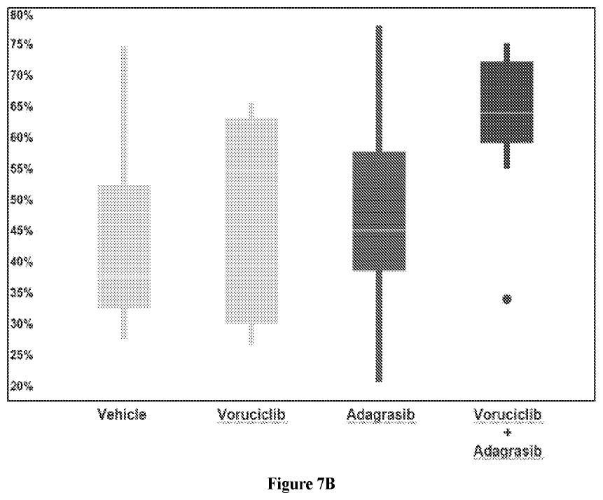

FIG. 7 illustrates that Combining Voruciclib and Adagrasib results in enhanced tumor cell death. FIG. 7A illustrates Cell death around each microinjection site measured by nuclear condensation and fragmentation. FIG. 7B illustrates % Cells with pyknotic/fragmented nuclei (dead cells). Data represents 5 tumors 2 Adagrasib injection sites and 1 Veh, Voru and Combo injection sites per tumor; 4 sections imaged per tumor.

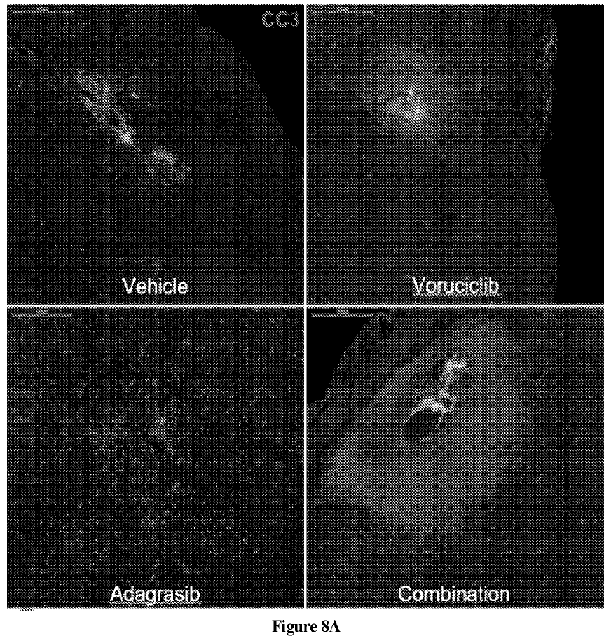

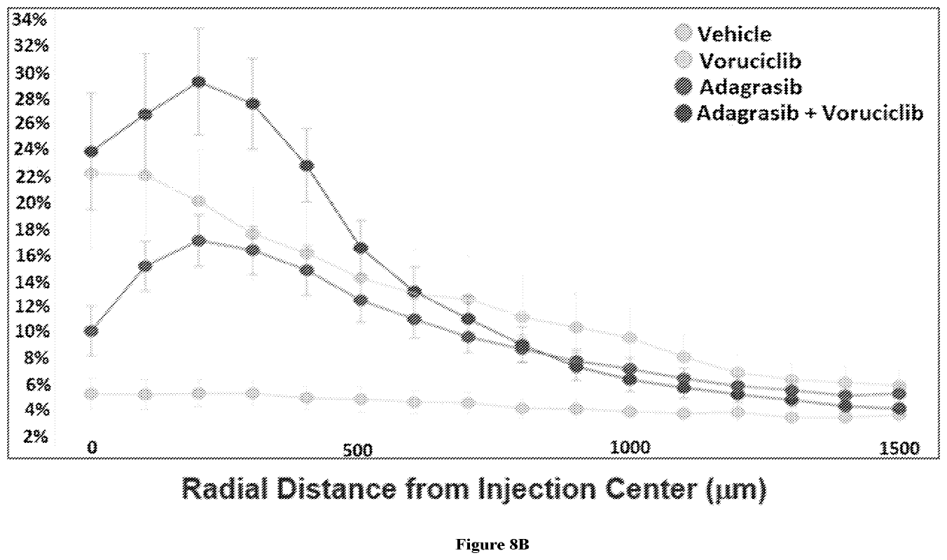

FIG. 8 illustrates that Combining Voruciclib and Adagrasib increases apoptotic cells. FIG. 8A apoptosis measured by CC3+ cells; apoptosis in combination site is increasing in radial zones where single agent apoptosis is falling. FIG. 8B illustrates % of Cell Area CC3+. Data represents 5 tumors 2 Adagrasib injection sites and 1 Veh, Voru and Combo injection sites per tumor; 4 sections imaged per tumor. Error Bars are Std Err Mean.

Investigation of Voruciclib+AMG-510 combination in the Mia PaCa-2 pancreatic cancer model.

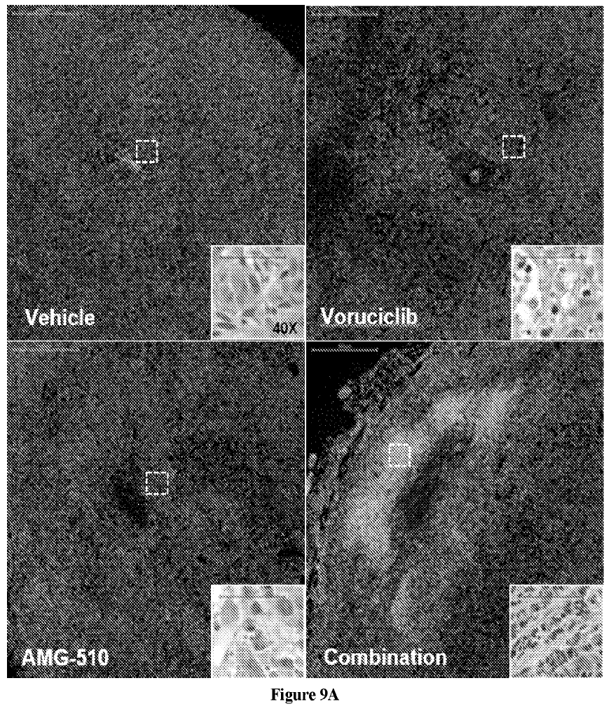

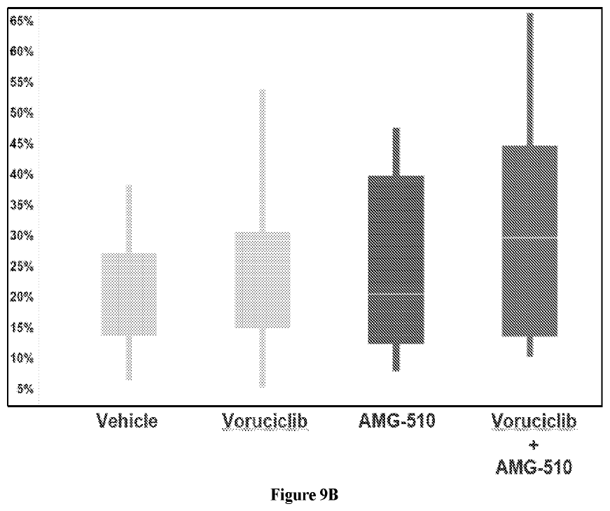

FIG. 9 illustrates that Combining Voruciclib and AMG-510 results in enhanced tumor cell death. FIG. 9A illustrates Cell death around each microinjection site measured by nuclear condensation and fragmentation. FIG. 9B illustrates % Cells with pyknotic/fragmented nuclei (dead cells). Data represents 5 tumors with duplicate combination and AMG-510 injection sites per tumor, single injection sites other conditions; 4 sections imaged per tumor.

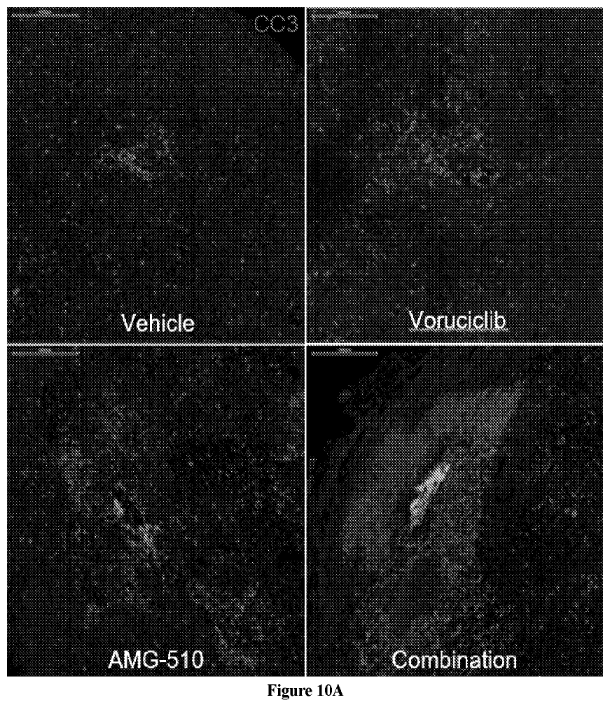

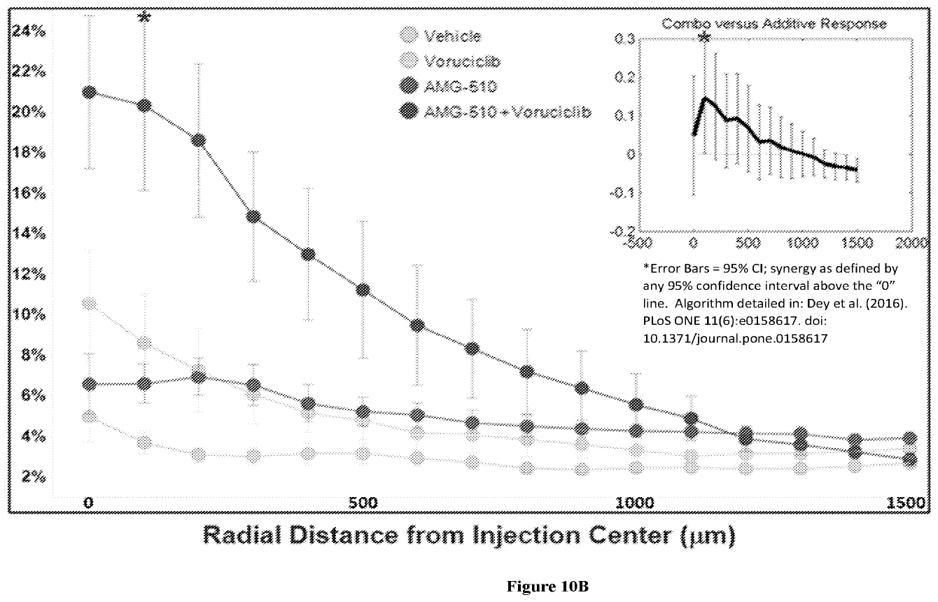

FIG. 10 illustrates that Combining Voruciclib and AMG-510 increases apoptotic cells. FIG. 10A illustrates apoptosis measured by CC3+ cells; combination induced apoptosis meets threshold for synergy, not just additivity. FIG. 10B illustrates % of Cell area CC3+. Data represents 5 tumors with duplicate combination and AMG-510 injection sites per tumor, single injection sites other conditions; 4 sections imaged per tumor. Error Bars=Stnd Err Mean.

### Example 7: CIVO Intratumoral Injection Experiments

Comparative In Vivo Oncology (CIVO) injects multiple drugs and/or drug combinations simultaneously in microdose quantities directly into a patient's tumor. The device delivers trackable drug “columns.” Co-injection with CIVO GLO enables injection site identification. Drugs interact with the tumor within the patient and then the tumor is surgically removed. The tumor is processed by sectioning cross-wise to the drug columns and placed onto slides for staining. Proprietary software and developed assays allow for deep profiling around each injection site to quantify and highlight immune profiles, cell signaling activations, tumor microenvironment (TME) impact, responder hypotheses, and/or drug combination potential.

CIVO was used to study the combination of voruciclib with each of adagrasib, sotorasib, onvansertib, and ME-344. Adagrasib (MRTX-849) is a KRAS^{G12C} inhibitor for NSCLC, CRC and other solid tumors that is currently in Phase 1/2b studies. Sotorasib (AMG-510) is a KRAS^{G12C} inhibitor for NSCLC that is currently in Phase 2 studies with breakthrough status. Onvansertib (NMS-P927) is a PLK1 inhibitor for multiple indications that is currently in Phase 1b studies for Myeloid Leukemia. ME-344 is a mitochondrial inhibitor for ovarian and small cell lung cancer that is currently in Phase 0 development.

FIG. 6 depicts the CIVO injection array. For the above studies, the time point is 24 hours, the cell line is MIA PaCA-2, and the biomarkers screened are cleaved caspase 3 (CC3) for apoptotic cell death and H&E. As a secondary biomarker, nuclear condensation and fragmentation captures drug-induced cell death that may not be sufficiently observed by CC3 alone. The results were obtained in ≥5 replicate tumors. 5 μg voruciclib, 15 μg AMG-510, 15 μg adagrasib, 3 μg onvansertib, and 15 μg ME-344 was delivered.

FIGS. 11A-11B depict data in HCC-44 NSCLC cell tumors demonstrating that the combination of voruciclib and adagrasib increases apoptosis. Elevated CC3 is observed at sites of the combination microinjection versus all controls. Microinjection of the combination resulted in significantly increased apoptotic (CC3+) cells vs sites exposed to either voruciclib or adagrasib alone. Several tumors exhibited large regions of necrosis, but combination effect at 24 h was observed despite this background. Further confirmation of combination effect via a conventional preclinical study with systemically administered drug is indicated.

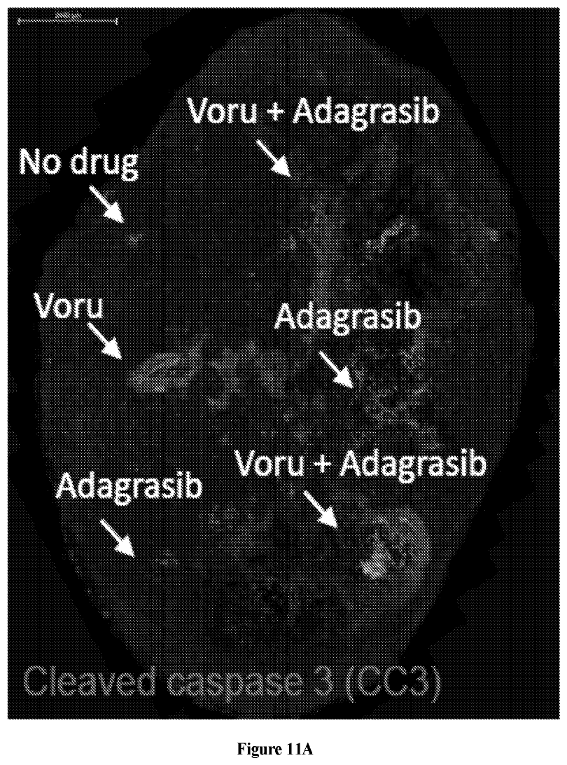

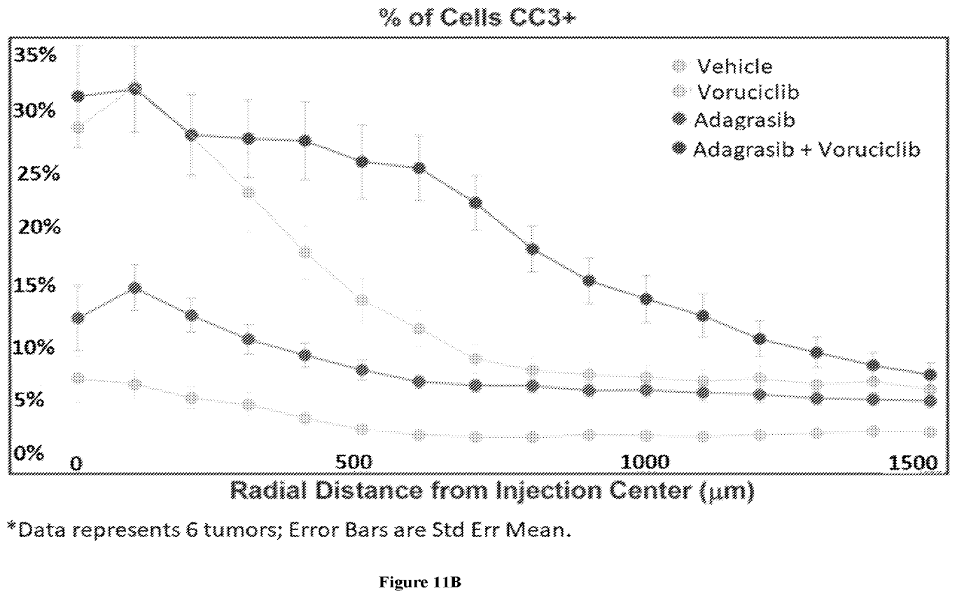

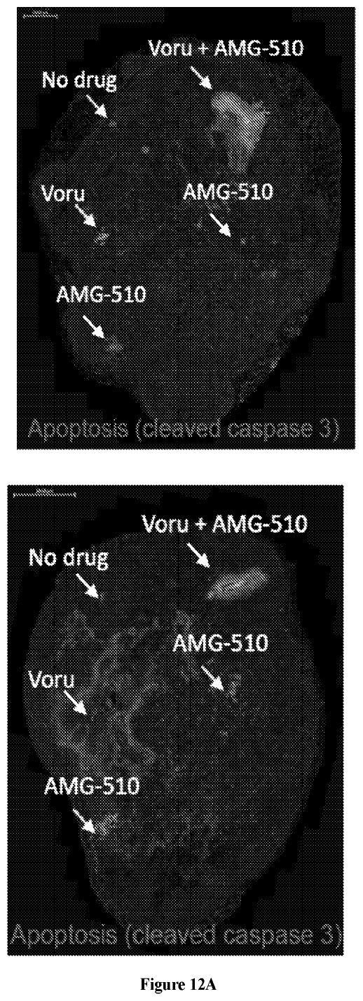

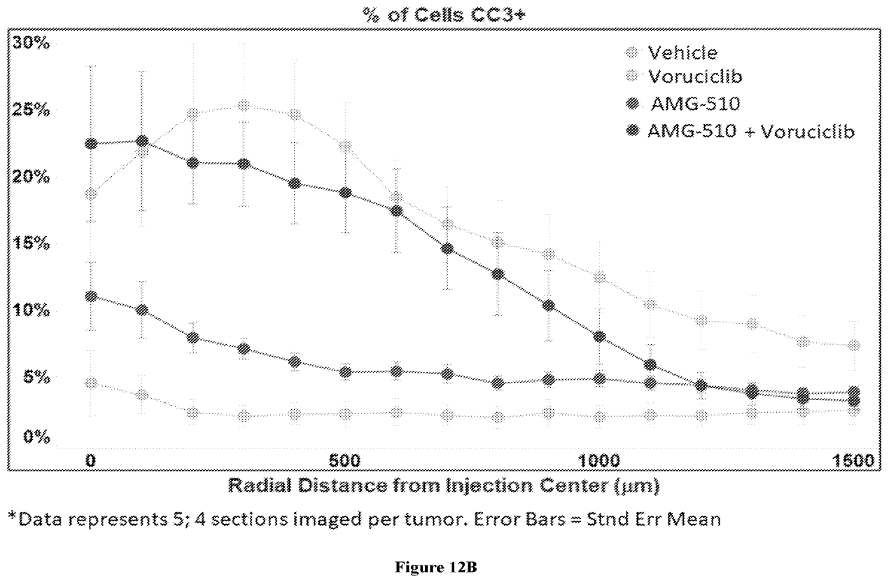

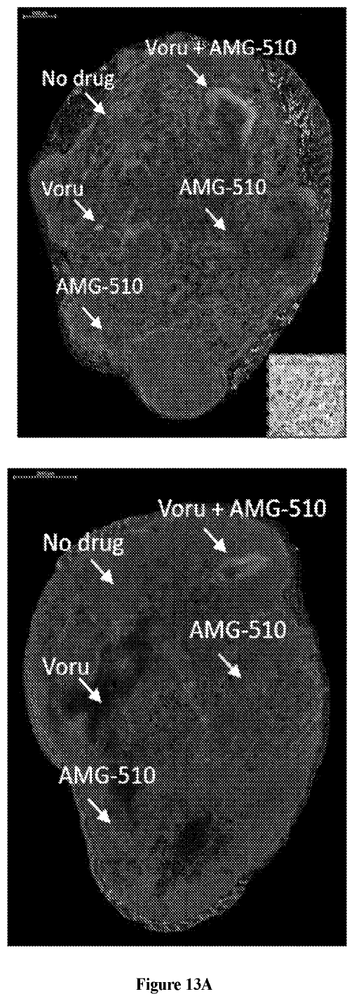

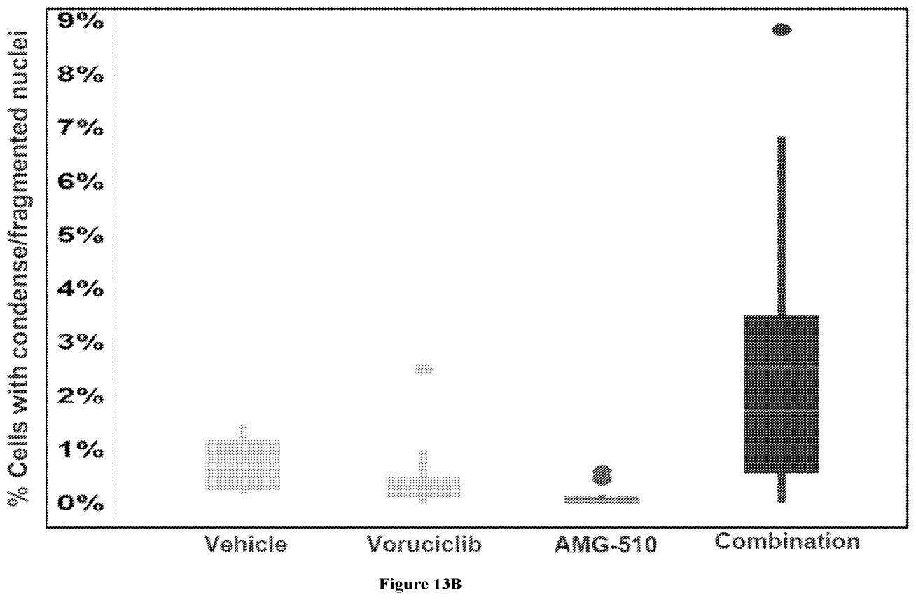

FIGS. 12A-12B depict data in HCC-44 xenografts demonstrating that the combination of voruciclib and AMG-510 increases cell death in some tumors. FIG. 12A depicts replicate tumors. The cell death observed is similar to what was observed in the adagrasib study, but this time, often coincided with sites of voruciclib injection. The injections will be re-attempted with a lower microdose of voruciclib to assess whether this effect was drug-related (vs coincidental to regions of stochastic necrosis). FIGS. 13A-13B depict morphological data in HCC-44 xenografts, which is easier to observe above the background. This data demonstrates that the combination of voruciclib and AMG-510 increases cell death. Similar to that observed with adagrasib, microinjection of the voruciclib and AMG-510 combination resulted in anti-tumor effects greater than those induced with either single agent. While evaluation of apoptotic cell death did not reveal a measurable elevation upon exposure to the combination compared to voruciclib alone (FIGS. 12A-12B), increased cell death observed by morphologies including condensed nuclear staining and nuclear fragmentation were observed (FIGS. 13A-13B).

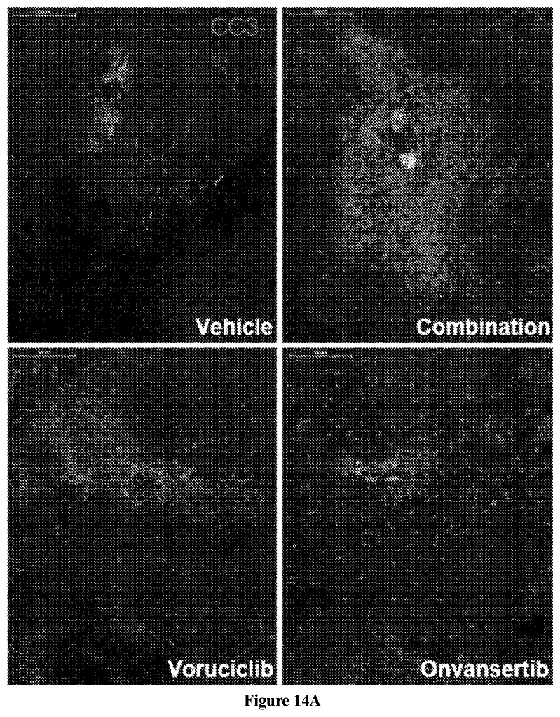

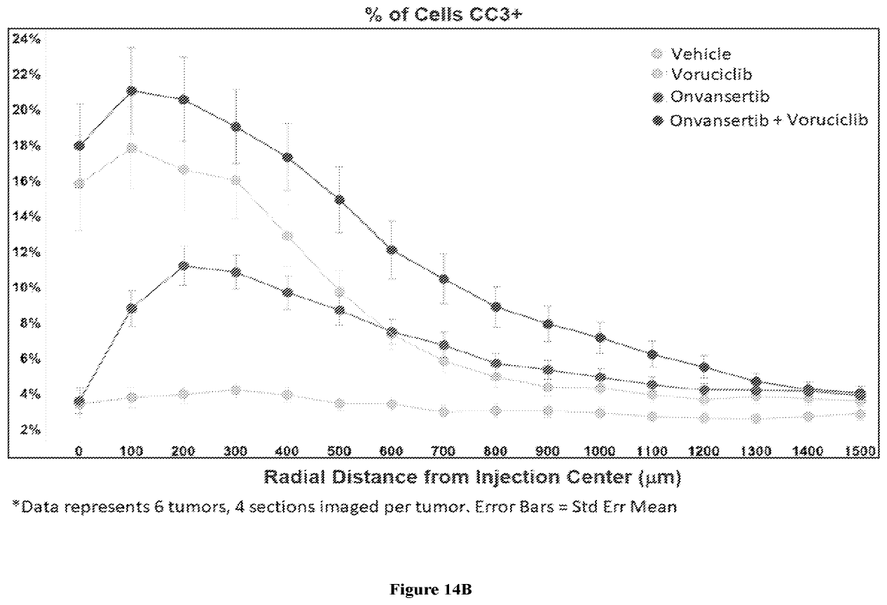

FIGS. 14A-14B depict data in MIA PaCa-2 xenografts demonstrating that the combination of voruciclib and onvansertib (a PLK1 inhibitor) increases apoptotic cells in a pancreatic cancer model. Elevated CC3 was observed at sites of the combination microinjection versus all controls and microinjection of the voruciclib and onvansertib combination resulted in greater apoptosis than either single agent. Voruciclib is likely the main driver of this effect and a lower voruciclib dose would likely result in a larger delta between single agent and combination induced apoptosis.

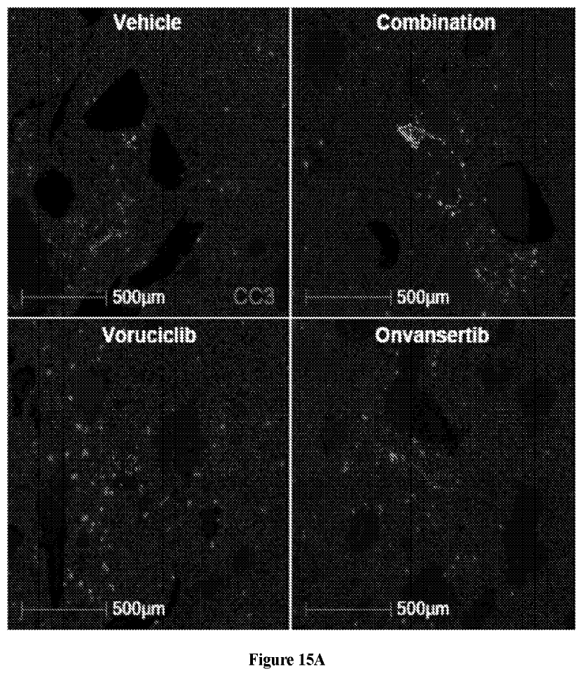

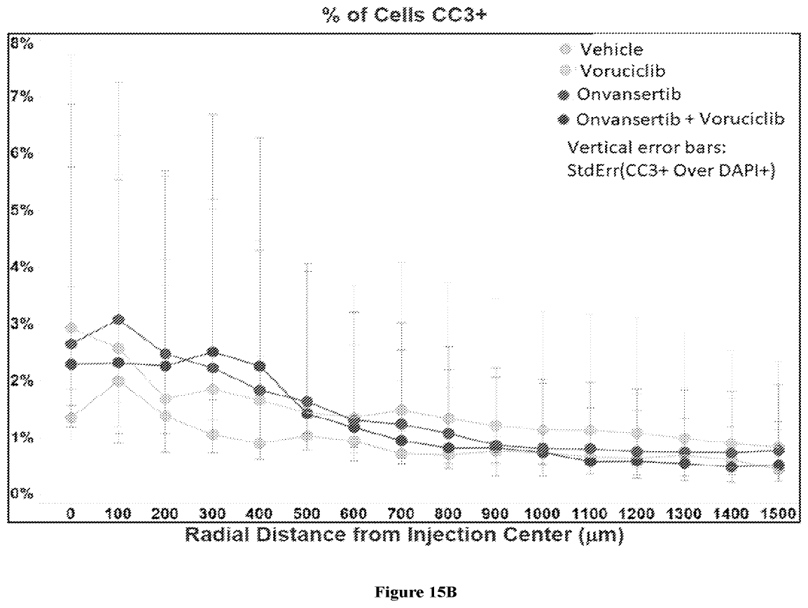

FIGS. 15A-15B depict data in Huh-7 xenografts demonstrating that the combination of voruciclib and onvansertib as well as the single agents do not result in an increase in apoptotic cells in a liver cancer model. CC3 is not elevated over background levels at any injection site.

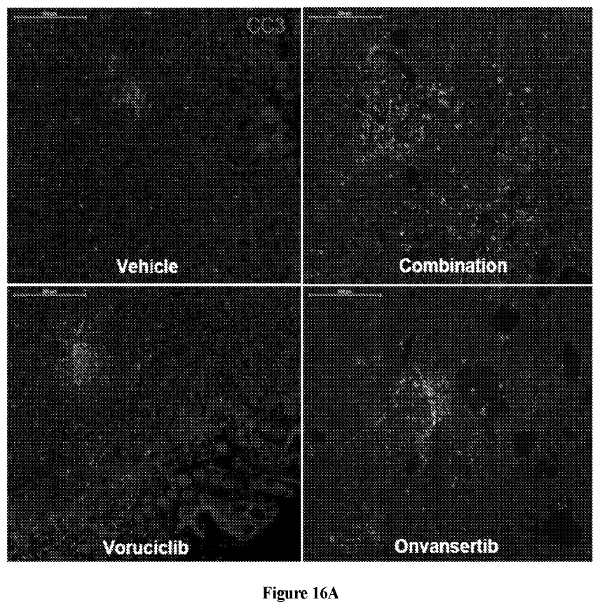

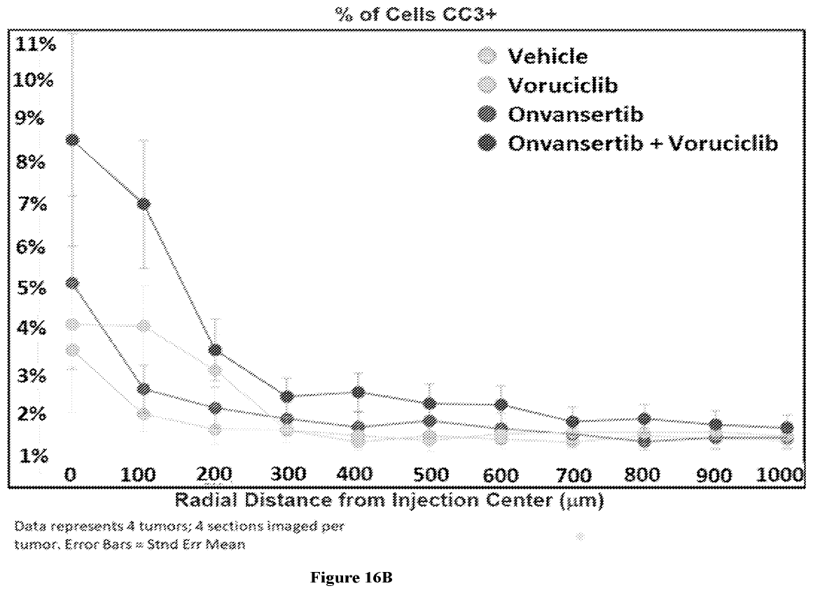

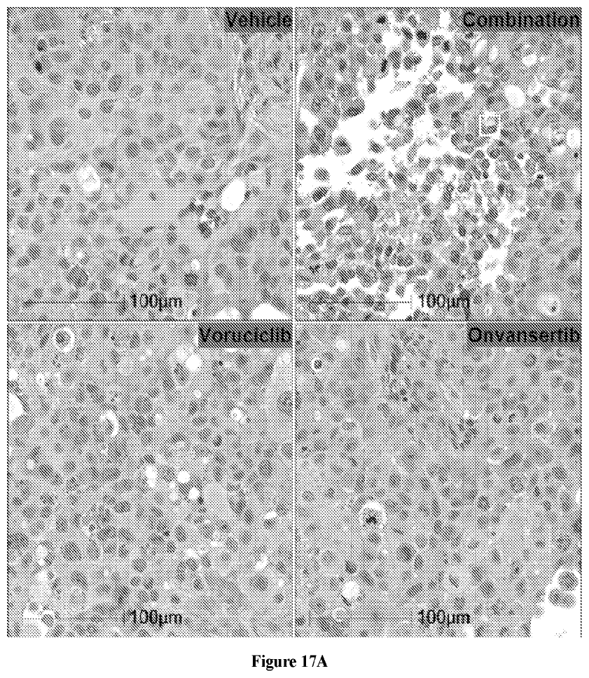

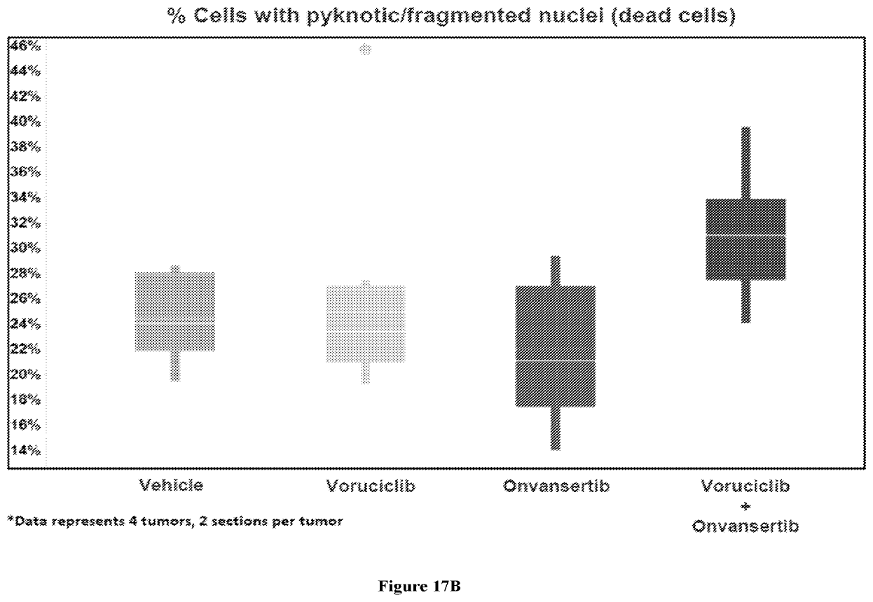

FIGS. 16A-16B depict data in an H441 lung cancer model ((NSCLC) KRAS G12V) demonstrating that combining voruciclib and onvansertib increases apoptotic cells. Slight elevated CC3 was observed at sites of the combination microinjection vs controls. FIGS. 17A-17B depict data in an H441 lung cancer model demonstrating that combining voruciclib and onvansertib results in enhanced tumor cell death. Therefore, the morphological phenotype matches the apoptotic staining results. These results show that microinjection of the combination increases apoptotic cells over either single agent, but only within zones of the highest drug concentration close to the injection site.

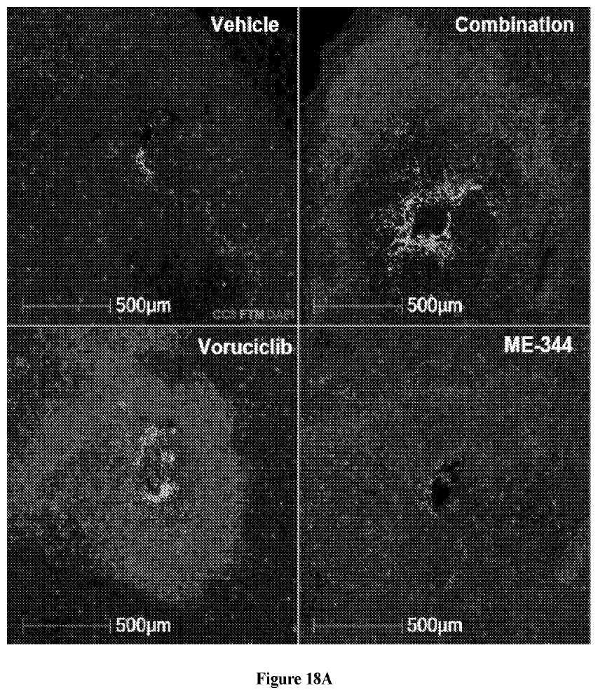

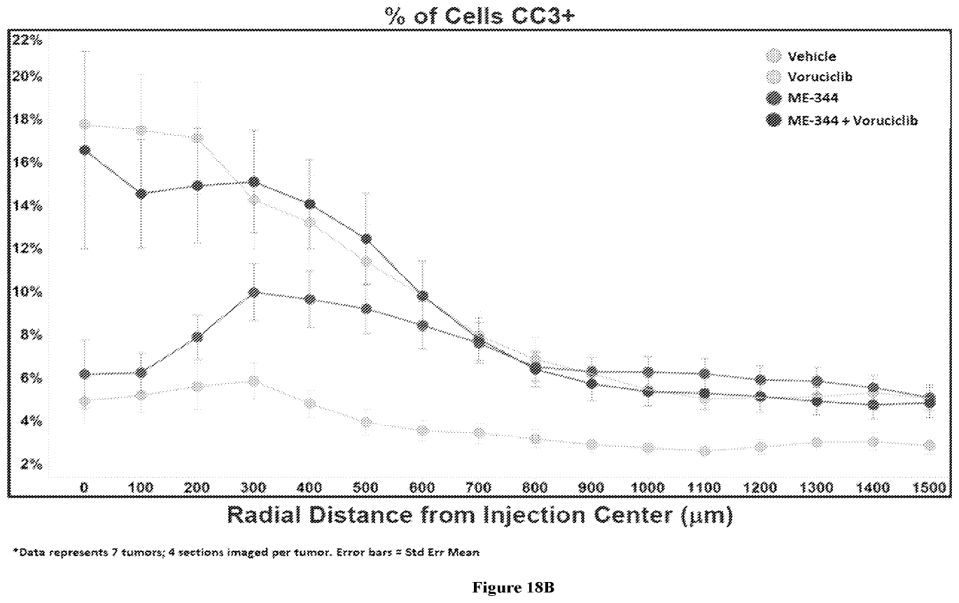

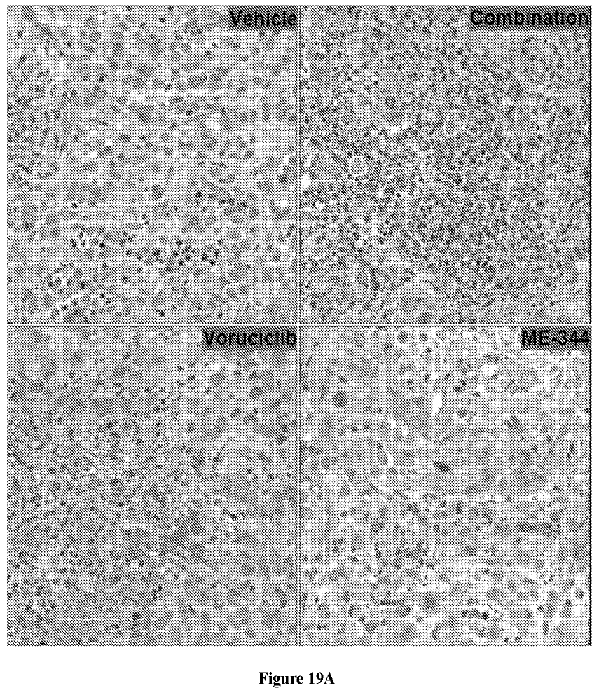

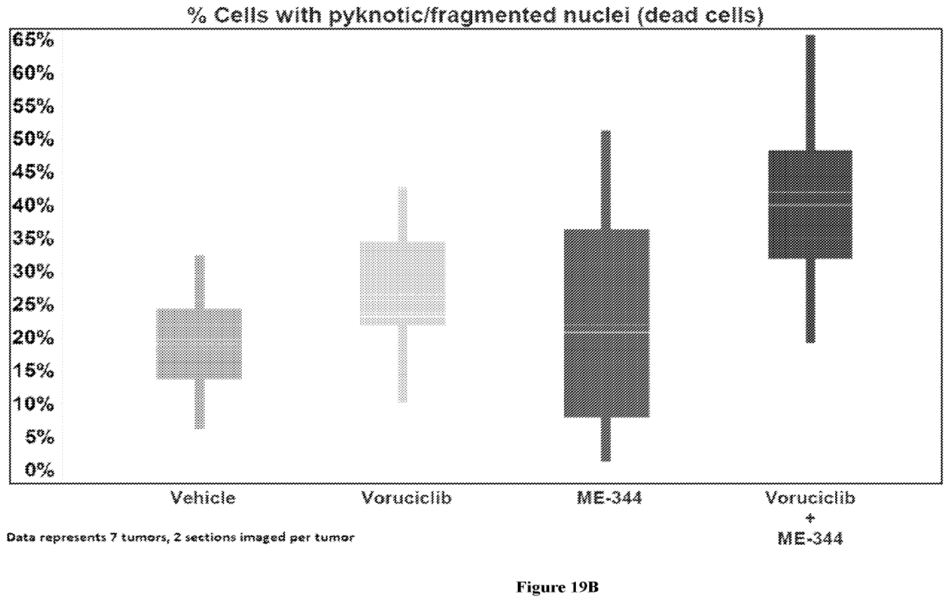

FIGS. 18A-18B and FIGS. 19A-19B provide the results of an investigation of voruciclib and ME-344 in MIA PaCa-2 xenografts. FIGS. 18A-18B demonstrate that the combination of voruciclib and ME-344 results in faster cell death than voruciclib alone, leading to larger area of post-apoptotic a-cellularity. Elevated CC3 was observed at sites of combination microinjection vs all controls. FIGS. 19A-19B demonstrate that combining voruciclib and ME-344 results in enhanced tumor cell death (note that the morphological phenotype was easier to observe above background).

Similar to the results with the AMG-510 combination, the data in FIGS. 18A-18B and 19A-19B demonstrate that the evaluation based on apoptotic cells death alone does not show an effect over voruciclib alone; however, this is likely due to large central areas of dead cells at the combination sites no longer staining positive for the cleaved caspase-3 marker. An investigation of the H&E staining reveals increased cell death observed by morphologies including condensed nuclear staining and nuclear fragmentation. Although not wishing to be limited by theory, it is believed that both an earlier time point and a lower dose of voruciclib would reveal a more dramatic combination effect over single agents.

FIG. 20 depicts results of the combination of voruciclib with adagrasib PaCa-2 KRAS G12C mutant PDAC tumors, wherein the combination was found to decrease MYC positive cells more than either single agent. The original paraffin embedded fixed tumor blocks were sliced and stained for MCL-1 and total MYC using antibodies specific to each. The MYC staining is in red. The MCL-1 staining is in yellow. The nuclei are stained in blue. For MYC and MCL-1, a decrease in the signal indicates an on-target effect of VOR.

FIG. 21 depicts cMYC and MCL-1 staining in MIA PaCa-2 PDAC tumors. The original paraffin embedded fixed tumor blocks were sliced and stained for MCL-1 and total MYC using antibodies specific to each. The MYC staining is in red. The MCL-1 staining is in yellow. The nuclei are stained in blue. For MYC and MCL-1, a decrease in the signal indicates an on-target effect of VOR.

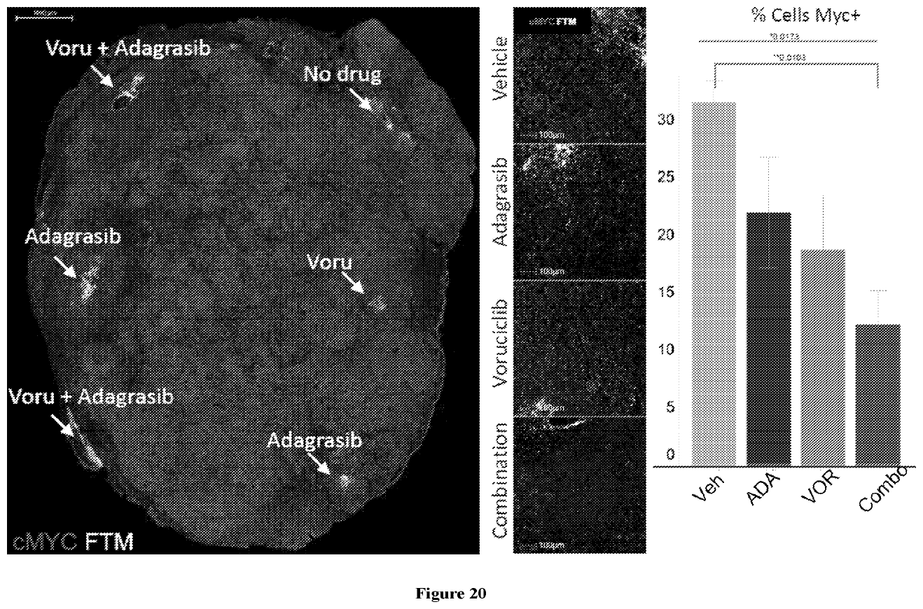

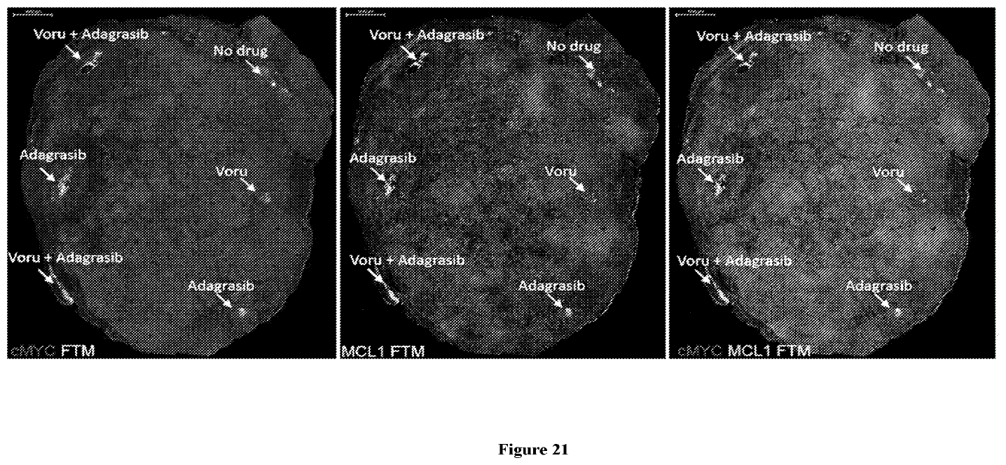

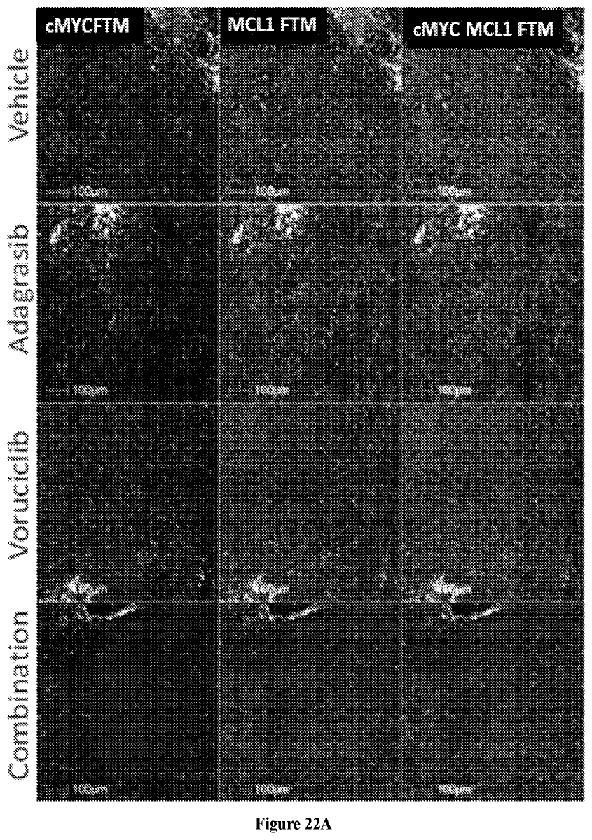

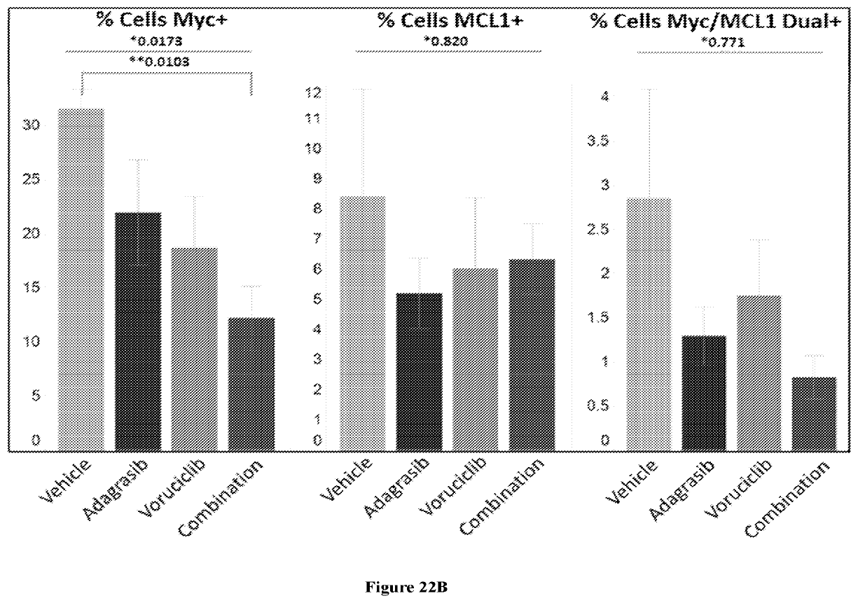

FIGS. 22A-22B provide data demonstrating that the combination injection of voruciclib and adagrasib in MIA Paca-2 PDAC tumors decreases MYC positive cells more than either single agent. No statistical difference in MCL-1 protein was observed from any condition due to the large variation at the vehicle site. In FIG. 22B, the quantification represents data from 8 tumors with 1000 μm diameter ROIs around injection sites, *ANOVA test for multiple groups, **pairwise p values are calculated from a Turkey HSD test.

The results in FIG. 20, FIG. 21, and FIGS. 22A-22B can be used to determine the effect of the combination of voruciclib and adagrasib on cMYC and MCL-1 regulation in MIA PaCa-2 cells. Microinjection of the combination resulted in a significantly decreased percentage of MYC positive cells. Although both single agents also resulted in a decrease in MYC, only the combination reached statistical significance. No statistically significant difference in MCL-1 positive cells was observed from microinjection of any single agent or combination. Microinjection of the combination resulted in significantly less cells that were dual positive for MYC and MCL-1. Although both single agents also resulted in a decrease in MYC positive and MCL-1 positive cells, only the combination reached statistical significance.

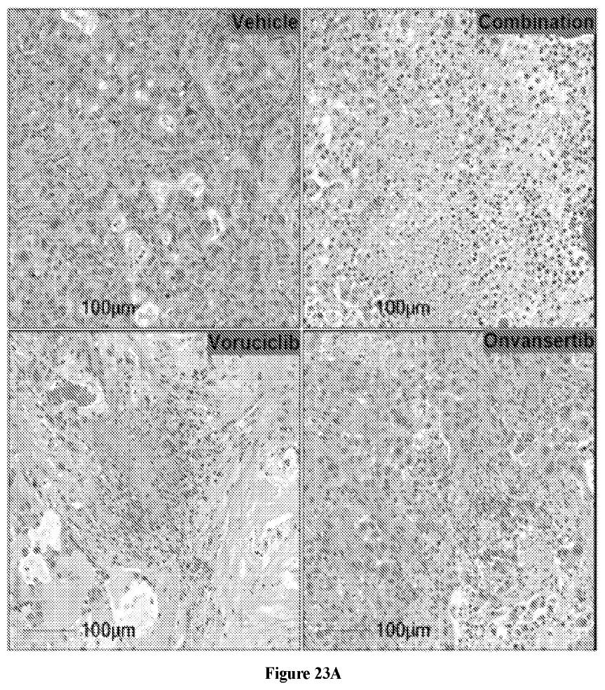

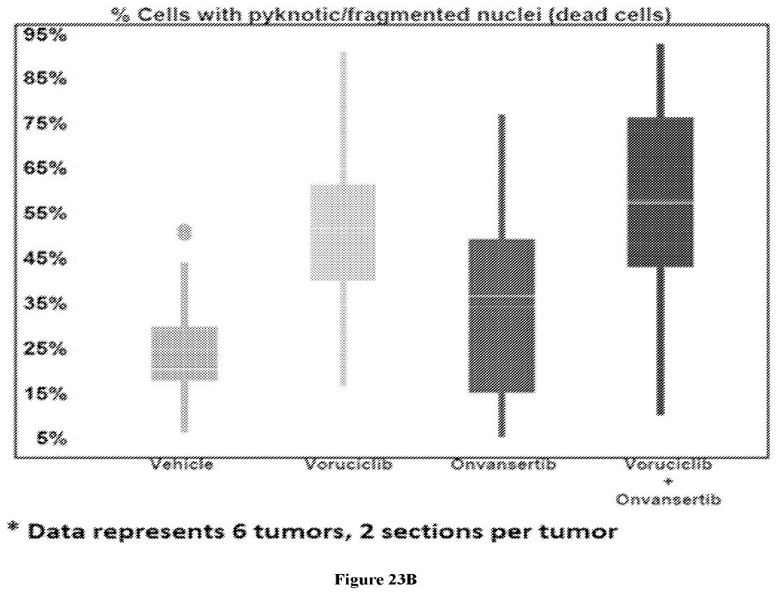

FIGS. 23A-23B show that the combination of voruciclib and onvansertib result in enhanced tumor cell death in HCC-44 (NSCLC) cells. Cell death around each microinjection site was measured by nuclear condensation, fragmentation, and clearance.

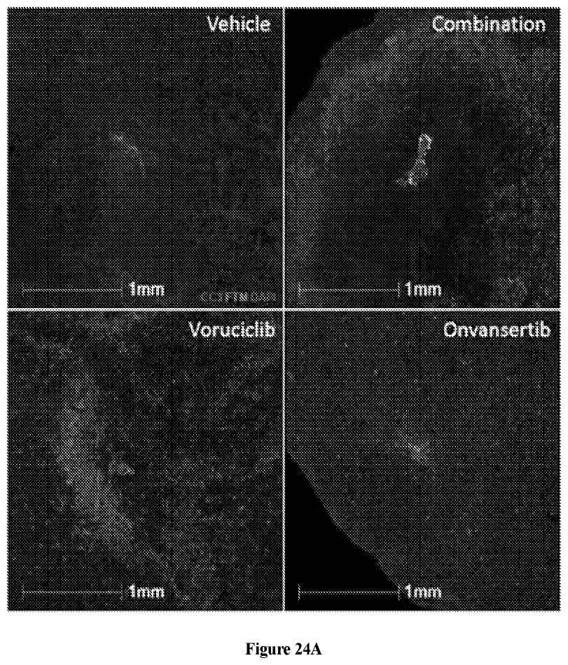

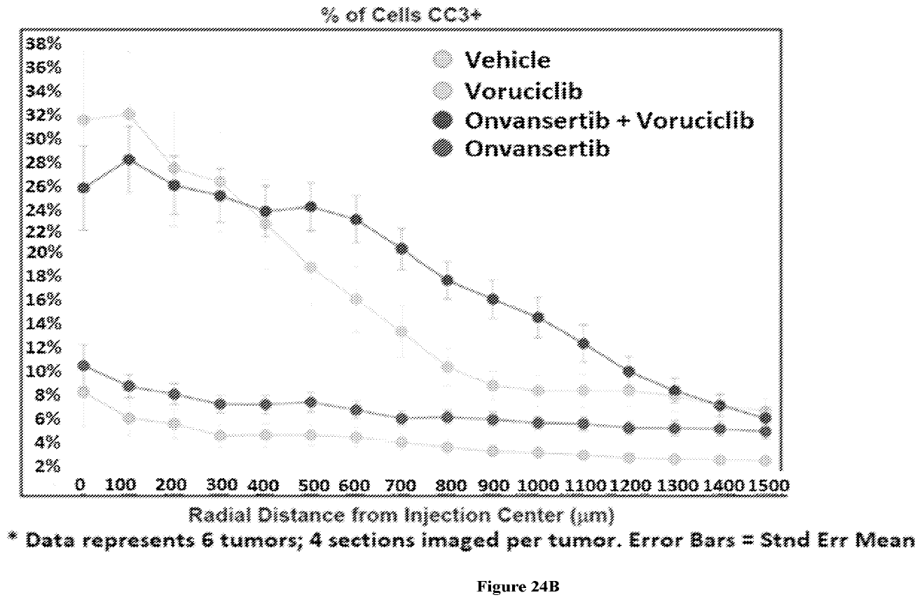

FIGS. 24A-24B show that the combination of voruciclib and onvansertib slightly increases apoptotic cells. The study was performed in HCC-44 (NSCLC) cells and apoptosis was measured by CC3+ cells.

### Example 8: 3D Spheroid Model for Combination Screening

Procedure:

Cells were seeded at day 0 with 2500 cells per well using a 96-well ultra low attachment plate. Immediately after seeding, plates were centrifuged for 10 min at 1000 RPM. The plates were incubated overnight at 37° C., 5% CO₂ and wells were visually inspected to confirm spheroid formation. Incubation was continued for 3 days. The cells were then exposed to the test compound(s) after 72 hours of incubation. 10 μl of the test compound was added at 10× the desired final concentration. The plates were then returned to the incubator at 37° C., 5% CO₂ until endpoint assay (day 6). Collection occurred after 72 (±4 hours) at day 6, pictures were taken, and plates were measured with the ATP-Lite assay.

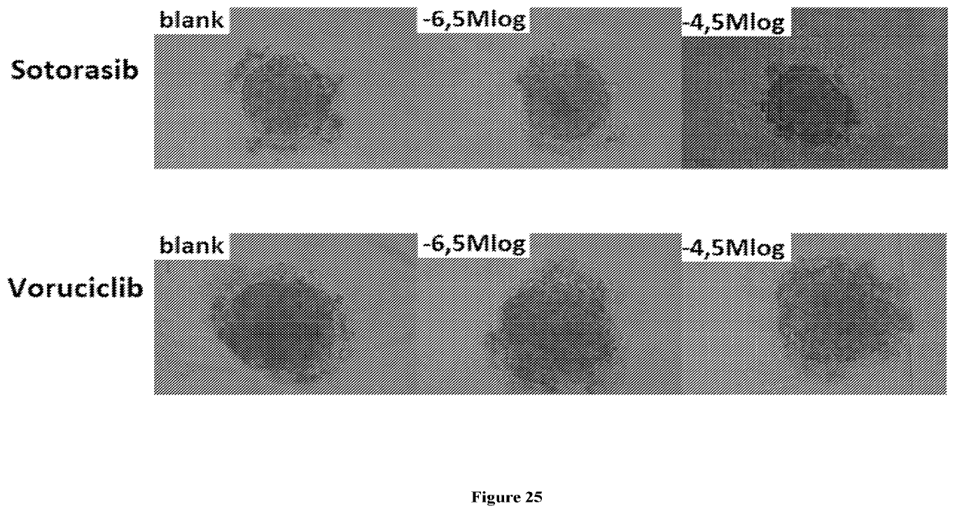

FIG. 25 depicts the 3D spheroids for sotorasib and voruciclib in NCI-H358 cells after 1 day of exposure.

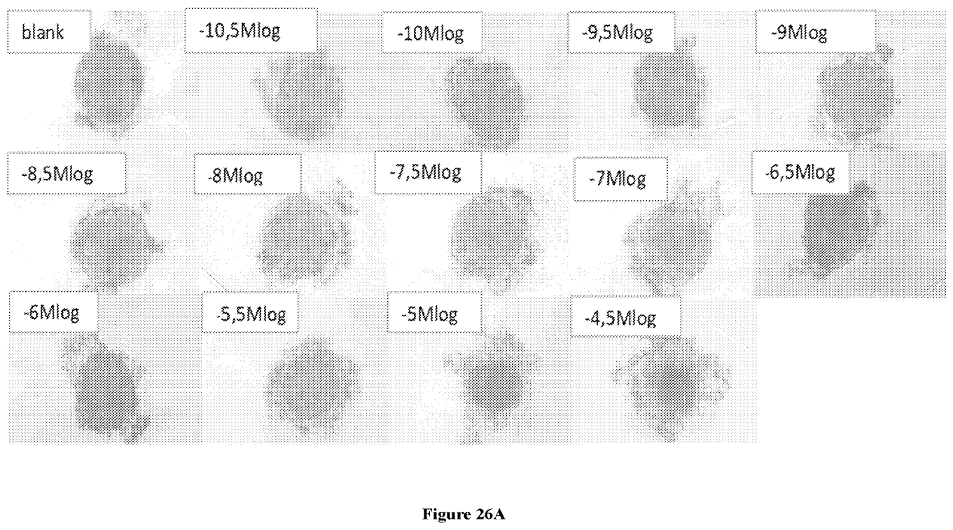

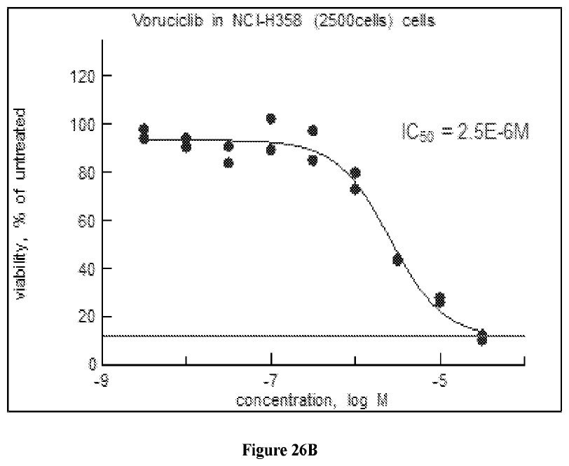

FIGS. 26A-26B depict the 3D spheroids for the voruciclib combination screening in NCI-H358 cells. The spheroids were grown in low attachment plates for 3 days before voruciclib treatment for 3 days. FIG. 26A provides pictures of the NCI-H358 cells after 3 days of exposure with voruciclib, before ATP-Lite measurement. FIG. 26B is a dose response curve of voruciclib in NCI-H358 cells, wherein the 2D culture was found to have an IC₅₀ of 0.6 μM.

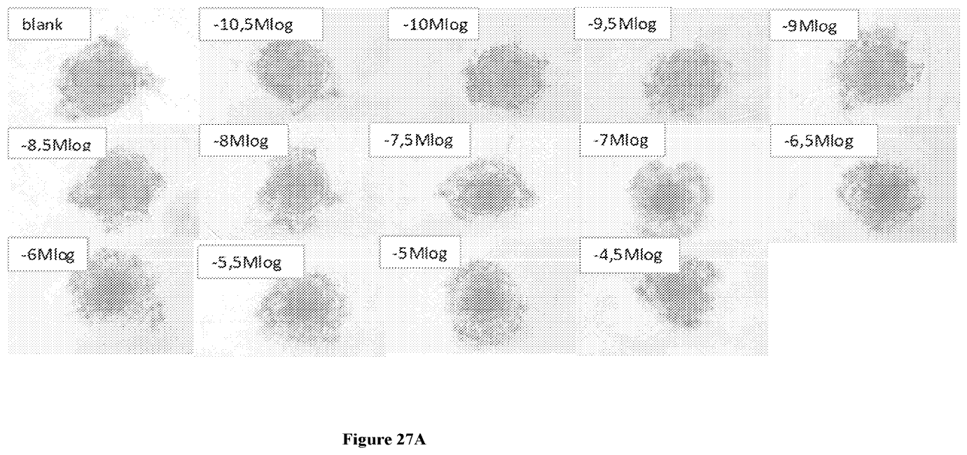

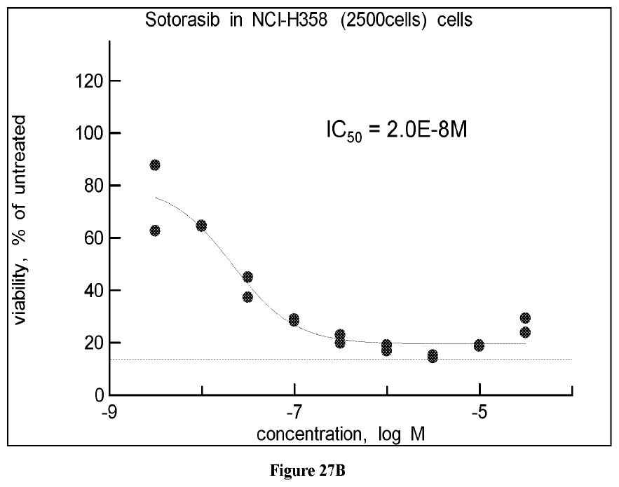

FIGS. 27A-27B depict the 3D spheroids for the sotorasib combination screening in NCI-H358 cells (NSCLC). FIG. 27A provides pictures of the NCI-H358 cells after 3 days of exposure with sotorasib, before ATP-Lite measurement. FIG. 27B is a dose response curve of sotorasib in NCI-H358 cells.

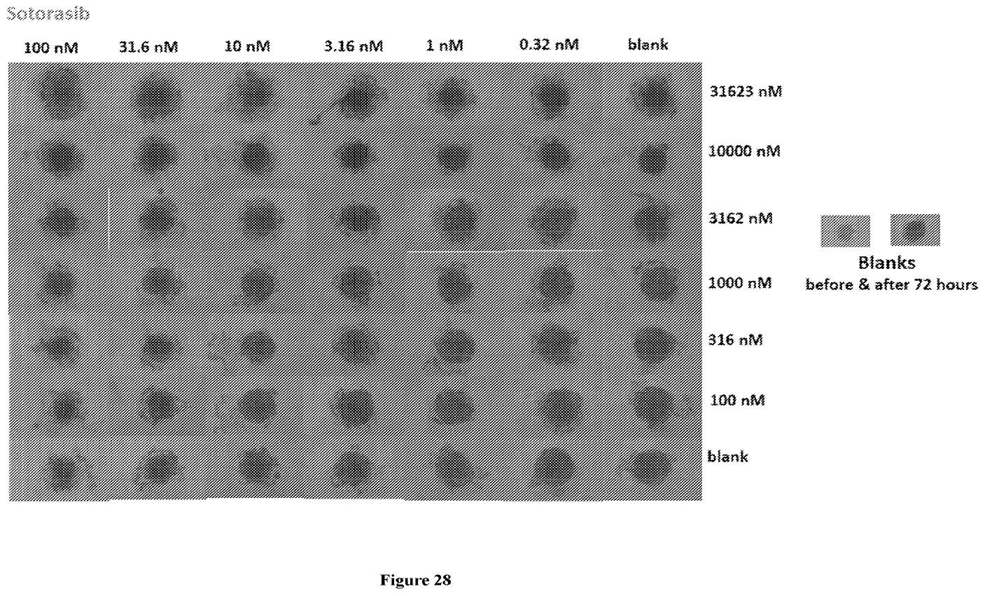

FIG. 28 depicts the 3D spheroids for the voruciclib+sotorasib combination screening in NCI-H358 cells (NSCLC). Pictures of the NCI-H358 cells were obtained after 3 days of exposure with the sotorasib and voruciclib combination.

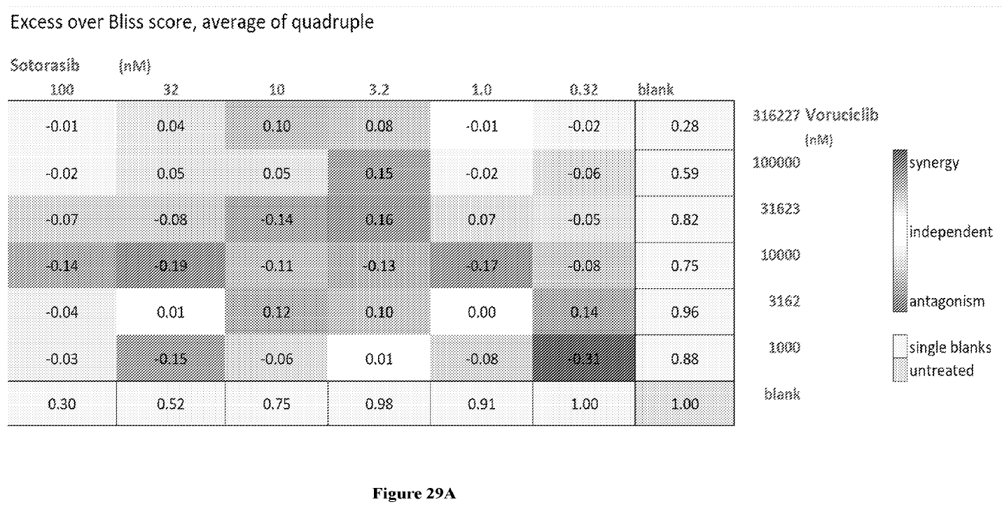

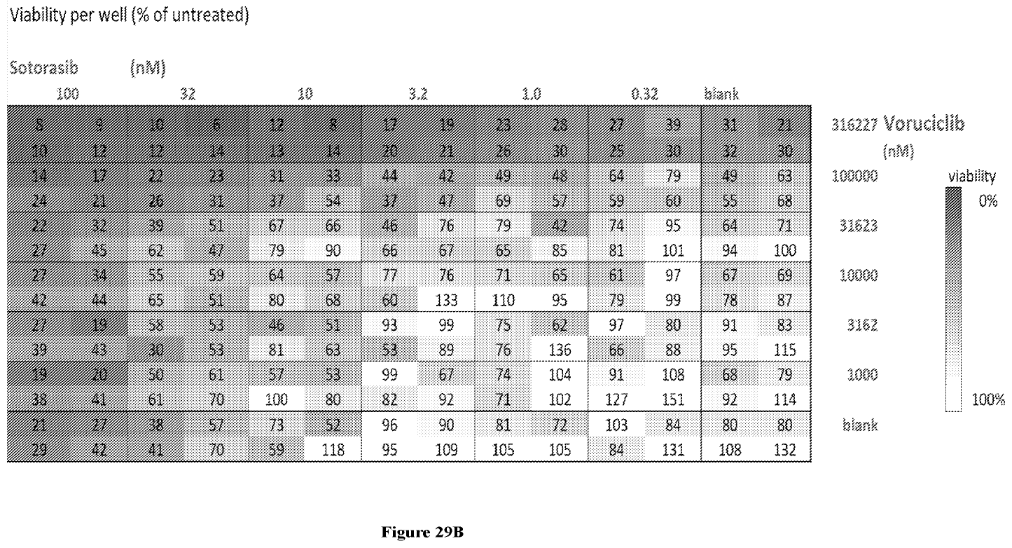

FIGS. 29A-29B depict the synergy with voruciclib+sotorasib (at higher voruciclib concentrations) in NCI-H358 3D spheroids. FIG. 29A is a chart of the excess over Bliss score.

FIG. 29B is a chart of the viability for well (% untreated).

### Example 9: Monotherapy Phase 1 Studies in Solid Tumors

2 weeks on/i week off schedule: 75 to 850 mg; 29 pts in dose escalation/expansion at 600 mg cohorts; 41% disease control rate; 1 PR and 8 SD lasting 2 to 6 months.

Daily continuously schedule: 75 to 500 mg; 39 pts in dose escalation/expansion at 350 mg cohorts; 31% disease control rate; 12 SD lasting a median of 15 weeks.

Safety profile: most common AEs involved GI tract; no evidence of myelosuppression.

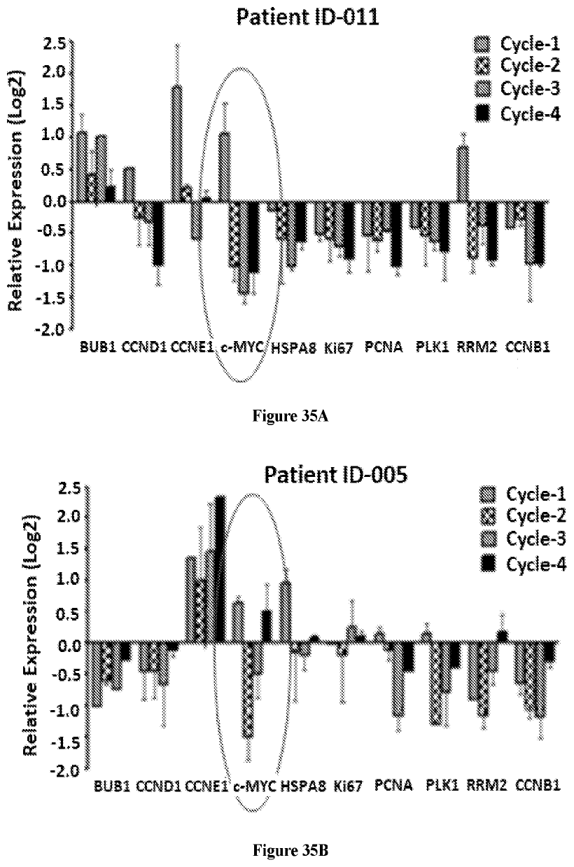

Decreased c-MYC Expression in Solid Tumors: 10 gene biomarkers evaluated in Phase 1 daily dosing study; c-MYC expression decreased in 17/25 patients (68%) tested (FIGS. 35A and 35B).

### Example 10: Study of Voruciclib+Vemurafenib in BRAF-Mut Advanced/Inoperable Malignant Melanoma

Voruciclib 150 mg daily plus vemurafenib 720 mg or 960 mg BID in 28-day cycles; 9 pts treated before study termination; 8 patients evaluable for efficacy; 5 patients were BRAFi refractory, response=PD; 3 patients were BRAF/MEK naïve, 1 CR and 2 PR ongoing for 3 to 14 months; most common AEs were fatigue, constipation, diarrhea, arthralgia and headache; 1 DLT=grade 3 fatigue.

### Example 11: CR in a Patient with Pulmonary Metastases

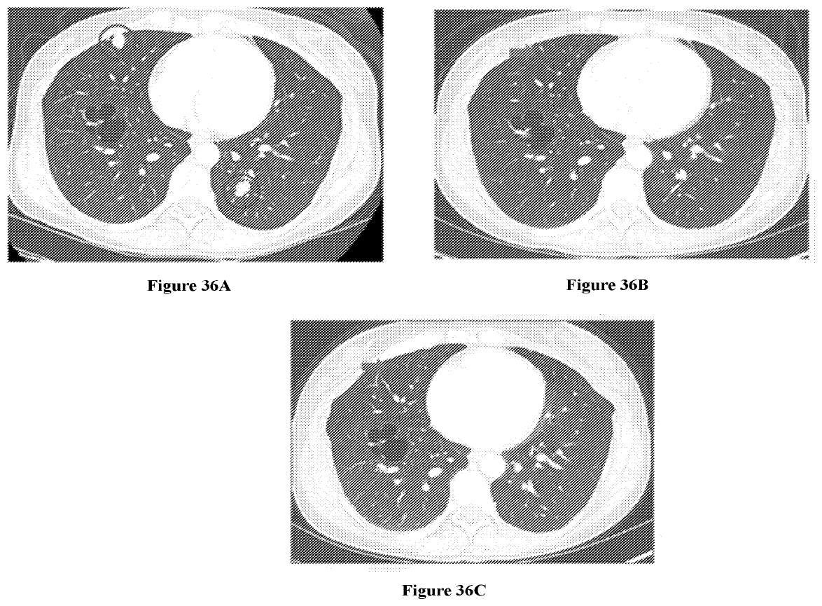

FIGS. 36A-36C illustrate CR in a Patient with Pulmonary Metastases; FIG. 36A: baseline CT scan; FIG. 36B: 2 months after starting the trial, radiological CR based on official radiological report; FIG. 36C: 14 months after starting the trail, patient remained on trial for 12 months only, and CR remained durable for 14 months.

### Example 12: Leveraging CDK9 Regulation of MCL1: Phase 1 Study in R/R B-Cell Malignancies and AML

Study population: Relapsed/Refractory B-cell malignancies; Relapsed/Refractory AML; Dose escalation with standard 3+3 design.

Endpoints: Safety and tolerability, Pharmacokinetics, Biologic correlative studies (BH3 profiling, MCL-1 expression, molecular mutations analysis), Response rates.

Voruciclib single agent dose escalation: 50 mg>100 mg>150 mg>200 mg.

Phase 1 Study in Hematologic Malignancies: 24 pts treated in 3 dose levels, 10 AML and 14 B-cell malignancies, No GI toxicity or neutropenia at doses studied, Favorable PK profile across all voruciclib studies (Half life 24-28 hours supports once-a-day dosing, Dose proportional Cmax and AUC; High volume of distribution indicates broad entry into tissues); Doses of 150-200 mg may achieve plasma concentrations sufficient to inhibit molecular target.

### Example 13: Voruciclib Shows Preferential Tumor Accumulation in Preclinical Model

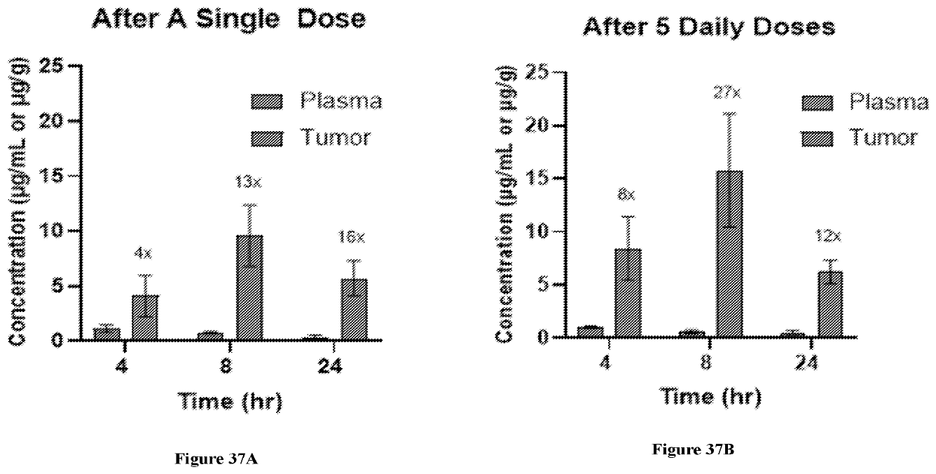

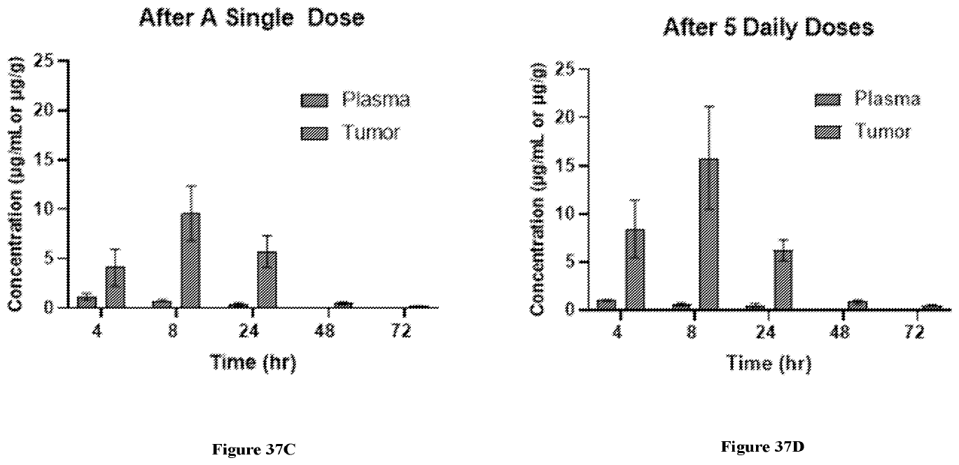

Voruciclib Shows Preferential Tumor Accumulation in Preclinical Model (FIGS. 37A-37D). HCT-116 CRC cell xenograft in SCID mice. 8 mice per time point (2 control, 6 orally dosed with voruciclib at 100 mpk). Animals were randomized into 2 groups when tumors reached 100 mm diameter. Group A assigned single dosing. Group B assigned 5 day dosing. 8 mice per time point (2 control, 6 orally dosed with voruciclib at 100 mpk). Drug concentration was measured in tumor and plasma after last dose at the following time points: 0, 4, 8, 24, 48, 72 hrs. The accumulation index of voruciclib in tumors after 5 days of repeat dosing was 1.45. Concentration of voruciclib in plasma is in units of μg/ml and in μg/g for tumors. Voruciclib fold increase in tumors relative to plasma are indicated.

Higher levels (>5 fold) of voruciclib were found in tumor at 8 hours compared to 24 hours post dosing. Negligible levels of voruciclib were observed in the plasma at 24 hours. Moreover, tumor to plasma ratio was found to be >5 fold at both time points.

### Example 14: Evidence of Biologic Activity in AML

Differentiation syndrome seen in 5 pts (50%) (Increased WBC without increased in blasts, bone pain, pulmonary symptoms; Response to corticosteroids).

Differentiation syndrome with ATRA, IDHi, and other AML targeted therapies.

### Example 15: Voruciclib Synergizes with Venetoclax in Venetoclax Sensitive and Resistant Cell Lines

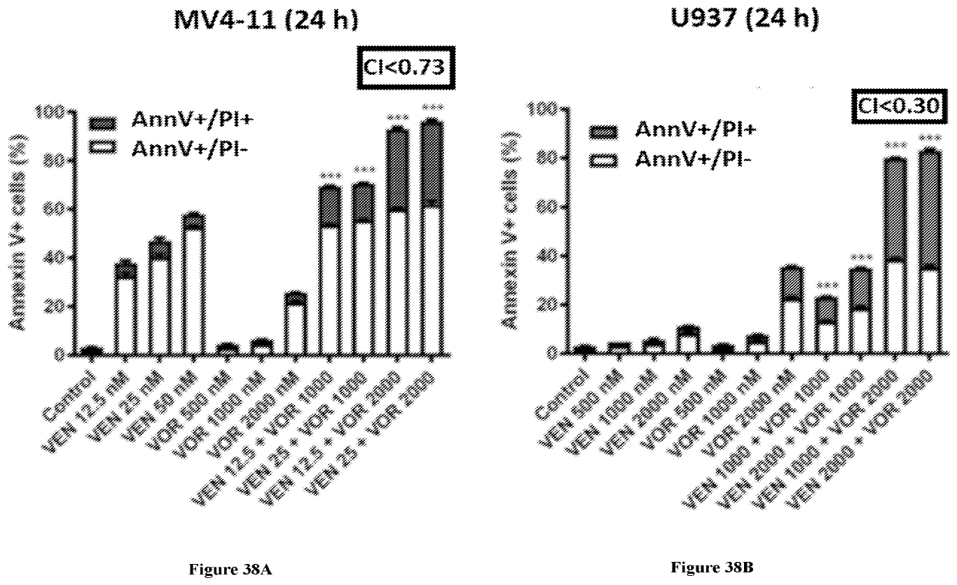

FIGS. 38A and 38B illustrate that Voruciclib Synergizes with Venetoclax in Venetoclax Sensitive and Resistant Cell Lines. FIG. 4A: Ven Sensitive; FIG. 4B: Ven Resistant.

Phase 1 Study of Voruciclib+Venetoclax in AML; Study population: Relapsed/Refractory B-cell malignancies; Relapsed/Refractory AML; Dose escalation with standard 3+3 design.
Endpoints: Safety and tolerability; Pharmacokinetics; Biologic correlative studies (BH3 profiling, MCL-1 expression, Molecular mutations analysis); Response rates.
Voruciclib single agent dose escalation: 50 mg>100 mg>150 mg>200 mg
Voruciclib+Venetoclax dose escalation: 100 mg>150 mg>200 mg.

### Example 16: Voruciclib Dosing Regimen

PK data for 2 weeks of daily dosing followed by 1 week without dosing in a 21-day cycle is presented Table 3, and PK data for daily dosing continuously is presented in Table 4.

TABLE 3
Descriptive Statistics (Mean ± Std. Dev) of PK Parameters of Voruciclib on Day 1 and Day 13

| | | Dose | | | | |
|---|---|---|---|---|---|---|
| | | 75 mg | 150 mg | 300 mg | 600 mg | 850 mg |
| Parameter | Day | (n = 3) | (n = 3) | (n = 3) | (n = 16) | (n = 4) |
| Tₘₐₓ(h)* | Day 1 | 4.000 | 4.000 | 6.000 | 4.000 | 9.000 |
| | | (2.000-4.000) | (2.000-12.000) | (4.000-12.000) | (2.000-24.000) | (4.000-12.000) |
| | Day 13 | 4.000 | 4.000 | 12.000 | 4.000 | 5.000 |
| | | (2.000-6.000) | (4.000-6.000) | (4.000-12.000) | (2.000-12.000) | (4.000-6.000)^{#} |
| Cₘₐₓ | Day 1 | 193.146 ± | 456.390 ± | 1003.518 ± | 1628.010 ± | 1732.214 ± |
| (ng/mL) | | 181.7593 | 160.4270 | 448.0968 | 1242.8176 | 330.5233 |
| | Day 13 | 313.116 ± | 895.925 ± | 2100.265 ± | 2944.112 ± | 4449.010 ± |
| | | 24.5507 | 247.2074 | 639.0394 | 1422.6062 | 111.7476^{#} |
| AUC₀₋ₜ | Day 1 | 2279.320 ± | 6980.121 ± | 15653.965 ± | 24823.697 ± | 31351.031 ± |
| (ng · h/mL) | | 1367.3669 | 2955.5550 | 1346.4779 | 18560.8532 | 5484.4228 |
| | Day 13 | 5286.217 ± | 12957.290 ± | 40419.748 ± | 43508.009 ± | 83939.127 ± |
| | | 423.6548 | 1236.0382 | 10734.1354 | 21192.6452 | 13229.6999^{#} |
| AUC_{0-∞} | Day 1 | 4209.822 ± | 15765.319 ± | 147671.801 ± | 61535.871 ± | 108619.189 ± |
| (ng · h/mL) | | 2206.6963 | 1432.9428^{#} | 167803.1221^{#} | 55710.1558^{€} | 40549.3234^{#} |
| | Day 13 | 8659.785 ± | 28167.439 ± | 59331.780 ± | 86004.296 ± | 170083.523 ± |
| | | 354.5380 | 13275.2871 | —^{\hat{ }} | 62687.4524^{§} | 93983.7693^{#} |
| AUC₀₋₂₄ | Day 1 | 2279.320 ± | 6980.121 ± | 15653.965 ± | 24823.697 ± | 31351.031 ± |
| (ng · h/mL) | | 1367.3669 | 2955.5550 | 1346.4779 | 18560.8532 | 5484.4228 |
| | Day 13 | 5286.217 ± | 12957.290 ± | 40419.748 ± | 43508.009 ± | 83939.127 ± |
| | | 423.6548 | 1236.0382 | 10734.1354 | 21192.6452 | 13229.6999^{#} |
| t_{1/2} (h) | Day 1 | 21.658 ± | 32.527 ± | 137.821 ± | 32.544 ± | 50.044 ± |
| | | 13.4942 | 21.8661^{#} | 173.8172^{#} | 37.9895^{€} | 10.1297^{#} |
| | Day 13 | 16.928 ± | 26.061 ± | 16.166 ± | 19.036 ± | 22.380 ± |
| | | 0.9072 | 19.9929 | —^{\hat{ }} | 14.8429^{§} | 14.2008^{#} |
| Vz/F | Day 1 | 1279.160 ± | 1653.836 ± | 3978.953 ± | 1552.707 ± | 2151.883 ± |
| (L) | | 833.7216 | 1757.3463^{#} | 5096.6355^{#} | 1962.2874^{€} | 162.8208^{#} |
| | Day 13 | 348.886 ± | 458.679 ± | 185.110 ± | 393.745 ± | 314.518 ± |
| | | 44.8521 | 393.3117 | —^{\hat{ }} | 255.5617^{§} | 157.8920^{#} |
| Cl/F | Day 1 | 42.217 ± | 25.301 ± | 19.255 ± | 36.935 ± | 27.881 ± |
| (L/h) | | 24.7244 | 13.8587^{#} | 1.5779 | 29.4853 | 5.8668 |
| | Day 13 | 14.247 ± | 11.649 ± | 7.762 ± | 24.177 ± | 10.254 ± |
| | | 1.1088 | 1.1334 | 1.9348 | 33.4293 | 1.6161^{#} |
| Accumulation | Day 13/ | 2.937 ± | 2.129 ± | 2.602 ± | 2.064 ± | 3.139 ± |
| Ratio | Day 1 | 1.5793 | 0.9716 | 0.7871 | 0.9112 | 1.2489^{#} |
| (AUC_{0-24 h}) | | | | | | |

*Median (Range) values were reported for Tₘₐₓ.
^{\hat{ }}n = 1,
^{#}n = 2,
^{€}n = 11 and
^{§}n = 15
Since there were inadequate time-points to characterize the elimination phase AUC_{0-∞}, t_{1/2} and Vz/F could not be computed for patients 106 (Day 1), 202 (Day 1), 204 (Day 1), 216 (Day 13), 302 (Day 1), 306 (Day 1), 409 (Day 13), 410 (Day 1), 418 (Day 1), 419 (Day 1), 501(Day 1) and 501(Day 13).

TABLE 4
Descriptive Statistics (Mean ± Std. Dev) of PK Parameters of Voruciclib on Day 1 and Day 15

| | | Dose | | | | |
|---|---|---|---|---|---|---|
| | | 75 mg | 150 mg | 250 mg | 350 mg | 500 mg |
| Parameter | Day | (n = 3) | (n = 3) | (n = 3) | (n = 24) | (n = 6) |
| Tmax(h)* | Day 1 | 4.000 | 6.000 | 4.000 | 6.000 | 6.000 |
| | | (4.000-6.000) | (4.000-12.000) | (2.000-6.000) | (1.000-12.000) | (2.000-24.000) |
| | Day 15 | 2.000 | 6.000 | 4.000 | 4.000 | 2.000 |
| | | (0.000-2.000) | (0.250-24.000) | (2.000-6.000) | (0.500-24.000)^{§} | (1.000-6.000)^{##} |
| Cmax (ng | Day 1 | 125.116 ± | 257.918 ± | 471.955 ± | 623.486 ± | 1135.913 ± |
| | | 76.1007 | 48.9739 | 176.8485 | 147.4429 | 502.3381 |
| | Day 15 | 331.174 ± | 754.927 ± | 1047.832 ± | 1493.904 ± | 2254.686 ± |
| | | 287.8394 | 119.5624 | 268.6660 | 537.5295^{§} | 1259.5916^{##} |
| AUC0-t | Day 1 | 1726.805 ± | 4852.919 ± | 6114.663 ± | 10395.509 ± | 16370.472 ± |
| (ng · h/mL) | | 929.4239 [symbol] | 1101.4055 | 2456.5442 | 3024.9074 | 6058.2118 |
| | Day 15 | 5567.102 ± | 16642.661 ± | 18537.142 ± | 26733.146 ± | 40465.337 ± |
| | | 5258.249 [symbol] | 2969.0833 | 6973.9509 | 7478.4641^{§} | 25259.1489^{##} |
| AUC0-∞ | Day 1 | 3424.383 ± | 15592.890 ± | 16383.699 ± | 133647.705 ± | 41032.540 ± |
| (ng · h/mL) | | 1737.493 [symbol] | 7140.1056^{#} | 7564.4777 | 342360.8544^{§} | 18507.8312^{†} |
| | Day 15 | 27200.362 ± | 164073.250 ± | 54524.054 ± | 164113.625 ± | 108301.704 ± |
| | | 14338.0479 | —^{\hat{ }} | 2108.8817^{#} | 236753.6624^{€} | 71929.6562^{†} |
| AUC0-24 | Day 1 | 1726.805 ± | 4852.919 ± | 6114.663 ± | 10388.095 ± | 16370.472 ± |
| (ng · h/mL) | | 929.4239 [symbol] | 1101.4055 | 2456.5442 | 3026.1945 | 6058.2118 |
| | Day 15 | 5567.102 ± | 16642.661 ± | 18537.142 ± | 26733.146 ± | 40465.337 ± |
| | | 5258.249 [symbol] | 2969.0833 | 6973.9509 | 7478.4641^{§} | 25259.1489^{##} |
| t½ (h) | Day 1 | 21.776 ± | 39.589 ± | 34.397 ± | 129.341 ± | 24.798 ± |
| | | 6.8124 | 9.6927^{#} | 21.5177 | 308.5582^{§} | 5.2455^{†} |
| | Day 15 | 358.828 ± | 131.733 ± | 38.750 ± | 72.548 ± | 42.366 ± |
| | | 559.9488 | —^{\hat{ }} | 18.5958^{#} | 86.4263^{€} | 25.7296^{†} |
| Vz/F (L) | Day 1 | 860.496 ± | 579.369 ± | 765.765 ± | 155.214 ± | 72.515 ± |
| | | 526.4529 | 130.7782^{#} | 461.4432 | 112.0200^{§} | 25.3428^{†} |
| | Day 15 | 981.869 ± | 173.749 ± | 258.903 ± | 77.571 ± | 51.024 ± |
| | | 1326.5232 [symbol] | —^{\hat{ }} | 133.0243^{#} | 80.6852^{€} | 24.6115^{†} |
| CL/F | Day 1 | 28.856 ± | 10.746 ± | 17.724 ± | 4.224 ± | 2.107 ± |
| (L/h) | | 20.5929 | 4.9209^{#} | 8.3061 | 3.7491^{§} | 0.85597^{†} |
| | Day 15 | 3.264 ± | 0.914 ± | 4.589 ± | 1.607 ± | 1.031 ± |
| | | 1.4856 | —^{\hat{ }} | 0.1775^{#} | 2.0264^{€} | 0.7311^{†} |
| Accumulati [symbol] | Day 15/ | 2.634 ± | 3.637 ± | 3.090 ± | 2.767 ± | 2.577 ± |
| (AUC0-t) | Day 1 | 1.6433 | 1.3888 | 0.4162 | 1.0640^{§} | 0.7557^{##} |

* Median (Range) values were reported for Tmax.
^{\hat{ }}n = 1,
^{#}n = 2,
^{†}n = 4,
^{##}n = 5,
^{€}n = 13 and
^{§}n = 18
Since there were inadequate time-points to characterize the elimination phase AUC0-□, t_{1/2} and Vz/F could not be computed for patients 004 (Day 1), 004 (Day 15), 009 (Day 15), 011 (Day 1), 011 (Day 15), 023 (Day 1), 024 (Day 1), 026 (Day 1), 031 (Day 1), 005 (Day 15), 031 (Day 15), 032 (Day 15), 035 (Day 1), 035 (Day 15), 036 (Day 15), 013(Day 1), [symbol] 13 [symbol] ay 15) and 014 (Day 1). [symbol]
indicates data missing or illegible when filed

Voruciclib half life is designated as t½ in the tables (expressed in hours). Steady state (i.e., Day 13-15) half-life ranges from 16 hours to 358 hours, with an average of 24 to 48 hours. There is interpatient variability, explaining the outlier values. When the drug is stopped, it takes ˜5 half-lives, or 5-10 days for the drug to be eliminated from the plasma.

Voruciclib volume of distribution is designated as Vz/F (expressed in liters). Steady state (i.e., Day 13-15) volume of distribution ranges from 185 L to 982 L, with an average of ˜300 L (and outlier values). Blood volume is ˜5 L, and thus, in some embodiments, voruciclib volume of distribution is ˜60 times larger than the blood volume, indicating broad distribution into tissues. When the drug is stopped it takes an additional 3 days to clear from the tissue after it clears the plasma.

Without wishing to be bound by any particular theory, it is believed that by using a 14 days on therapy followed by 14 days off therapy, there is enough time for the drug to be eliminated from the plasma (Day 19 to Day 24) and another 3 days (Day 22 to Day 27) to be eliminated for the tissues, thereby preventing accumulation into tissue with continuous daily dosing, and potential toxicities. Without wishing to be bound by any particular theory, it is believed that voruciclib dosing on a 14 days on/14 days off schedule can prevent tissue toxicity. In some embodiments, such dosing regimen can match the dosing schedule of a combination drug.

While certain embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

### REFERENCES

Lai et al., Sensitivity of oncogenic KRAS expressing cells to CDK9 inhibition identified by a phenotypic compound screen, AACR, Session PO.ET01.02—Novel Therapeutic Approaches, 6425;
Luedtke et al., Inhibition of CDK9 by voruciclib synergistically enhances cell death induced by the Bcl-2 selective inhibitor venetoclax in preclinical models of acute myeloid leukemia, Signal Transduction and Targeted Therapy (2020) 5:17;
Blake et al., Application of a MYC degradation screen identifies sensitivity to CDK9 inhibitors in KRAS-mutant pancreatic cancer, Sci. Signal. 12, eaav7259 (2019); Hobbs G A, Der C J, and Rossman, K L. (2016) J Cell Sci. 129(7): 1287-1292 Hobbs G A, Der C J. RAS Mutations Are Not Created Equal. Cancer Discov. 2019 June; 9(6):696-698
Blake D R, et al. Application of a MYC degradation screen identifies sensitivity to CDK9 inhibitors in KRAS-mutant pancreatic cancer. Sci Signal. 2019 Jul. 16; 12(590):eaav7259 Kalkat M, et al. MYC Deregulation in Primary Human Cancers. Genes (Basel). 2017 May 25; 8(6):151
Dey J, et al. Voruciclib, a clinical stage oral CDK9 inhibitor, represses MCL-1 and sensitizes high-risk Diffuse Large B-cell Lymphoma to BCL2 inhibition. Sci Rep. 2017 Dec. 21; 7(1):18007
Luedtke D A, et al. Inhibition of CDK9 by voruciclib synergistically enhances cell death induced by the Bcl-2 selective inhibitor venetoclax in preclinical models of acute myeloid leukemia. Signal Transduct Target Ther. 2020 Feb. 26; 5(1):17.

1. A method of treating a KRAS mutant cancer comprising administering to a subject in need thereof a therapeutically effective amount of a compound of Formula Ib: 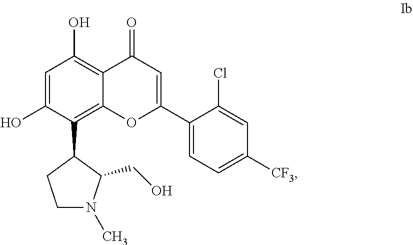 [SMILES: CN1CC[C@@H](c2c(O)cc(O)c3c(=O)cc(-c4ccc(C(F)(F)F)cc4Cl)oc23)[C@@H]1CO]
or a pharmaceutically acceptable salt thereof.

2. The method of claim 1, further comprising administering to the subject an additional anticancer agent.

3. (canceled)

4. The method of claim 2, wherein the anticancer agent is selected from Sotorasib (AMG510), Adagrasib (MRTX849), Onvansertib, Volasertib, and ME-344.

5. The method of claim 2, wherein the anticancer agent is selected from a KRAS inhibitor, a TKI+RAF inhibitor, a RAF inhibitor, a RAF+MEK inhibitor, a MEK inhibitor, and an ERK inhibitor.

6. The method of claim 1, wherein the KRAS mutant cancer is characterized by a mutation selected from G12A, G12C, G12D, G12S, G12V, G13C, G13D, and Q61H.

7. The method of claim 1, wherein the cancer is selected from acute myeloid leukemia (AML), chronic myeloid leukemia (CML), acute lymphocytic lymphoma (ALL), and chronic lymphocytic leukemia (CLL), diffuse large B-cell lymphoma (DLBCL), primary mediastinal B-cell lymphoma, intravascular large B-cell lymphoma, follicular lymphoma, small lymphocytic lymphoma (SLL), mantle cell lymphoma, marginal zone B-cell lymphomas, extranodal marginal zone B-cell lymphomas, nodal marginal zone B-cell lymphoma, splenic marginal zone B-cell lymphoma, Burkitt lymphoma, lymphoplasmacytic lymphoma, and primary central nervous system lymphoma.

8. The method of claim 1, wherein the cancer is selected from pancreatic cancer, lung cancer, colorectal cancer, esophageal cancer, ovarian cancer, NSCLC, SCLC, CRC, TNBC, melanoma, breast cancer, and liver cancer.

9. (canceled)

10. The method of claim 1, wherein the salt of the compound of Formula Ib is (+)-trans-2-(2-chloro-4-trifluoromethylphenyl)-5,7-dihydroxy-8-(2-hydroxymethyl-1-methyl-pyrrolidin-3-yl)-chromen-4-one hydrochloride.

11. The method of claim 1, wherein the compound of Formula Ib is administered in a crystal form.

12. The method of claim 11, wherein the crystal form comprises a malonate of the compound of Formula Ib, a hydrated malonate of the compound of Formula Ib, or an anhydrous malonate of the compound of Formula Ib, and is characterized by an X-ray powder diffraction pattern including one or more peaks selected from 7.30°±0.2°, 13.58°±0.2°, 14.06°±0.2°, 15.18°±0.2°, 15.66°±0.2°, 17.50°±0.2°, 18.94°±0.2°, 19.54°±0.2°, 22.22°±0.2°, 23.38°±0.2°, 24.10°±0.2°, 24.98°±0.2°, 25.94°±0.2°, 27.26°±0.2°, 28.50°±0.2°, and 32.82°±0.2° 2θ.

13. (canceled)

14. The method of claim 11, wherein the crystal form comprises a dibenzoyl-tartrate of the compound of Formula Ib, an anhydrous dibenzoyl-tartrate of the compound of Formula Ib, or a hydrated dibenzoyl-tartrate of the compound of Formula Ib, and is characterized by an X-ray powder diffraction pattern including one or more peaks selected from 5.06°±0.2°, 6.42°±0.2°, 9.34°±0.2°, 10.14°±0.2°, 12.30°±0.2°, 13.66°±0.2°, 14.14°±0.2°, 15.82°±0.2°, 17.02°±0.2°, 19.74°±0.2°, 20.38°±0.2°, 21.82°±0.2°, 22.66°±0.2°, 24.620°±0.20, 25.780°±0.20, 26.580°±0.20, 28.660°±0.20, and 29.98°±0.2° 2θ.

15. (canceled)

16. The method of claim 11, wherein the crystal form comprises a phosphate of the compound of Formula Ib, a hydrated phosphate of the compound of Formula Ib, or an anhydrous phosphate of the compound of Formula Ib, and is characterized by an X-ray powder diffraction pattern including one or more peaks selected from 4.94°±0.2°, 6.78°±0.2°, 9.34°±0.2°, 10.94°±0.2°, 12.70°±0.2°, 13.38°±0.2°, 14.90°±0.2°, 15.66°±0.2°, 17.54°±0.2°, 18.82°±0.2°, 22.02°±0.2°, 23.98°±0.2°, 24.78°±0.2°, 25.30°±0.2°, 26.66°±0.2°, and 29.98°±0.2° 2θ.

17. (canceled)

18. The method of claim 11, wherein the crystal form comprises an oxalate of the compound of Formula Ib, a hydrated oxalate of the compound of Formula Ib, or an anhydrous oxalate of the compound of Formula Ib, and is characterized by an X-ray powder diffraction pattern including one or more peaks selected from 6.86°±0.2°, 12.66°±0.2°, 13.58°±0.2°, 14.74°±0.2°, 15.98°±0.2°, 19.38°±0.2°, 23.94°±0.2°, 24.78°±0.2°, and 25.94°±0.2° 2θ.

19. (canceled)

20. The method of claim 11, wherein the crystal form comprises a napadisylate of the compound of Formula Ib, a hydrated napadisylate of the compound of Formula Ib, or an anhydrous napadisylate of the compound of Formula Ib, and is characterized by an X-ray powder diffraction pattern including one or more peaks selected from 9.02°±0.2°, 10.50°±0.2°, 11.06°±0.2°, 12.30°±0.2°, 12.82°±0.2°, 13.90°±0.2°, 14.82°±0.2°, 15.30°±0.2°, 15.94°±0.2°, 17.26°±0.2°, 19.34°±0.2°, 20.62°±0.2°, 22.18°±0.2°, 22.86°±0.2°, 24.58°±0.20, 25.420°±0.20, 25.860°±0.20, 27.380°±0.20, and 28.66°±0.2° 2θ.

21. (canceled)

22. The method of claim 1, wherein the compound of Formula Ib is administered at a free base dosage of:
about 100 mg daily, about 150 mg daily, about 200 mg daily, about 250 mg daily, about 300 mg daily, or about 350 mg daily; or
about 100 mg every other day, about 150 mg every other day, about 200 mg every other day, about 250 mg every other day, about 300 mg every other day, about 350 mg every other day, about 400 mg every other day, about 450 mg every other day, or about 500 mg every other day.

23. (canceled)

24. The method of claim 1, wherein the compound of Formula Ib is administered:
daily for about one day, about two days, about three days, about 4 days, about 5 days, about 6 days, about 7 days, about 8 days, about 9 days, about 10 days, about 11 days, about 12 days, about 13 days, or about 14 days; or
every other day for about one day, about two days, about three days, about 4 days, about 5 days, about 6 days, about 7 days, about 8 days, about 9 days, about 10 days, about 11 days, about 12 days, about 13 days, or about 14 days.

25. (canceled)

26. The method of claim 1, wherein the compound of Formula Ib is administered:
daily for about one week, about two weeks, about three weeks, or about 4 weeks; or
every other day for about one week, about two weeks, about three weeks, or about 4 weeks.

27. (canceled)

28. The method of claim 1, wherein the compound of Formula Ib administration is paused for about one day, about two days, about three days, about 4 days, about 5 days, about 6 days, about 7 days, about 8 days, about 9 days, about 10 days, about 11 days, about 12 days, about 13 days, about 14 days, about three weeks, or about four weeks.

29. (canceled)

30. The method of claim 1, wherein the compound of Formula Ib is administered on a 14 days on/14 days off schedule.

31. The method of claim 1, wherein the compound of Formula Ib is administered for about one month, about two months, about three months, about 4 months, about 5 months, about 6 months, about 7 months, about 8 months, about 9 months, about 10 months, about 11 months, or about 12 months.
